SEQUENCE LISTING (28 sequences)

SEQ ID NO: 1 - protein, 767 aa, Oryza sativa
MPVFTASFQCVTLFGQPASAADAQPLLQGQRPFLHLHARRRRPCGPMLISKSPPYPASEETREWEAEGQHEHTDELRETTTTMIDGIRTALRSIGEGEISISAYDTSLVALLKRLDGGDGPQFPSTIDWIVQNQLPDGSWGDASFFMMGDRIMSTLACVVALKSWNIHTDKCERGLLFIQENMWRLAHEEEDWMLVGFEIALPSLLDMAKDLDLDIPYDEPALKAIYAERERKLAKIPRDVLHAMPTTLLHSLEGMVDLDWEKLLKLRCLDGSFHCSPASTATAFQQTGDQKCFEYLDGIVKKFNGGVPCIYPLDVYERLWAVDRLTRLGISRHFTSEIEDCLDYIFRNWTPDGLAHTKNCPVKDIDDTAMGFRLLRLYGYQVDPCVLKKFEKDGKFFCLHGESNPSSVTPMYNTYRASQLKFPGDDGVLGRAEVFCRSFLQDRRGSNRMKDKWAIAKDIPGEVEYAMDYPWKASLPRIETRLYLDQYGGSGDVWIGKVLHRMTLFCNDLYLKAAKADFSNFQKECRVELNGLRRWYLRSNLERFGGTDPQTTLMTSYFLASANIFEPNRAAERLGWARVALLADAVSSHFRRIGGPKNLTSNLEELISLVPFDDAYSGSLREAWKQWLMAWTAKESSQESIEGDTAILLVRAIEIFGGRHVLTGQRPDLWEYSQLEQLTSSICRKLYRRVLAQENGKSTEKVEEIDQQLDLEMQELTRRVLQGCSAINRLTRETFLHVVKSFCYVAYCSPETIDNHIDKVIFQDVI

SEQ ID NO: 2 - protein, 807 aa, Euphobia peplus
MAAAANPSNSILNHHLLSSAAARSVSTSQLLFHSRPLVLSGAKDKRDSFVFRIKCSAVSNPRIQEQTDVFQKNGLPVIKWHEFVETDIDHEQVSKVSVSNEIKKRVESIKAILESMEDGDITISAYDTAWVALVEDINGSGAPQFPASLQWIANNQLPDGSWGDAEIFTAHDRILNTLSCVVALKSWNIHPDMCERGMKYFRENLCKLEDENIEHMPIGFEVAFPSLLELAKKLEIQVPEDSPVLKDVYDSRNLKLKKIPKDIMHKVPTTLLHSLEGMPGLEWEKLLKLQSKDGSFLFSPSSTAYALMQTKDQNCLEYLTKIVHKFNGGVPNVYPVDLFEHIWAVDRLQRLGISRYFQPQLKDSVDYVARYWEEDGICWARNSSVHDVDDTAMGFRVLRSFGHHVSADVFKHFKKGDTFFCFAGQSTQAVTGMYNLLRASQLMFPGEKILEEAKQFSSAFLKVKQDANEVLDKWIITKDLPGEVKYALDIPWYASLPRVESRFYIEQYGGSDDVWIGKTLYRMPIVNNDEYLKLAKLDYNNCQAVHRSEWDNIQKWYEESDLAEFGVSRREILMAYYLAAASIFEPEKSRERIAWAKTSVLLNTIQAYFHENNSTIHEKAAFVQLFKSGFAINARKLEGKTMEKLGRIIVGTLNDVSLDTAMAYGKDISRDLRHAWDICLQKWEESGDMHQGEAQLIVNTINLTSDAWNFNDLSSHYHQFFQLVNEICYKLRKYKKNKVNDKKKTTTPEIESHMQELVKLVLESSDDLDSNLKQIFLTVARSFYYPAVCDAGTINYHIARVLFERVY

SEQ ID NO: 3 - protein, 827 aa, Zea Maiz
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 4 - protein, 807 aa, Tripterygium Wilfordii
MHSLLMKKVIMYSSQTTHVFPSPLHCTIPKSSSFFLDAPVVRLHCLSGHGAKKKRLHFDIQQGRNAISKTHTPEDLYAKQEYSVPEIVKDDDKEEEVVKIKEHVDIIKSMLSSMEDGEISISAYDTAWVALIQDIHNNGAPQFPSSLLWIAENQLPDGSWGDSRVFLAFDRIINTLACVVALKSWNVHPDKCERGISFLKENISMLEKDDSEHMLVGFEFGFPVLLDMARRLGIDVPDDSPFLQEIYVQRDLKLKRIPKDILHNAPTTLLHSLEAIPDLDWTKLLKLQCQDGSLLFSPSSTAMAFINTKDENCLRYLNYVVQRFNGGAPTVYPYDLFEHNWAVDRLQRLGISRFFQPEIRECMSYVYRYWTKDGIFCTRNSRVHDVDDTAMGFRLLRLHGYEVHPDAFRQFKKGCEFICYEGQSHPTVTVMYNLYRASQLMFPEEKILDEAKQFTEKFLGEKRSANKLLDKWIITKDLPGEVGFALDVPWYASLPRVEARFFIQHYGGEDDVWLDKALYRMPYVNNNVYLELAKLDYNYCQALHRTEWGHIQKWYEECKPRDFGISRECLLRAYFMAAASIFEPERSMERLAWAKTAILLEIIVSYFNEVGNSTEQRIAFTTEFSIRASPMGGYINGRKLDKIGTTQELIQMLLATIDQFSQDAFAAYGHDITRHLHNSWKMWLLKWQEEGDRWLGEAELLIQTINLMADHKIAEKLFMGHTNYEQLFSLTNKVCYSLGHHELQNNKELEHDMQRLVQLVLTNSSDGIDSDIKKTFLAVAKRFYYTAFVDPETVNVHIAKVLFERVD

SEQ ID NO: 5 - protein, 786 aa, Coleus forskohlii
MGSLSTMNLNHSPMSYSGILPSSSAKAKLLLPGCFSISAWMNNGKNLNCQLTHKKISKVAEIRVATVNAPPVHDQDDSTENQCHDAVNNIEDPIEYIRTLLRTTGDGRISVSPYDTAWVALIKDLQGRDAPEFPSSLEWIIQNQLADGSWGDAKFFCVYDRLVNTIACVVALRSWDVHAEKVERGVRYINENVEKLRDGNEEHMTCGFEVVFPALLQRAKSLGIQDLPYDAPVIQEIYHSREQKSKRIPLEMMHKVPTSLLFSLEGLENLEWDKLLKLQSADGSFLTSPSSTAFAFMQTRDPKCYQFIKNTIQTFNGGAPHTYPVDVFGRLWAIDRLQRLGISRFFESEIADCIAHIHRFWTEKGVFSGRESEFCDIDDTSMGVRLMRMHGYDVDPNVLKNFKKDDKFSCYGGQMIESPSPIYNLYRASQLRFPGEQILEDANKFAYDFLQEKLAHNQILDKWVISKHLPDEIKLGLEMPWYATLPRVEARYYIQYYAGSGDVWIGKTLYRMPEISNDTYHELAKTDFKRCQAQHQFEWIYMQEWYESCNMEEFGISRKELLVAYFLATASIFELERANERIAWAKSQIISTIIASFFNNQNTSPEDKLAFLTDFKNGNSTNMALVTLTQFLEGFDRYTSHQLKNAWSVWLRKLQQGEGNGGADAELLVNTLNICAGHIAFREEILAHNDYKTLSNLTSKICRQLSQIQNEKELETEGQKTSIKNKELEEDMQRLVKLVLEKSRVGINRDMKKTFLAVVKTYYYKAYHSAQAIDNHMFKVLFEPVA

SEQ ID NO: 6 - protein, 785 aa, Salvia scarea
MTSVNLSRAPAAITRRRLQLQPEFHAECSWLKSSSKHAPLTLSCQIRPKQLSQIAELRVTSLDASQASEKDISLVQTPHKVEVNEKIEESIEYVQNLLMTSGDGRISVSPYDTAVIALIKDLKGRDAPQFPSCLEWIAHHQLADGSWGDEFFCIYDRILNTLACVVALKSWNLHSDIIEKGVTYIKENVHKLKGANVEHRTAGFELVVPTFMQMATDLGIQDLPYDHPLIKEIADTKQQRLKEIPKDLVYQMPTNLLYSLEGLGDLEWERLLKLQSGNGSFLTSPSSTAAVLMHTKDEKCLKYIENALKNCDGGAPHTYPVDIFSRLWAIDRLQRLGISRFFQHEIKYFLDHIESVWEETGVFSGRYTKFSDIDDTSMGVRLLKMHGYDVDPNVLKHFKQQDGKFSCYIGQSVESASPMYNLYRAAQLRFPGEEVLEEATKFAFNFLQEMLVKDRLQERWVISDHLFDEIKLGLKMPWYATLPRVEAAYYLDHYAGSGDVWIGKSFYRMPEISNDTYKELAILDFNRCQTQHQLEWIHMQEWYDRCSLSEFGISKRELLRSYFLAAATIFEPERTQERLLWAKTRILSKMITSFVNISGTTLSLDYNFNGLDEIISSANEDQGLAGTLLATFHQLLDGFDIYTLHQLKHVWSQWFMKVQQGEGSGGEDAVLLANTLNICAGLNEDVLSNNEYTALSTLTNKICNRLAQIQDNKILQVVDGSIKDKELEQDMQALVKLVLQENGGAVDRNIRHTFLSVSKTFYYDAYHDDETTDLHIFKVLFRPVV

SEQ ID NO: 7 - protein, 815 aa, Tripterygium Wilfordii
MFMSSSSSSHARRPQLSSFSYLHPPLPFPGLSFFNTRDKRVNFDSTRIICIAKSKPARTTPEYSDVLQTGLPLIVEDDIQEQEEPLEVSLENQIRQGVDIVKSMLGSMEDGETSISAYDTAWVALVENIHHPGSPQFPSSLQWIANNQLPDGSWGDPDVFLAHDRLINTLACVIALKKWNIHPHKCKRGLSFVKENISKLEKENEEHMLIGFEIAFPSLLEMAKKLGIEIPDDSPALQDIYTKRDLKLTRIPKDKMHNVPTTLLHSLEGLPDLDWEKLVKLQFQNGSFLFSPSSTAFAFMHTKDGNCLSYLNDLVHKFNGGVPTAYPVDLFEHIWSVDRLQRLGISRFFHPEIKECLGYVHRYWTKDGICWARNSRVQDIDDTAMGFRLLRLHGYEVSPDVFKQFRKGDEFVCFMGQSNQAITGIYNLYRASQMMFPEETILEEAKKFSVNFLREKRAASELLDKWIITKDLPNEVGFALDVPWYACLPRVETRLYIEQYGGQDDVWIGKTLYRMPYVNNNVYLELAKLDYNNCQSLHRIEWDNIQKWYEGYNLGGFGVNKRSLLRTYFLATSNIFEPERSVERLTWAKTAILVQAIASYFENSREERIEFANEFQKFPNTRGYINGRRLDVKQATKGLIEMVFATLNQFSLDALVVHGEDITHHLYQSWEKWVLTWQEGGDRREGEAELLVQTINLMAGHTHSQEEELYERLFKLTNTVCHQLGHYHHLNKDKQPQQVEDNGGYNNSNPESISKLQIESDMRELVQLVLNSSDGMDSNIKQTFLAVTKSFYYTAFTHPGTVNYHIAKVLFERVV

SEQ ID NO: 8 - protein, 815 aa, Tripterygium Wilfordii
MFMSSSSSSHARRPQLSSFSYLHPPLPFPGLSFFNTRDKRVNFDSTRIICIAKSKPARTTPEYSDVLQTGLPLIVEDDIQEQEEPLEVSLENQIRQGVDIVKSMLGSMEDGETSISAYDTAWVALVENIHHPGSPQFPSSLQWIANNQLPDGSWGDPDVFLAHDRLINTLACVIALKKWNIHPHKCKRGLSFVKENISKLEKENEEHMLIGFEIAFPSLLEMAKKLGIEIPDDSPALQDIYTKRDLKLTRIPKDIMHNVPTTLLYSLEGLPSLDWEKLVKLQCTDGSFLFSPSSTACALMHTKDGNCFSYINNLVHKFNGGVPTVYPVDLFEHIWCVDRLQRLGISRFFHPEIKECLGYVHRYWTKDGICWARNSRVQDIDDTAMGFRLLRLHGYEVSPDVFKQFRKGDEFVCFMGQSNQAITGIYNLYRASQMMFPEETILEEAKKFSVNFLREKRAASELLDKWIITKDLPNEVGFALDVPWYACLPRVETRLYIEQYGGQDDVWIGKTLYRMPYVNNNVYLELAKLDYNNCQSLHRIEWDNIQKWYEGYNLGGFGVNKRSLLRTYFLATSNIFEPERSVERLTWAKTAILVQAIASYFENSREERIEFANEFQKFPNTRGYINGRRLDVKQATKGLIEMVFATLNQFSLDALVVHGEDITHHLYQSWEKWVLTWQEGGDRREGEAELLVQTINLMAGHTHSQEEELYERLFKLTNTVCHQLGHYHHLNKDKQPQQVEDNGGYNNSNPESISKLQIESDMRELVQLVLNSSDGMDSNIKQTFLAVTKSFYYTAFTHPGTVNYHIAKVLFERVV

SEQ ID NO: 9 - protein, 792 aa, Euphobia peplus
MQVSLSLTTGSEPCITRIHAPSDAPLKQRNNEREKGTLELNGKVSLKKMGEMLRTIENVPIVGSTSSYDTAWVGMVPCSSNSSKPLFPESLKWIMENQNPEGNWAVDHAHHPLLLKDSLSSTLACVLALHKWNLAPQLVHSGLDFIGSNLWAAMDFRQRSPLGFDVIFPGMIHQAIDLGINLPFNNSSIENMLTNPLLDIQSFEAGKTSHIAYFAEGLGSRLKDWEQLLQYQTSNGSLFNSPSTTAAAAIHLRDEKCLNYLHSLTKQFDNGAVPTLYPLDARTRISIIDSLEKFGIHSHFIQEMTILLDQIYSFWKEGNEEIFKDPGCCATAFRLLRKHGYDVSSDSLAEFEKKEIFYHSSAASAHEIDTKSILELFRASQMKILQNEPILDRIYDWTSIFLRDQLVKGLIENKSLYEEVNFALGHPFANLDRLEARSYIDNYDPYDVPLLKTSYRSSNIDNKDLWTIAFQDFNKCQALHRVELDYLEKWVKEYKLDTLKWARQKTEYALFTIGAILSEPEYADARISWSQNTVFVTIVDDFFDYGGSLDECRNLINLMHKWDDHLTVGFLSEKVEIVFYSMYGTLNDLAAKAEVRQGRCVRSHLVNLWIWVMENMLKEREWADYNLVPTFYEYVAAGHITIGLGPVLLIALYFMGYPLSEDVVQSQEYKGVYLNVSIIARLLNDRVTVKRESAQGKLNGVSLFVEHGRGAVDEETSMKEVERLVESHKRELLRLIVQKTEGSVVPQSCKDLAWRVSKVLHLLYMDDDGFTCPVKMLNATNAIVNEPLLLTS

SEQ ID NO: 10 - protein, 782 aa, Euphobia peplus
MLLASSTSSRFFTKEWEPSNKTFSGSVRAQLSQRVKNIVVTPDQVKESESSGTSLRLKEMLKKVEMPISSYDTAWVAMVPSMEHSRNKPLFPNSLKWVMENQQPDGSWCFDDSNHPWLIKDSLSSTLASVLALKKWNVGQQLIDKGLEYIGSNMWAATDMHQYSPIGFNIIFPSMVEHANKLGLSLSLDHSLFQSMLRNRDMETKSLNGRNMAYVAEGLNGSNNWKEVMKYQRRNGSILNSPATTAAALIHLNDVKCFEYLDSLLTKFQHAVPTLYPFDIYARLCILDELEKLGVDRFVEIEKMLLLDYIYRCWLEGSEEILEDPTCCAMAFRFLRMNGYVVSPDVLQGFEEEEKLFHVKDTKSVLELLKASQLKVSEKEGILDRIYSWATSYLKHQLFNASISDKSLQNEVDYVVKHPHAILRRIENRNYIENYNTKNVSLRKTSFRFVNVDKRSDLLAHSRQDFNKCQIQFKKELAYLSRWEKKYGLDKLKYARQRLEVVYFSIASNLFEPEFSDARLAWTQYAILTTVVDDFFEYAASMDELVNLTNLIERWDEHGSEEFKSKEVEILFYAIYDLVNEDAEKAKKYQGRCIKSHLVHIWIDILKAMLKESEYVRYNIVPTLDEYISNGCTSISFGAILLIPLYFLGKMSEEVVTSKEYQKLYMHISMLGRLLNDRVTSQKDMAQGKLNSVSLRVLHSNGTLTEEEAKEEVDKIIEKHRRELLRMVVQTEGSVVPKACKKLFWMTSKELHLFYMTEDCFTCPTKLLSAVNSTLKDPLLMP

SEQ ID NO: 11 - protein, 575 aa, Salvia scarea
MSLAFNVGVTPFSGQRVGSRKEKFPVQGFPVTTPNRSRLIVNCSLTTIDFMAKMKENFKREDDKFPTTTTLRSEDIPSNLCIIDTLQRLGVDQFFQYEINTILDNTFRLWQEKHKVIYGNVTTHAMAFRLLRVKGYEVSSEELAPYGNQEAVSQQTNDLPMIIELYRAANERIYEEERSLEKILAWTTIFLNKQVQDNSIPDKKLHKLVEFYLRNYKGITIRLGARRNLELYDMTYYQALKSTNRFSNLCNEDFLVFAKQDFDIHEAQNQKGLQQLQRWYADCRLDTLNFGRDVVIIANYLASLIIGDHAFDYVRLAFAKTSVLVTIMDDFFDCHGSSQECDKIIELVKEWKENPDAEYGSEELEILFMALYNTVNELAERARVEQGRSVKEFLVKLWVEILSAFKIELDTWSNGTQQSFDEYISSSWLSNGSRLTGLLTMQFVGVKLSDEMLMSEECTDLARHVCMVGRLLNDVCSSEREREENIAGKSYSILLATEKDGRKVSEDEAIAEINEMVEYHWRKVLQIVYKKESILPRRCKDVFLEMAKGTFYAYGINDELTSPQQSKEDMKSFVF

SEQ ID NO: 12 - protein, 598 aa, Coleus forskohlii
MSSLAGNLRVIPFSGNRVQTRTGILPVHQTPMITSKSSAAVKCSLTTPTDLMGKIKEVFNREVDTSPAAMTTHSTDIPSNLCIIDTLQRLGIDQYFQSEIDAVLHDTYRLWQLKKKDIFSDITTHAMAFRLLRVKGYEVASDELAPYADQERINLQTIDVPTVVELYRAAQERLTEEDSTLEKLYVWTSAFLKQQLLTDAIPDKKLHKQVEYYLKNYHGILDRMGVRRNLDLYDISHYKSLKAAHRFYNLSNEDILAFARQDFNISQAQHQKELQQLQRWYADCRLDTLKFGRDVVRIGNFLTSAMIGDPELSDLRLAFAKHIVLVTRIDDFFDHGGPKEESYEILELVKEWKEKPAGEYVSEEVEILFTAVYNTVNELAEMAHIEQGRSVKDLLVKLWVEILSVFRIELDTWTNDTALTLEEYLSQSWVSIGCRICILISMQFQGVKLSDEMLQSEECTDLCRYVSMVDRLLNDVQTFEKERKENTGNSVSLLQAAHKDERVINEEEACIKVKELAEYNRRKLMQIVYKTGTIFPRKCKDLFLKACRIGCYLYSSGDEFTSPQQMMEDMKSLVYEPLPISPPEANNASGEKMSCVSN

SEQ ID NO: 13 - protein, 587 aa, Coleus forskohlii
MSITINLRVIAFPGHGVQSRQGIFAVMEFPRNKNTFKSSFAVKCSLSTPTDLMGKIKEKLSEKVDNSVAAMATDSADMPTNLCIVDSLQRLGVEKYFQSEIDTVLDDAYRLWQLKQKDIFSDITTHAMAFRLLRVKGYDVSSEELAPYADQEGMNLQTIDLAAVIELYRAAQERVAEEDSTLEKLYVWTSTFLKQQLLAGAIPDQKLHKQVEYYLKNYHGILDRMGVRKGLDLYDAGYYKALKAADRLVDLCNEDLLAFARQDFNINQAQHRKELEQLQRWYADCRLDKLEFGRDVVRVSNFLTSAILGDPELSEVRLVFAKHIVLVTRIDDFFDHGGPREESHKILELIKEWKEKPAGEYVSKEVEILYTAVYNTVNELAERANVEQGRNVEPFLRTLWVQILSIFKIELDTWSDDTALTLDDYLNNSWVSIGCRICILMSMQFIGMKLPEEMLLSEECVDLCRHVSMVDRLLNDVQTFEKERKENTGNAVSLLLAAHKGERAFSEEEAIAKAKYLADCNRRSLMQIVYKTGTIFPRKCKDMFLKVCRIGCYLYASGDEFTSPQQMMEDMKSLVYEPLQIHPPAAA

SEQ ID NO: 14 - protein, 733 aa, Tripterygium Wilfordii
MFDKTQLSVSAYDTAWVAMVSSPNSRQAPWFPECVNWLLDNQLSDGSWGLPPHHPSLVKDALSSTLACLLALKRWGLGEQQMTKGLQFIESNFTSINDEEQHTPIGFNIIFPGMIETAIDMNLNLPLRSEDINVMLHNRDLELRRNKLEGREAYLAYVSEGMGKLQDWEMVMKYQRKNGSLFNSPSTTAAALSHLGNAGCFHYINSLVAKFGNAVPTVYPSDKYALLCMIESLERLGIDRHFSKEIRDVLEETYRCWLQGDEEIFSDADTCAMAFRILRVHGYEVSSDPLTQCAEHHFSRSFGGHLKDFSTALELFKASQFVIFPEESGLEKQMSWTNQFLKQEFSNGTTRADRFSKYFSIEVHDTLKFPFHANVERLAHRRNIEHHHVDNTRILKTSYCFSNISNADFLQLAVEDFNRCQSIHREELKHLERWVVETKLDRLKFARQKMAYCYFSAAGTCFSPELSDARISWAKNSVLTTVADDFFDIVGSEEELANLVHLLENWDANGSPHYCSEPVEIIFSALRSTICEIGDKALAWQGRSVTHHVIEMWLDLLKSALREAEWARNKVVPTFDEYVENGYVSMALGPIVLPAVYLIGPKVSEEVVRSPEFHNLFKLMSICGRLINDTRTFKRESEAGKLNSVLLHMIHSGSGTTEEEAVEKIRGMIADGRRELLRLVLQEKDSVVPRACKDLFWKMVQVLHLFYMDGDGFSSPDMMLNAVNALIREPISL

SEQ ID NO: 15 - protein, 783 aa, Euphobia peplus
MSATPNSFFTSPISAKLGHPKSQSVAESNTRIQQLDGTREKIKKMFDKVELSVSPYDTAWVAMVPSPNSLEAPYFPECSKWIVDNQLNDGSWGVYHRDPLLVKDSISSTLACVLALKRWGIGEKQVNKGLEFIELNSASLNDLKQYKPVGFDITFPRMLEHAKDFGLNLPLDPKYVEAVIFSRDLDLKSGCDSTTEGRKAYLAYISEGIGNLQDWNMVMKYQRRNGSIFDSPSATAAASIHLHDASCLRYLRCALKKFGNAVPTIYPFNIYVRLSMVDAIESLGIARHFQEEIKTVLDETYRYWLQGNEEIFQDCTTCAMAFRILRANGYNVSSEKLNQFTEDHFSNSLGGYLEDMRPVLELYKASQLIFPDELFLEKQFSWTSQCLKQKISSGLRHTDGINKHITEEVNDVLKFASYADLERLTNWRRIAVYRANETKMLKTSYRCSNIANEHFLELAVEDFNVCQSMHREELKHLGRWVVEKRLDKLKFARQKLGYCYFSSAASLFAPEMSDARISWAKNAVLTTVVDDFFDVGGSEEELINLVQLIERWDVDGSSHFCSEHVEIVFSALHSTICEIGEKAFAYQGRRMTSHVIKIWLDLLKSMLTETLWSKSKATPTLNEYMTNGNTSFALGPIVLPALFFVGPKLTDEDLKSHELHDLFKTMSTCGRLLNDWRSYERESEEGKLNAVSLHMIYGNGSVAATEEEATQKIKGLIESERRELMRLVLQEKDSKIPRPCKDLFWKMLKVLHMFYLKDDGFTSNQMMKTANSLINQPISLHER

SEQ ID NO: 16 - protein, 792 aa, Coleus forskohlii
MSLPLSTCVLFVPKGSQFWSSRFSYASASLEVGFQRATSAQIAPLSKSFEETKGRIAKLFHKDELSISTYDTAWVAMVPSPTSSEEPCFPACLNWLLENQCLDGSWARPHHHPMLKKDVLSSTLACILALKKWGVGEEQINRGLHFIELNFASATEKCQITPMGFDIVFPAMLDRARALSLNIRLEPTTLNDLMNKRDLELNRCYQSSSTEREVYRAYIAEGMGKLQNWESVMKYQRKNGTLFNCPSTTAAAFTALRNSDCLNYLHLALNKFGDAVPAVFPLDIYSQLCIVDNLERVGISRHFLTEIQSVLDGTYRSWLQGDEQIFMDASTCALAFRTLRMNGYNVSSDPITKLIQEGSFSRNTMDINTTLELYRASELILYPDERDLEEHNLRLKTILDQELSGGGFILSRQLGRNINAEVKQALESPFYAIMDRMAKRRSIEHYHIDNTRILKTSYCSPNFGNEDFLSLSVEDFNRCQVIHREELRELERWVIENRLDELKFARSKSAYCYFSAAATIFSPELSDARMSWAKNGVLTTVVDDFFDVGGSVEELKNLIQLVELWDVDVSRECISPSVQIIFSALKHTIREIGDKGFKLQGRSITDHIIAIWLDLLYSMMKESEWGREKAVPTIDEYISNAYVSFALGPIVLPALYLVGPKLSEEMVNHADYHNLFKSMSTCGRLLNDIRGYERELKDGKLNTLSLYMVNNEGEISWEAAILEVKSWIERERRELLRSVLEEEKSVVPKACKELFWHMCTVVHLFYSKDDGFTSQDLLSAVNAIIYQPLVLE

SEQ ID NO: 17 - protein, 773 aa, Coleus forskohlii
MKMLMIKSQFRVHSIVSAWANNSNKRQSLGHQIRRKQRSQVTECRVASLDALNGIQKVGPATIGTPEEENKKIEDSIEYVKELLKTMGDGRISVSPYDTAIVALIKDLEGGDGPEFPSCLEWIAQNQLADGSWGDHFFCIYDRVVNTAACVVALKSWNVHADKIEKGAVYLKENVHKLKDGKIEHMPAGFEFVVPATLERAKALGIKGLPYDDPFIREIYSAKQTRLTKIPKGMIYESPTSLLYSLDGLEGLEWDKILKLQSADGSFITSVSSTAFVFMHTNDLKCHAFIKNALTNCNGGVPHTYPVDIFARLWAVDRLQRLGISRFFEPEIKYLMDHINNVWREKGVFSSRHSQFADIDDTSMGIRLLKMHGYNVNPNALEHFKQKDGKFTCYADQHIESPSPMYNLYRAAQLRFPGEEILQQALQFAYNFLHENLASNHFQEKWVISDHLIDEVRIGLKMPWYATLPRVEASYYLQHYGGSSDVWIGKTLYRMPEISNDTYKILAQLDFNKCQAQHQLEWMSMKEWYQSNNVKEFGISKKELLLAYFLAAATMFEPERTQERIMWAKTQVVSRMITSFLNKENTMSFDLKIALLTQPQHQINGSEMKNGLAQTLPAAFRQLLKEFDKYTRHQLRNTWNKWLMKLKQGDDNGGADAELLANTLNICAGHNEDILSHYEYTALSSLTNKICQRLSQIQDKKMLEIEEGSIKDKEMELEIQTLVKLVLQETSGGIDRNIKQTFLSVFKTFYYRAYHDAKTIDAHIFQVLFEPVV

SEQ ID NO: 18 - protein, 580 aa, Marrubium vulgare
MSITFNLKIAPFSGPGIQRSKETFPATEIQITASTKSTMTTKCSFNASTDFMGKLREKVGGKADKPPVVIHPVDISSNLCMIDTLQSLGVDRYFQSEINTLLEHTYRLWKEKKKNIIFKDVSCCAIAFRLLREKGYQVSSDKLAPFADYRIRDVATILELYRASQARLYEDEHTLEKLHDWSSNLLKQHLLNGSIPDHKLHKQVEYFLKNYHGILDRVAVRRSLDLYNINHHHRIPDVADGFPKEDFLEYSMQDFNICQAQQQEELHQLQRWYADCRLDTLNYGRDVVRIANFLTSAIFGEPEFSDARLAFAKHIILVTRIDDFFDHGGSREESYKILDLVQEWKEKPAEEYGSKEVEILFTAVYNTVNDLAEKAHIEQGRCVKPLLIKLWVEILTSFKKELDSWTEETALTLDEYLSSSWVSIGCRICILNSLQYLGIKLSEEMLSSQECTDLCRHVSSVDRLLNDVQTFKKERLENTINSVGLQLAAHKGERAMTEEDAMSKIKEMADYHRRKLMQIVYKEGTVFPRECKDVFLRVCRIGYYLYSSGDEFTSPQQMKEDMKSLVYQPVKIHPLEAINV

SEQ ID NO: 19 - DNA, 2361 bp, Coleus forskohlii
atgggttccttgtctaccatgaacttgaaccattctccaatgtcctactctggtattttgccatcttcttcagctaaggctaagttgttgttgccaggttgtttttctatttccgcttggatgaacaacggtaagaatttgaattgccaattgacccacaagaagatctctaaggttgccgaaattagagttgctactgttaatgctccaccagttcatgatcaagatgactctactgaaaatcaatgccatgatgccgttaacaacatcgaagatccaatcgaatatatcagaaccttgttgagaactaccggtgatggtagaatttctgtttctccatatgatactgcttgggtcgctttgattaaggacttgcaaggtagagatgctccagaatttccatcttcattggaatggatcatccaaaatcaattggctgatggttcttggggtgatgctaagtttttttgcgtttacgatagattggtcaacaccattgcttgtgttgttgctttgagatcttgggatgttcatgctgaaaaagttgaaagaggtgtcagatatatcaacgaaaacgtcgaaaagttgagagatggtaacgaagaacatatgacctgtggtttcgaagttgttttcccagctttgttgcaaagagctaagtctttgggtattcaagatttgccatatgatgccccagttatccaagaaatctatcactctagagaacaaaagtccaagagaatcccattggaaatgatgcataaggtcccaactagtttgttgttctctttggaaggtttggaaaacttggaatgggacaagttgttgaagttgcaatcagcagatggttcctttttgacttctccatcttctactgctttcgctttcatgcaaactagagatccaaagtgctaccaattcatcaagaacaccattcaaactttcaacggtggtgctccacatacttatccagttgatgtttttggtagattgtgggccattgacagattgcaaagattgggtatttccagattcttcgaatccgaaattgctgactgcattgcccatattcatagattctggactgaaaagggtgttttctctggtagagaatctgaattctgcgatatcgatgatacctctatgggtgttagattgatgagaatgcatggttacgatgttgatccaaacgtcttgaagaatttcaagaaggacgataagttctcttgctacggtggtcaaatgattgaatctccatctccaatctacaacttgtacagagcttcccaattgagatttccaggtgaacaaattttggaagatgccaacaagttcgcctacgactttttacaagaaaagttggcccataatcaaatcttggacaagtgggttatttccaaacatttgccagacgaaatcaagttgggtttagaaatgccatggtatgctactttgccaagagttgaagccagatattacatccaatattacgctggttctggtgatgtttggattggtaaaaccttgtatagaatgccagaaatctccaacgatacctatcatgaattggctaagaccgatttcaagagatgtcaagctcaacatcaatttgaatggatctacatgcaagaatggtacgaatcttgcaacatggaagaattcggtatctccagaaaagaattattggtcgcttacttcttggctaccgcttctatttttgaattggaaagagccaacgaaagaattgcttgggctaagtctcaaatcatctctactattatcgcctccttcttcaacaatcaaaacacctctccagaagataagttggctttcttgactgactttaagaacggtaactctaccaacatggctttggttactttgacccaattcttagaaggtttcgacagatacacttcccaccaattgaaaaatgcttggtctgtttggttgagaaagttgcaacaaggtgaaggtaatggtggtgctgatgctgaattattagttaacaccttgaacatttgcgccggtcatattgctttcagagaagaaattttggctcacaacgattacaagaccttgtctaacttgacctctaagatctgcagacaattgagtcaaatccaaaacgaaaaagaattggaaaccgaaggtcaaaagacctccattaagaacaaagaattagaagaagatatgcaaagattagtcaagttggtcttggaaaagtccagagttggtatcaacagagacatgaagaaaactttcttggccgttgttaagacctactactacaaagcttatcattccgctcaagccatcgataaccatatgtttaaggttttgttcgaaccagtcgcctga

SEQ ID NO: 20 - DNA, 1704 bp, Coleus forskohlii
atgatcacctccaaatcttccgctgctgttaagtgttctttgactactccaactgatttgatgggtaagatcaaagaagttttcaacagagaagttgatacctctccagctgctatgactactcattctactgatattccatccaacttgtgcatcatcgataccttgcaaagattgggtatcgaccaatacttccaatccgaaattgatgctgtcttgcatgatacttacagattgtggcaattgaagaagaaggacatcttctctgatattaccactcatgctatggccttcagattattgagagttaagggttacgaagttgcctctgatgaattggctccatatgctgatcaagaaagaatcaacttgcaaaccattgatgttccaaccgtcgtcgaattatacagagctgcacaagaaagattgaccgaagaagattctaccttggaaaagttgtacgtttggacttctgctttcttgaagcaacaattattgaccgatgccatcccagataagaagttgcataagcaagtcgaatattacttgaagaactaccacggtatcttggatagaatgggtgttagaagaaacttggacttgtacgatatctcccactacaaatctttgaaggctgctcatagattctacaacttgtctaacgaagatattttggccttcgccagacaagatttcaacatttctcaagcccaacaccaaaaagaattgcaacaattgcaaagatggtacgccgattgcagattggatactttgaaattcggtagagatgtcgtcagaatcggtaactttttaacctctgctatgatcggtgatccagaattgtctgatttgagattggcttttgctaagcacatcgttttggttaccagaatcgatgatttcttcgatcatggtggtccaaaagaagaatcctacgaaattttggaattggtcaaagaatggaaagaaaagccagctggtgaatacgtttctgaagaagtcgaaatcttattcaccgctgtttacaacaccgttaacgaattggctgaaatggcccatattgaacaaggtagatctgttaaggatttgttggttaagttgtgggtcgaaatattgtccgttttcagaatcgaattggatacctggactaacgatactgctttgactttggaagaatacttgtcccaatcctgggtttctattggttgcagaatctgcattttgatctccatgcaattccaaggtgttaagttgagtgacgaaatgttgcaaagtgaagaatgtaccgatttgtgcagatacgtttccatggtcgatagattattgaacgatgtccaaaccttcgaaaaagaaagaaaagaaaacaccggtaactccgtttctttgttgcaagctgctcacaaagacgaaagagttatcaacgaagaagaagcctgcatcaaggtaaaagaattagccgaatacaatagaagaaagttgatgcaaatcgtctacaagaccggtactattttcccaagaaaatgcaaggacttgttcttgaaggcttgtagaattggttgctacttgtactcttctggtgatgaattcacttccccacaacaaatgatggaagatatgaagtccttggtctatgaaccattgccaatttctccacctgaagctaacaatgcatctggtgaaaaaatgtcctgcgtcagtaactga

SEQ ID NO: 21 - DNA, 2361 bp, Zea Maiz
atggcccaacatacttctgaatctgctgctgttgctaaaggttcttctttgactccaatcgttagaaccgatgctgaatctagaagaactagatggccaacagatgatgatgacgctgaaccattggttgacgaaattagagctatgttgacctctatgtccgatggtgatatttctgtttctgcttatgatactgcttgggttggtttggttccaagattggatggtggtgaaggtccacaatttccagctgctgttagatggattagaaacaatcaattgccagatggttcttggggtgatgctgctttgttttcagcttacgatagattgattaacaccttggcttgtgttgttactttgaccagatggtctttggaaccagaaatgagaggtagaggtttgtcttttttgggtagaaacatgtggaagttggctaccgaagatgaagaatctatgccaattggtttcgaattggctttcccatccttgattgaattggctaaatctttgggtgttcacgatttcccatatgatcatcaagctttacaaggtatctactcctccagagaaatcaaaatgaagagaatcccaaaagaagtcatgcatactgttccaacctctatcttgcattctttggaaggtatgccaggtttggattgggctaagttgttgaaattgcaatcctctgatggttcattcttgttttcaccagctgctactgcttacgctttgatgaatactggtgatgatagatgcttctcctacattgatagaaccgtcaaaaagttcaatggtggtgttccaaatgtttacccagttgacttgtttgaacatatctgggctgttgacagattggaaagattgggtatttccagatacttccaaaaagaaatcgaacaatgcatggactacgttaacagacattggactgaagatggtatttgttgggctagaaactccgacgtaaaagaagttgacgatactgctatggccttcagattattgagattgcatggttactctgtttccccagatgttttcaagaacttcgaaaaggatggtgaattcttcgctttcgtcggtcaatctaatcaagctgttactggtatgtacaacttgaacagagcctcccaaatttcatttccaggtgaagatgttttacacagagctggtgctttttcttacgaattcttgagaagaaaagaagccgaaggtgctttgagagataagtggattatttccaaggatttgcctggtgaagttgtctacactttggattttccatggtacggtaatttgccaagagttgaagctagagactacttggaacaatatggtggtggtgatgacgtttggataggtaaaacattatacagaatgccattggtcaacaacgacgtttatttggaattggccagaatggatttcaaccattgtcaagccttgcatcaattggaatggcaaggtttgaaaagatggtacaccgaaaacagattgatggattttggtgttgctcaagaagatgcattgagagcttactttttggctgctgcttcagtttatgaaccatgtagagctgctgaaagattagcttgggcaagagctgctattttggctaatgctgtttctactcacttgagaaactctccatctttcagagaaagattggaacactctttgagatgcagaccttctgaagaaactgatggtagttggttcaattcctcttctggttctgatgctgttttggttaaggcagttttgagattgactgattccttggctagagaagctcaacctattcacggtggtgatccagaagatattattcacaagttgttaagatccgcttgggctgaatgggttagagaaaaagctgatgctgcagattctgtctgtaatggttcttctgctgttgaacaagaaggttccagaatggttcatgataagcaaacctgtttgttgttggcaagaatgattgaaatttccgctggtagagccgctggtgaagctgcttccgaagatggtgacagaagaattatacaattgaccggttccatctgcgactcattgaaacaaaaaatgttggtcagtcaagacccagaaaagaacgaagaaatgatgtcccatgttgacgacgaattgaagttgagaatcagagaattcgtccaatacttgttgagattgggtgaaaaaaagactggttcctctgaaaccagacaaactttcttgtctatcgtcaagtcttgttactacgctgctcattgtccaccacatgttgttgatagacatatctccagagttatcttcgaaccagtttctgctgctaaattggaacatcatcaccatcaccactga

SEQ ID NO: 22 - DNA, 2316 bp, Euphobia peplus
atggctcaatccgttgctgaatccaacaccagaattcaacaattggatggtactagagaaaagatcaagaagatgttcgacaaggtcgaattgtctgtttctccatatgatactgcttgggttgctatggttccatctccaaattctttggaagctccatactttccagaatgctctaaatggatcgtcgacaatcaattgaatgatggttcttggggtttctaccatagagatccattattggttaaggactccatctcttctactttggcttgtgttttggctttgaaaagatggggtattggtgaaaagcaagtcaacaaaggtttggaattcatcgaattgaactccgcctctttgaacgatttgaaacaatacaagccagtcggtttcgatattacctttccaagaatgttggaacacgctaaggatttcggtttgaatttgccattggatcctaagtatgttgaagccgttatcttctccagagatttggatttgaaatccggttgtgattctactaccgaaggtagaaaagcttacttggcctatatttccgaaggtatcggtaacttgcaagattggaatatggtcatgaagtaccaaagaagaaacggttccattttcgattctccatctgctacagctgctgcttctattcacttgcatgatgcttcatgtttgagatacttgagatgcgccttgaagaaatttggtaatgctgttccaactatctacccattcaacatctacgtcagattgtctatggttgatgccattgaatctttgggtattgccagacactttcaagaagaaatcaagaccgttttggacgaaacttacagatattggttgcaaggtaacgaagaaatcttccaagattgcactacttgtgctatggccttcagaattttgagagctaatggttacaacgtttcctccgaaaagttgaatcaattcaccgaagatcacttctccaattcattgggtggttatttggaagatatgagaccagtcttggaattatacaaggcctcccaattgattttcccagacgaattattcttagaaaagcaattctcctggacctcccaatgtttgaagcaaaaaatctcttccggtttgagacataccgacggtattaacaaacacattaccgaagaagttaacgacgttttgaagttcgcttcttacgctgatttggaaagattgaccaattggagaagaatcgctgtttacagagctaacgaaacaaaaatgttgaaaacctcctacagatgctccaacattgctaacgaacactttttggaattggccgtcgaagatttcaacgtttgtcaatcaatgcacagagaagaattgaagcacttgggtagatgggttgttgaaaagagattggacaagttgaaattcgccagacaaaagttgggttactgctacttttcttcagctgcttctttgtttgctccagaaatgtctgatgctagaatttcttgggctaagaatgccgttttgactaccgttgttgatgacttttttgatgtcggtggttccgaagaagaattgattaacttggtccaattgatcgaaagatgggacgttgatggttcctctcatttctgttctgaacatgtcgaaatcgttttctctgccttgcattctaccatttgcgaaataggtgaaaaggcttttgcttatcaaggtagaagaatgacctcccacgttattaagatttggttggacttgttgaagtccatgttgactgaaactttgtggtctaagtctaaggctactccaaccttgaacgaatatatgactaacggtaacacctcttttgctttgggtccaatagttttgccagctttgttttttgttggtccaaagttgaccgacgaagatttgaagtctcatgaattgcacgatttgttcaagaccatgtctacctgtggtagattattgaacgattggagatcctacgaaagagaatctgaagaaggtaaattgaacgccgtttccttgcatatgatctacggtaatggttctgttgctgctactgaagaagaagctactcaaaagattaagggtttgatcgaatccgaaagaagagaattgatgagattggtattgcaagaaaaggactctaagattcctagaccatgcaaggatttgttctggaagatgttgaaggtcttgcacatgttctacttgaaggatgatggtttcacctccaatcaaatgatgaagactgctaactccttgatcaatcaacctatctcattgcacgaaagagttgaacatcatcatcaccatcactaa

SEQ ID NO: 23 - DNA, 2328 bp, Tripterygium Wilfordii
atgggtatcgctaaatccaagccagctagaactactccagaatactctgatgttttacaaactggtttgccattgatcgtcgaagatgatatccaagaacaagaagaaccattggaagtttctttggaaaatcaaatcagacaaggtgtcgacatcgtcaaatctatgttgggttctatggaagatggtgaaacctctatttctgcttatgatactgcttgggttgccttggttgaaaacattcatcatccaggtagtccacaattcccatcttcattacaatggatcgccaacaatcaattgccagatggttcttggggtgatccagatgtttttttggctcatgatagattgattaacaccttggcttgcgttattgctttgaagaagtggaatatccatccacacaaatgcaagagaggtttgtctttcgtcaaagaaaacatttctaagttggaaaaagaaaacgaagaacacatgttgatcggtttcgaaattgcctttccatccttgttggaaatggctaagaaattgggtatcgaaatcccagatgattctccagctttacaagatatctacaccaagagagatttgaagttgaccagaatcccaaaggataagatgcataacgttccaactaccttgttgcattcattggaaggtttgccagatttggattgggaaaagttggttaagttgcaattccaaaacggttcctttttgttctctccatcttctactgcttttgcctttatgcataccaaggatggtaactgcttgtcctacttgaatgatttggttcacaagttcaatggtggtgttccaactgcttatccagttgatttgtttgaacacatctggtccgttgacagattgcaaagattgggtatttccagattcttccacccagaaatcaaagaatgtttgggttacgttcatagatactggactaaggacggtatttgttgggctagaaattccagagttcaagatattgatgataccgccatgggtttcagattattgagattgcatggttacgaagtttccccagatgtctttaagcaattcagaaagggtgatgaattcgtctgtttcatgggtcaatccaatcaagctattaccggtatctacaacttgtacagagcttcccaaatgatgttcccagaagaaaccattttggaagaagccaagaagttctccgttaacttcttgagagaaaagagagctgcctctgaattattggataagtggattatcaccaaggacttgccaaatgaagttggttttgctttggatgttccatggtatgcttgtttgccaagagttgaaaccagattgtacatcgaacaatacggtggtcaagatgatgtttggataggtaagaccttgtatagaatgccatacgtcaacaacaacgtctacttggaattggccaaattggattacaacaactgccaatccttgcacagaattgaatgggacaatatccaaaagtggtacgaaggttacaatttgggtggttttggtgtcaacaagagatccttattgagaacctactttttggccacctccaacatttttgaaccagaaagatctgtcgaaagattgacttgggctaagactgctattttggttcaagccattgcttcctacttcgaaaactctagagaagaaagaatcgaattcgccaacgaatttcaaaagttcccaaacactagaggttacatcaacggtagaagattggatgttaagcaagctaccaagggtttgatcgaaatggttttcgctaccttgaatcaattctccttggatgccttagttgttcacggtgaagatattactcatcacttgtaccaatcctgggaaaaatgggttttgacttggcaagaaggtggtgatagaagagaaggtgaagccgaattattagtccaaaccattaacttgatggccggtcatactcatagtcaagaagaagaattatacgaaagattattcaagttgactaacaccgtctgccatcaattgggtcattatcatcatttgaacaaggataagcaaccacaacaagtcgaagataatggtggttacaacaattccaacccagaatccatctccaagttgcaaattgaatccgacatgagagaattggtccaattggttttgaactcctctgatggtatggactctaacatcaagcaaactttcttggctgttaccaagtctttctactacactgcttttactcatcctggtactgtcaactaccatattgctaaggttttgttcgaaagagtcgtcttagaacatcatcatcaccatcactga

SEQ ID NO: 24 - DNA, 1728 bp, Salvia scarea
atgtccttggctttcaacgttggtgttactccattttctggtcaaagagtcggttccagaaaagaaaagtttccagttcaaggtttcccagttactactccaaatagatccagattgatcgtcaactgttccttgactaccattgatttcatggccaagatgaaggaaaacttcaagagagaagatgacaagttcccaactactactaccttgagatctgaagatatcccatccaacttgtgcattatcgataccttgcaaagattgggtgttgaccaattcttccaatacgaaatcaacaccatcttggacaacactttcagattgtggcaagaaaagcacaaggttatctacggtaatgttactacacatgctatggccttcagattattgagagttaagggttacgaagtttcctccgaagaattagctccatacggtaatcaagaagccgtttctcaacaaactaacgacttgccaatgatcatcgaattatacagagctgccaacgaaagaatctacgaagaagaaagatccttggaaaagattttggcttggaccaccattttcttgaacaagcaagttcaagacaactccatcccagataagaagttgcataagttggtcgaattctacttgagaaactacaagggtatcaccattagattaggtgccagaagaaacttggaattatacgacatgacttactaccaagccttgaagtctaccaacagattctctaacttgtgtaacgaagatttcttggttttcgccaagcaagatttcgatattcacgaagcccaaaatcaaaagggtttacaacaattacaaagatggtacgccgattgcagattggatactttgaatttcggtagagatgtcgtcattatcgctaactatttggcctccttgattattggtgatcatgcctttgattacgtcagattggcttttgctaagacctctgttttggttaccatcatggatgatttcttcgattgccatggttcttctcaagaatgcgacaagataatcgaattggtaaaagaatggaaagaaaacccagatgccgaatacggttctgaagaattggaaattttgttcatggccttgtacaacaccgttaacgaattggctgaaagagctagagttgaacaaggtagatctgtcaaagaatttttggtcaagttgtgggttgaaatcttgtccgctttcaagattgaattggatacctggtctaacggtactcaacaatctttcgacgaatatatctcctcctcttggttgtctaatggttctagattgactggtttgttgaccatgcaatttgttggtgtcaaattgtccgacgaaatgttgatgtcagaagaatgtactgatttggctagacacgtatgtatggtcggtagattattgaacgatgtctgctcatctgaaagagaaagagaagaaaacattgccggtaagtcctactctattttgttggctactgaaaaggacggtagaaaggtttctgaagatgaagctattgctgaaatcaacgaaatggtcgaataccattggagaaaggtcttgcaaatcgtctacaagaaagaatccatcttgcctagaagatgcaaggacgtttttttggaaatggctaagggtactttttacgcctacggtattaacgatgaattgacctctccacaacaatccaaagaagatatgaagtccttcgttttttaa

SEQ ID NO: 25 - DNA, 2448 bp, Tripterygium Wilfordii
atgtttatgtcctcctcctcatcctctcatgctagaagaccacaattgtcatctttctcttacttgcatccaccattgccatttccaggtttgtcatttttcaacaccagagacaagagagtcaacttcgattctaccagaattatctgcattgccaaatctaagccagctagaactactccagaatactccgatgttttacaaactggtttgccattgatcgtcgaagatgatatccaagaacaagaagaaccattggaagtttctttggaaaatcaaatcagacaaggtgtcgacatcgtcaaatctatgttgggttctatggaagatggtgaaacctctatttctgcttatgatactgcttgggttgccttggttgaaaacattcatcatccaggtagtccacaattcccatcttcattacaatggatcgccaacaatcaattgccagatggttcttggggtgatccagatgtttttttggctcatgatagattgattaacaccttggcttgcgttattgctttgaagaagtggaatatccatccacacaaatgcaagagaggtttgtctttcgtcaaagaaaacatttctaagttggaaaaagaaaacgaagaacacatgttgatcggtttcgaaattgcctttccatccttgttagaaatggctaagaagttgggtatcgaaatcccagatgattctccagctttacaagatatctacaccaagagagatttgaagttgaccagaatcccaaaggatatcatgcataacgttccaactaccttgttgtactctttggaaggtttgccttctttggattgggaaaagttggttaagttgcaatgtactgacggttcctttttgttctctccatcttctactgcttgtgctttgatgcatacaaaagatggtaactgcttctcctacatcaacaacttggtccataagtttaatggtggtgttccaactgtttacccagttgatttgtttgaacatatctggtgcgttgacagattgcaaagattgggtatttccagattcttccacccagaaatcaaagaatgtttgggttacgttcatagatactggaccaaggatggtatttgttgggctagaaattccagagttcaagatattgatgataccgccatgggtttcagattattgagattgcatggttacgaagtttccccagatgtctttaagcaattcagaaagggtgatgaattcgtctgtttcatgggtcaatccaatcaagctattaccggtatctacaacttgtacagagcttcccaaatgatgttcccagaagaaaccattttggaagaagccaagaagttctccgttaacttcttgagagaaaagagagctgcctctgaattattggataagtggattatcaccaaggacttgccaaatgaagttggttttgctttggatgttccatggtatgcttgtttgccaagagttgaaaccagattgtacatcgaacaatacggtggtcaagatgatgtttggataggtaagaccttgtatagaatgccatacgtcaacaacaacgtctacttggaattggccaaattggattacaacaactgccaatccttgcacagaattgaatgggacaatatccaaaagtggtacgaaggttacaatttgggtggttttggtgtcaacaagagatccttattgagaacctactttttggccacctccaacatttttgaaccagaaagatctgtcgaaagattgacttgggctaagactgctattttggttcaagccattgcttcctacttcgaaaactctagagaagaaagaatcgaattcgccaacgaattccaaaagttcccaaacactagaggttacatcaacggtagaagattggatgttaagcaagctaccaagggtttgatcgaaatggttttcgctaccttgaatcaattctccttggatgcattggttgttcacggtgaagatattactcatcacttgtaccaatcctgggaaaaatgggttttgacttggcaagaaggtggtgatagaagagaaggtgaagccgaattattagtccaaaccattaacttgatggccggtcatactcatagtcaagaagaagaattatacgaaagattattcaagttgactaacaccgtctgccatcaattgggtcattatcatcatttgaacaaggacaagcaaccacaacaagtcgaagataacggtggttacaacaattctaacccagaatccatctccaagttgcaaatcgaatctgacatgagagaattggtccaattggtcttgaattcctctgatggtatggactctaacatcaagcaaactttcttggctgttaccaagtctttctactacactgcttttactcatcctggtactgtcaactaccatattgctaaggttttgttcgaaagagttgtttaa

SEQ ID NO: 26 - protein, 2 aa, Coleus forskohlii
  misc_feature (1)..(2): Xaa can be any naturally occurring amino acid
XX

SEQ ID NO: 27 - protein, 2 aa, Coleus forskohlii
  misc_feature (1)..(2): Xaa can be any naturally occurring amino acid
XX

SEQ ID NO: 28 - protein, 776 aa, Marrubium vulgare
MASTPTLNLSITTPFVRTKIPAKISLPACSWLDRSSSRHVELNHKFCRKLELKVAMCRASLDVQQVRDEVYSNAQPHELVDKKIEERVKYVKNLLSTMDDGRINWSAYDTAWISLIKDFEGRDCPQFPSTLERIAENQLPDGSWGDKDFDCSYDRIINTLACVVALTTWNVHPEINQKGIRYLKENMRKLEETPTVLMTCAFEVVFPALLKKARNLGIHDLPYDMPIVKEICKIGDEKLARIPKKMMEKETTSLMYAAEGVENLDWERLLKLRTPENGSFLSSPAATVVAFMHTKDEDCLRYIKYLLNKFNGGAPNVYPVDLWSRLWATDRLQRLGISRYFESEIKDLLSYVHSYWTDIGVYCTRDSKYADIDDTSMGFRLLRVQGYNMDANVFKYFQKDDKFVCLGGQMNGSATATYNLYRAAQYQFPGEQILEDARKFSQQFLQESIDTNNLLDKWVISPHIPEEMRFGMEMTWYSCLPRIEASYYLQHYGATEDVWLGKTFFRMEEISNENYRELAILDFSKCQAQHQTEWIHMQEWYESNNVKEFGISRKDLLFAYFLAAASIFETERAKERILWARSKIICKMVKSFLEKETGSLEHKIAFLTGSGDKGNGPVNNAMATLHQLLGEFDGYISIQLENAWAAWLTKLEQGEANDGELLATTINICGGRVNQDTLSHNEYKALSDLTNKICHNLAQIQNDKGDEIKDSKRSERDKEVEQDMQALAKLVFEESDLERSIKQTFLAVVRTYYYGAYIAAEKIDVHMFKVLFKPVG

(19) United States
(12) Patent Application Publication
(10) Pub. No.: US 2018/0037912 A1
(43) Pub. Date: Feb. 8, 2018

(54) Methods for Producing Diterpenes

(71) Applicants: University of Copenhagen, Copenhagen (DK); Danmarks Tekniske Universitet, Lyngby (DK)

(72) Inventors: Bjorn Hamberger, Kastrup (DK); Birger Lindberg Moller, Bronshoj (DK); Johan Andersen-Ranberg, Copenhagen (DK); Carl Jorg Bohlmann, Vancouver, British Columbia (CA); Philipp Zerbe, North Vancouver, British Columbia (CA); Morten Thrane Nielsen, Copenhagen (DK)

(21) Appl. No.: 15/110,454

(22) PCT Filed: Jan. 30, 2015
(86) PCT No.: PCT/DK2015/050021

§ 371 Date: Jul. 8, 2016

(30) Foreign Application Priority Data
Jan. 31, 2014 (DK) PA 2014 00056

(51) Int. Cl. C12P 5/00; C07K 14/415; C12N 9/16; C12P 17/06; C12N 9/10

(52) U.S. Cl. CPC C12P 5/007; C12P 17/06; C12N 9/1051; C12Y 204/01015; C12N 9/16; C12Y 301/03012; C07K 14/415

(57) ABSTRACT

The present invention discloses that by combining different di TPS enzymes of class I and class II different diterpenes may be produced including diterpenes not identified in nature. Surprisingly it is revealed that a di TPS enzyme of class I of one species may be combined with a di TPS enzyme of class II from a different species, resulting in a high diversity of diterpenes, which can be produced.

### FIELD OF INVENTION

The present invention relates to the field of biosynthetic methods for producing diterpenes.

### BACKGROUND OF INVENTION

Terpenes constitute a large and diverse class of organic compounds produced by a variety of plants as well as other species. Terpenes modified by oxidation or rearrangements are generally referred to as terpenoids.

Terpenes and terpenoids find multiple uses, for example as flavor compounds, additives for food, as fragrances and in medical treatment

Terpenes are derived biosynthetically from units of isoprene, which has the molecular formula C₅H₈. Diterpenes are composed of four isoprene units and in nature they are produced from geranylgeranyl pyrophosphate.

### SUMMARY OF INVENTION

In nature diterpenes are produced with the aid of specific pairs of diterpene synthases (diTPS) derived from two classes, class I and class II.

The present invention discloses that by combining different diTPS enzymes of class I and class II different diterpenes may be produced including diterpenes not identified in nature. Surprisingly it is revealed that a diTPS enzyme of class I of one species may be combined with a diTPS enzyme of class II from a different species, resulting in a high diversity of diterpenes, which can be produced.

Thus, the invention features an inventory of functional class II and class I diTPS from a range of plants, which are useful for accumulating high-value and bioactive diterpenes. When these diTPS are paired into specific modules consisting of new-to-nature combinations, such as using enzymes from different plant species, both the structure and the stereochemistry of the formed diterpenes can be controlled. This strategy gives access to a novel structural diversity of highly complex diterpenes, representing potentially bioactive molecules, starting materials for chemical synthesis, and intermediates for further functionalization to flavours, fragrances, pharmaceuticals and fine chemicals.

The invention thus in one aspect provides methods of producing a terpene, said methods comprising the steps of:
a) providing a host organism comprising
   I. A heterologous nucleic acid encoding a diTPS of class II,
   II. A heterologous nucleic acid encoding a diTPS of class I,
   with the proviso that said diTPS of class II and said diTPS of class I is not from the same species;
b) Incubating said host organism in the presence of geranylgeranyl pyrophosphate (GGPP) under conditions allowing growth of said host organism;
c) Optionally isolating diterpene from the host organism.

The invention further provides host organisms, comprising
I. A heterologous nucleic acid encoding a diTPS of class II;
II. A heterologous nucleic acid encoding a diTPS of class I,
with the proviso that said diTPS of class II and said diTPS of class I is not from the same species.

Said host organism may for example be any of the host organisms described herein below in the section “Host organism”.

It is preferred that the combination of diTPS of class II and diTPS of class I is not found in nature. Thus, it is preferred that the diTPS of class II and the diTPS of class I is not from the same species. Accordingly, if the diTPS of class I is from species X or highly similar to a diTPS of class I of species X, then it is preferred that the diTPS of class II does not have a sequence identity of more than 95%, such as of more than 90%, for example of more than 80%, such as of more than 70% to any diTPS of class II of species X. Similarly, if the diTPS of class II is from species X of highly similar to a diTPS of class II of species X, then it is preferred that the diTPS of class I does not have a sequence identity of more than 95%, such as of more than 90%, for example of more than 80%, such as of more than 70% to any diTPS of class I of species X. In this connection the term “highly similar” means sharing more than 95%, such as of more than 90%, for example of more than 80%, such as of more than 70% sequence identity.

The invention also provides several enzymes useful with the methods of the invention. Thus, the invention provides EpTPS7 like diTPS enzymes, such as EpTPS7 of SEQ ID NO:2 or a functional homologue thereof sharing at least 70%, such as at least 80%, such as at least 85%, such as at least 90%, such as at least 95% sequence identity therewith.

The invention also provides TwTPS7 like diTPS enzymes, such as TwTPS7 of SEQ ID NO:4 or a functional homologue thereof sharing at least 70%, such as at least 80%, such as at least 85%, such as at least 90%, such as at least 95% sequence identity therewith.

The invention also provides CfTPS1 like diTPS enzymes, such as CfTPS1 of SEQ ID NO:5 or a functional homologue thereof sharing at least 70%, such as at least 80%, such as at least 85%, such as at least 90%, such as at least 95% sequence identity therewith.

The invention also provides TwTPS21 like diTPS enzymes, such as TwTPS21 of SEQ ID NO:7 or a functional homologue thereof sharing at least 70%, such as at least 80%, such as at least 85%, such as at least 90%, such as at least 95% sequence identity therewith.

The invention also provides TwTPS14/28 like diTPS enzymes, such as TwTPS14/28 of SEQ ID NO:8 or a functional homologue thereof sharing at least 70%, such as at least 80%, such as at least 85%, such as at least 90%, such as at least 95% sequence identity therewith.

The invention also provides EpTPS8 like diTPS enzymes, such as EpTPS8 of SEQ ID NO:9 or a functional homologue thereof sharing at least 70%, such as at least 80%, such as at least 85%, such as at least 90%, such as at least 95% sequence identity therewith.

The invention also provides EpTPS23 like diTPS enzymes, such as EpTPS23 of SEQ ID NO:10 or a functional homologue thereof sharing at least 70%, such as at least 80%, such as at least 85%, such as at least 90%, such as at least 95% sequence identity therewith.

The invention also provides TwTPS2 like enzymes, such as TwTPS2 of SEQ ID NO:14 or a functional homologue thereof sharing at least 70%, such as at least 80%, such as at least 85%, such as at least 90%, such as at least 95% sequence identity therewith.

The invention also provides EpTPS1 like enzymes, such as EpTPS1 of SEQ ID NO:15 or a functional homologue thereof sharing at least 70%, such as at least 80%, such as at least 85%, such as at least 90%, such as at least 95% sequence identity therewith.

The invention also provides CfTPS14, such as CfTPS14 of SEQ ID NO:16 or a functional homologue thereof sharing at least 70%, such as at least 80%, such as at least 85%, such as at least 90%, such as at least 95% sequence identity therewith.

### DESCRIPTION OF DRAWINGS

FIG. 1 provides an example of biosynthesis pathways to diterpenes of different stereochemistry. The figure shows biosynthesis of three different isomers of manool by using diTPS enzymes from four different species: Oryza Sativa (rice), Zea maiz (maize), Coleus forskolii (medicinal plant) and Salvia sclarea (medicinal plant). The diTPS from Oryza sativa may for example be the enzyme of SEQ ID NO:1. The diTPS from Zea maiz may for example be the enzyme of SEQ ID NO:3. The diTPS from Coleus forskolii may for example be the enzyme of SEQ ID NO:5. The diTPS from Salvia sclarea may for example be the enzyme of SEQ ID NO:11.

FIGS. 2A and 2B shows “Combinatorial wheels” showing examples of compounds, which can be made by combining different diTPS enzymes. The universal precursor, GGPP is shown in the middle. The next ring shows various examples of diTPS class II enzymes. The next ring shows various examples of diTPS class I enzymes. The outer ring shows the diterpenes produced by the indicated combinations of diTPS class II and diTPS class I enzymes. Each diterpene has been assigned a compound number used to identify said diterpene herein. The sequences of all of diTPS class II and diTPS class I enzymes are provided herein in the sequence listing and MS spectras of all the diterpene compounds are given in FIG. 6. Table 1 also provides a list of the diterpenes.

FIGS. 3A and 3B show the reactions catalysed by various class II diTPS enzymes as well as the diterpene pyrophosphate intermediates generated by the reactions.

FIG. 4 shows an alignment of the amino acid sequences of selected diTPS enzymes of class I.

FIG. 5 shows an alignment of the amino acid sequences of selected diTPS enzymes of class II.

FIG. 6 shows MS spectras of hexane extracts from N. benthamiana expressing the different diTPS genes. MS spectras of all 47 diterpenes produced as described in Example 1 are shown, with the compound number indicated in the upper left corner of each spectrum. For some compounds also reference spectra are shown.

### DETAILED DESCRIPTION OF THE INVENTION

Method for Producing Diterpenes

The present invention relates to a biosynthetic method for producing diterpenes. The methods typically involves the steps of
a) Contacting GGPP with a diTPS of class II, which may be any of diTPS of class II described herein in any of the sections “diTPS of class II”, “syn-CPP type diTPS”, “ent-CPP type diTPS”, “(+)-CPP type diTPS”, “LPP type diTPS”, and “LPP like type diTPS”, thereby producing a diterpene pyrophosphate intermediate;
b) Contacting said diterpene pyrophosphate intermediate with a diTPS of class I, which may be any of diTPS of class I described herein in any of the sections “diTPS of class I”, “EpTPS8”, “EpTPS23”, “SsSCS”, “CfTPS3”, “CfTPS4”, “MvTPS5”, “TwTPS2”, “EpTPS1”, and “CfTPS14” thereby producing a diterpene.

It is generally preferred that the diTPS of class I and the diTPS of class II are not from the same species. Furthermore, it is preferred that when said diTPS of class II is SsLPPS then said diTPS of class I is preferably not CfTPS3, CfTPS4 or EpTPS8 and when said diTPS of class I is EpTPS8, then the diTPS of class II is preferably not CfTPS2 or SsLPPS. In particular, when said diTPS of class II is SsLPPS or any of the functional homologues of SsLPPS described in the section “LPP type diTPS”, then said diTPS of class I is preferably not CfTPS3 or any of the functional homologues thereof described in the section “CfTPS3”, is also preferably not CfTPS4 or any of the functional homologues thereof described in the section “CfTPS4”, and is also preferably not EpTPS8 or any of the functional homologues thereof described in the section EpTPS8. It is also preferred that when said diTPS of class I is EpTPS8 or any of the functional homologues thereof described in the section “EpTPS8”, then the diTPS of class II is preferably not CfTPS2 or any of the functional homologues thereof described in the section “LPP type diTPS” or SsLPPS or any of the functional homologues thereof described in the section “LPP type diTPS”.

The method may be performed in vitro or in vivo.

The diterpene pyrophosphate intermediate and the diterpene may for example be any of the compounds described herein below in the sections “Diterpene pyrophosphate intermediates” and “Diterpenes”.

When the methods are performed in vitro, the above-mentioned steps a) and b) may be performed individually in the indicated sequence, or they may be performed simultaneously. When both steps are performed simultaneously GGPP and the diTPS of class II and the diTPS of class I may all be incubated in the same container under conditions allowing activity of both the diTPS of class II and the diTPS of class I. When the steps are performed sequentially, the step a) may be performed first in one container, whereafter the diTPS of class I may be added to the container. It is also possible that the diterpene pyrophosphate intermediate may be purified or partly purified after step a) and then it may be contacted with the diTPS of class I e.g. in another container.

When the methods are performed in vitro they may contain the steps of providing a host organism comprising
a. A heterologous nucleic acid encoding a diTPS of class II, which may be any of diTPS of class II described herein in any of the sections “diTPS of class II”, “syn-CPP type diTPS”, “ent-CPP type diTPS”, “(+)-CPP type diTPS”, “LPP type diTPS”, and “LPP like type diTPS” and/or
b. A heterologous nucleic acid encoding a diTPS of class I, which may be any of diTPS of class I described herein in any of the sections “diTPS of class I”, “EpTPS8”, “EpTPS23”, “SsSCS”, “CfTPS3”, “CfTPS4”, “MvTPS5”, “TwTPS2”, “EpTPS1”, and “CfTPS14”;
   b) preparing an extract of said host organism;
   c) providing GGPP
   d) incubating said extract with GGPP
      thereby producing a diterpene.

When the methods are performed in vitro they may also contain the steps of
a) providing a host organism comprising a heterologous nucleic acid encoding a diTPS of class II, which may be any of diTPS of class II described herein in any of the sections “diTPS of class II”, “syn-CPP type diTPS”, “ent-CPP type diTPS”, “(+)-CPP type diTPS”. “LPP type diTPS”, and “LPP like type diTPS”; and
b) Preparing an extract of said host organism
c) Providing another host organism comprising a heterologous nucleic acid encoding a diTPS of class I, which may be any of diTPS of class I described herein in any of the sections “diTPS of class I”, “EpTPS8”, “EpTPS23”, “SsSCS”, “CfTPS3”, “CfTPS4”, “MvTPS5”, “TwTPS2”, “EpTPS1”, and “CfTPS14”;
d) preparing an extract of the host organism of c); and
e) providing GGPP
f) incubating the extract of step b) and the extract of d) with GGPP OR incubating the extract of b) with GGPP followed by incubating the product with the extract of d)
   thereby producing a diterpene.

In a preferred embodiment of the invention the methods are performed in vivo. The term “in vivo” as used herein refers that the method is performed within a host organism, which for example may be any of the host organisms described herein below in the section “Host organism”. In embodiments of the invention wherein the methods are performed in vivo, it is preferred that steps a) and b) are performed simultaneously. Thus, the methods may comprise the steps of
I. Providing a host organism comprising
   a. A heterologous nucleic acid encoding a diTPS of class II, which may be any of diTPS of class II described herein in any of the sections “diTPS of class II”, “syn-CPP type diTPS”, “ent-CPP type diTPS” “(+)-CPP type diTPS”, “LPP type diTPS”, and “LPP like type diTPS”,
   b. A heterologous nucleic acid encoding a diTPS of class I, which may be any of diTPS of class I described herein in any of the sections “diTPS of class I”, “EpTPS8”, “EpTPS23”, “SsSCS”, “CfTPS3”, “CfTPS4”, “MvTPS5”, “TwTPS2”, “EpTPS1”, and “CfTPS14”
II. Incubating said host organism in the presence of GGPP under conditions allowing growth of said host organism
III. Optionally isolating the diterpene from the host organism.

The in vivo methods may also be performed in a manner, wherein steps a) and b) are performed sequentially. Thus, the methods may comprise the steps of
I. Providing a host organism comprising
   a. A heterologous nucleic acid encoding a diTPS of class II, which may be any of diTPS of class II described herein in any of the sections “diTPS of class II”, “syn-CPP type diTPS”, “ent-CPP type diTPS”, “(+)-CPP type diTPS”, “LPP type diTPS”, and “LPP like type diTPS”,
II. Incubating said host organism in the presence of GGPP under conditions allowing growth of said host organism, thereby producing a diterpene pyrophosphate intermediate
III. Providing a host organism comprising
   a. A heterologous nucleic acid encoding a diTPS of class I, which may be any of diTPS of class I described herein in any of the sections “diTPS of class I”, “EpTPS8”, “EpTPS23”, “SsSCS”, “CfTPS3”, “CfTPS4”, “MvTPS5”, “TwTPS2”, “EpTPS1”, and “CfTPS14”
IV. Incubating said host organism in the presence of the diterpene pyrophosphate intermediate produced in step II. under conditions allowing growth of said host organism, thereby producing a diterpene
V. Optionally isolating the diterpene.

In preferred embodiments of the invention the host organism is capable of producing GGPP. Thus step II. may simply be performed by cultivating said host organism. Many host organisms produce GGPP endogenously. Thus, the host organism may be a host organism, which endogenously produce GGPP. Such host organisms for example include plants and yeast. Even if the host organism produce GGPP endogenously, the host organism may be recombinantly modulated to upregulate production of GGPP.

It is also comprised within the invention that GGPP is introduced to the host organism. If the host organism is a microorganism, then GGPP may be added to the cultivation medium of said microorganism. If the host organism is a plant, then GGPP may be added to the growing soil of the plant or it may be introduced into the plant by infiltration. Thus, if the heterologous nucleic(s) are introduced into the plant by infiltration, then GGPP may be co-infiltrated together with the heterologous nucleic acid(s).

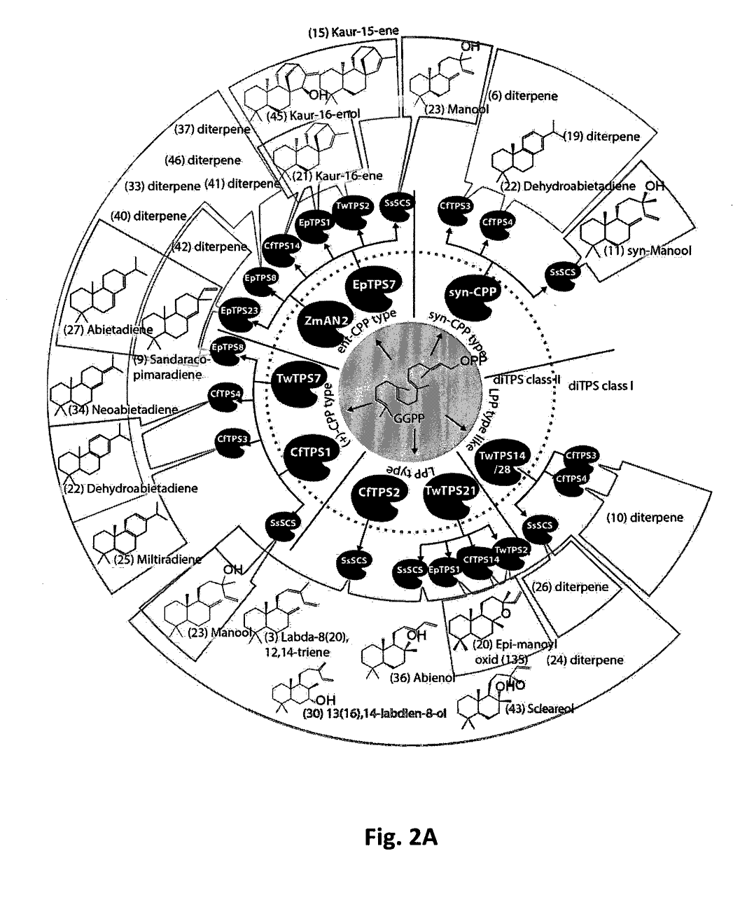

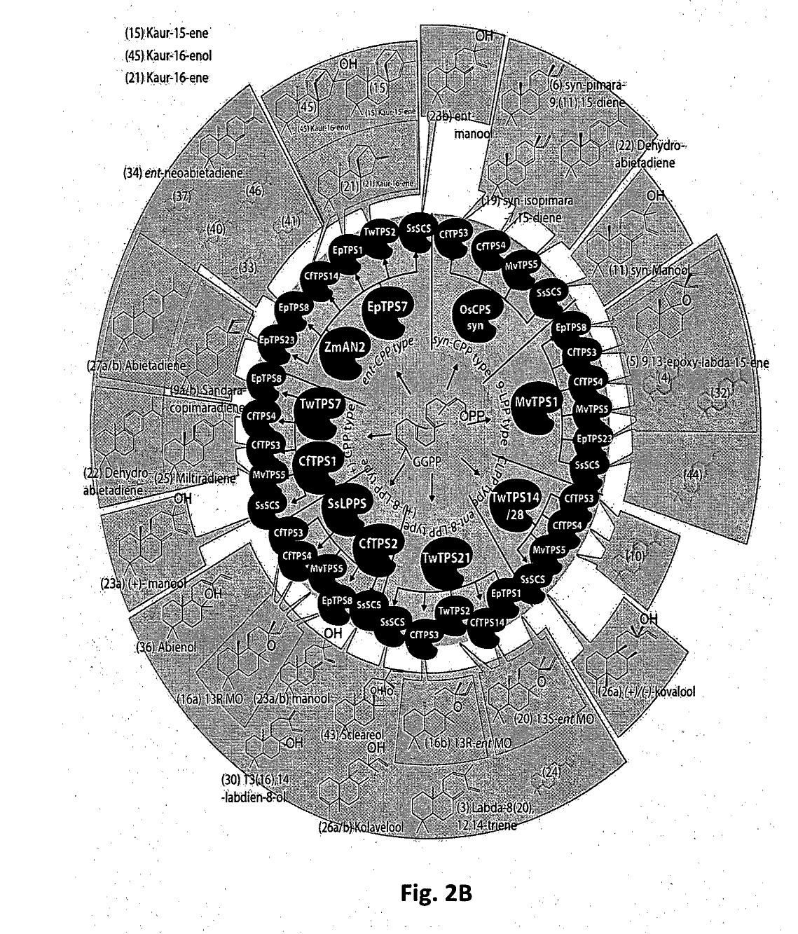

In order to produce a specific diterpene according to the present invention, a useful combination of a diTPS of class II and a diTPS of class I must be employed. Examples of specific combinations of a diTPS of class II and a diTPS of class I, which leads to production of specific diterpenes are shown in FIG. 2. Other combinations of diTPS of class II and diTPS of class I may be used. In general, the diTPS of class II is selected so that it produces a diterpene pyrophosphate intermediate containing a decalin core having the desired stereochemistry at the 9 and 10 substitutions. Useful diTPS of class II are described below and also specific diTPS of class II catalysing formation of diterpene pyrophosphate intermediates with a specific stereochemistry are described. The diTPS of class I is selected so that is catalyses the conversion of the diterpene pyrophosphate intermediate to the desired diterpene. Useful diTPS of class I are described below. Also specific reactions catalysed by various diTPS of class I are described, enabling the skilled person to select a useful diTPS of class I for production of a desired diterpene. Once a useful diTPS of class II and diTPS of class I have been selected, nucleic acids encoding same may be expressed in the host organism allowing production of the diterpene in the host organism. Putative useful combinations of a diTPS of class II and a diTPS of class I for production of a given diterpene may be tested by expressing said diTPS of class II and said diTPS of class I in a host organism followed by testing for production of the diterpene, e.g. by GC-MS analysis and/or NMR analysis. Putative useful combinations of a diTPS of class II and a diTPS of class I for production of a given diterpene may in particular be tested as described in Example 1 herein below. Methods for expression of enzymes in host organisms are well known to skilled person, and may for example include the methods described herein below in the section “Heterologous nucleic acids”.

The term GGPP as used herein refers to geranylgeranyl diphosphate and is a compound of the following structure:

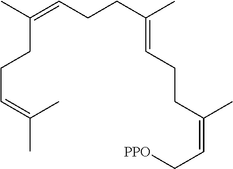

wherein PPO— is diphospjhate. PPO— and —OPP may be used interchangeably herein.

diTPS of Class II

The methods of the invention comprise step a), which involves use of a diTPS of class II. The invention also features host organisms comprising a heterologous nucleic acid encoding a diTPS of class II. The invention also relates to certain diTPS of class II per se.

Said diTPS of class II is an enzyme capable of catalysing protonation-initiated cationic cycloisomerization of GGPP to form a diterpene pyrophosphate intermediate. The class II diTPS reaction, may be terminated either by deprotonation or by water capture of the diphosphate carbocation.

In particular the diTPS of class II may be an enzyme capable of catalysing the reaction I:

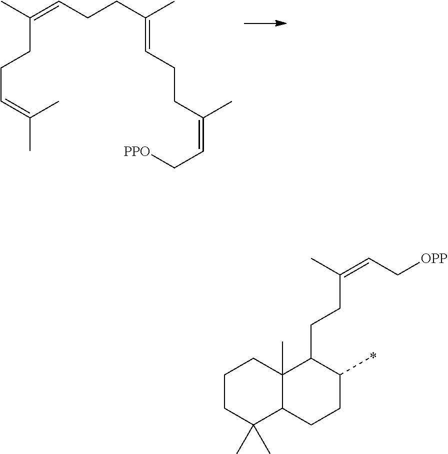

wherein PPO— is diphosphate and the [symbol] indicates either a double bond or two single bonds, wherein one is substituted with —OH and the other with —CH3.

Thus, [symbol] may be [symbol] or [symbol] .

When no stereochemistry is indicated, the bond may be in any conformation. By selecting appropriate diTPS of class II the stereochemistry of the diterpene produced may be controlled. Accordingly. by following the description of the present invention, the skilled person may be able to design the production of a given diterpene by selecting appropriate diTPS enzymes of class II and class I as described herein.

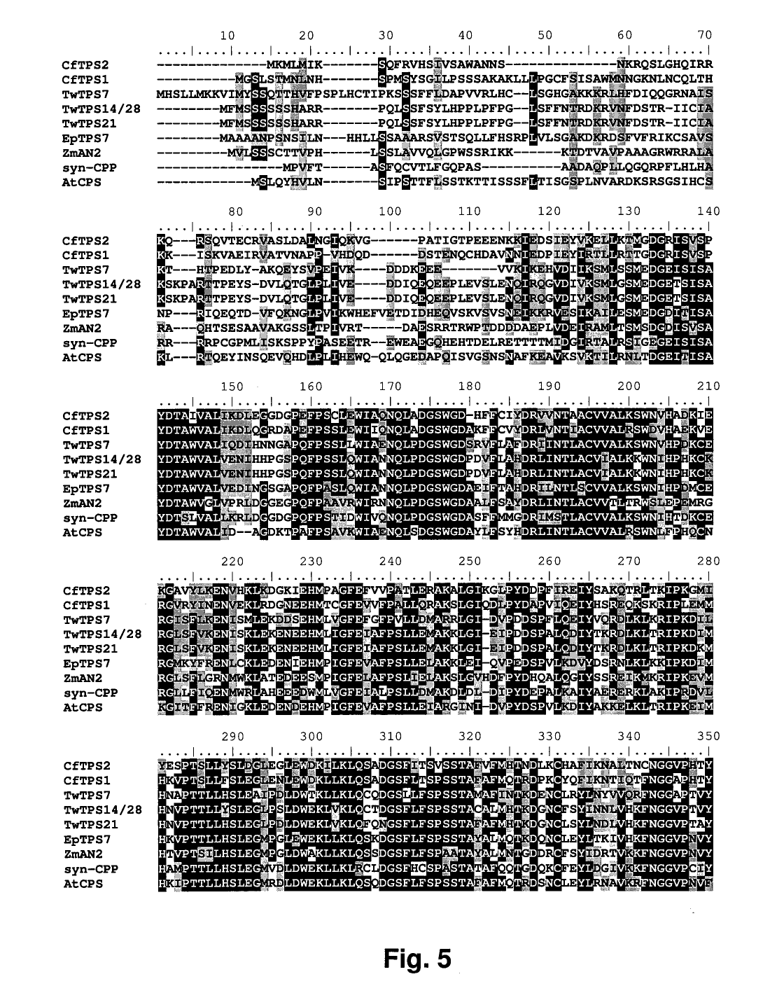

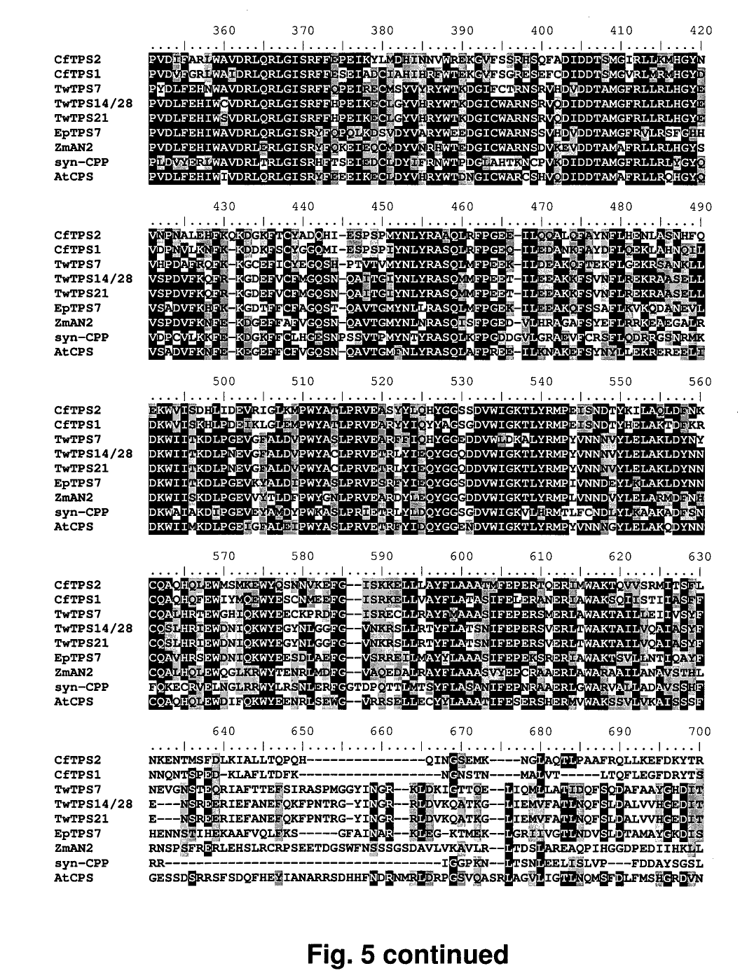

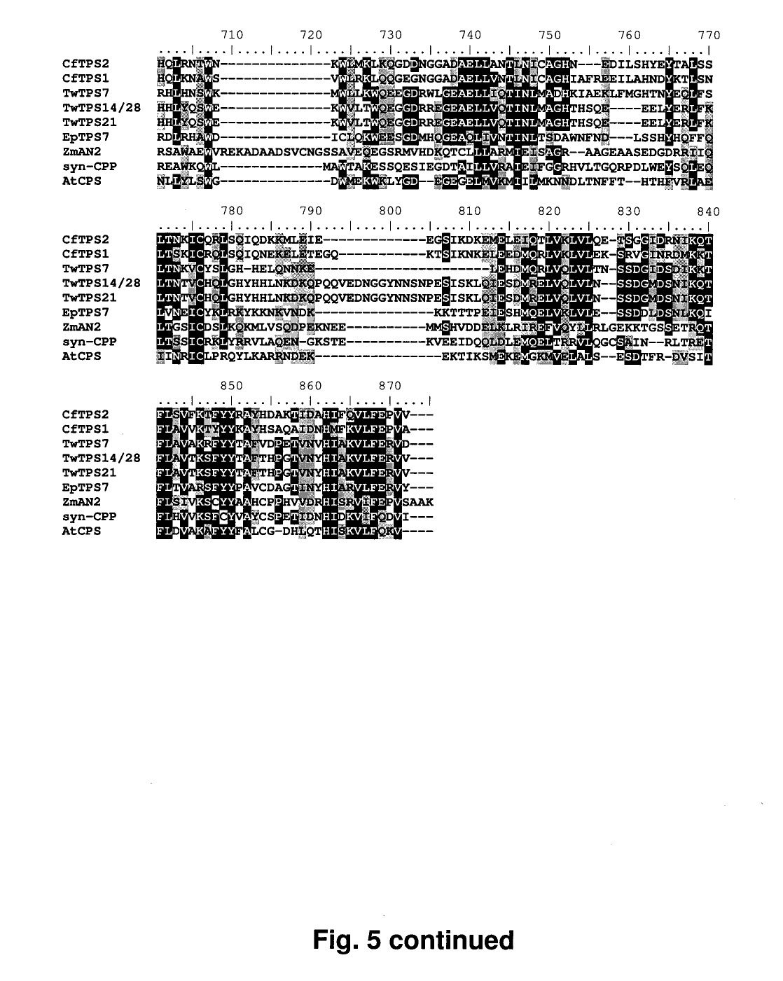

The diTPS of class II is generally a polypeptide sharing at least some sequence similarity to at least one of SEQ ID NO:1, SEQ ID NO:2, SEQ ID NO:3, SEQ ID NO:4, SEQ ID NO:5, SEQ ID NO:6, SEQ ID NO:7 or SEQ ID NO:8. In particular, it is preferred that the diTPS of class II shares at least 30%, preferably at least 40% sequence identity with at least one of SEQ ID NO:1. SEQ ID NO:2, SEQ ID NO:3, SEQ ID NO:4, SEQ ID NO:5, SEQ ID NO:6, SEQ ID NO:7 and SEQ ID NO:8. In particular, it is preferred that the diTPS of class II shares at least 30%, such as at least 35% sequence identity to the sequence of SsLPPS (SEQ ID NO:6) or to the sequence of AtCPS (see FIG. 5). Furthermore, it is preferred that the diTPS of class II in addition to above mentioned sequence identity also contains the following motif of four amino acids:

D/E-X-D-D,

wherein X may be any amino acid, such as any naturally occurring amino acids. In particular, X may be an amino acid with a hydrophobic side chain, and thus X may for example be selected from the group consisting of A, I, L, M, F, W, Y and V. Even more preferably X is an amino acid with a small hydrophobic side chain, and thus X may be selected from the group consisting of A, I, L and V.

In one embodiment of the invention said motif of four amino acids is:

D/E-I/V-D-D

D/E indicates that said amino acid may be D or E and I/V indicates that said amino acid may be I or V.

Amino acids are herein named using the IUPAC nomenclature for amino acids.

In particular, it is preferred that the diTPS of class II contains above described motif in a position corresponding to position aa 372 to 375 of SsLPPS of SEQ ID NO:6. A position corresponding to position aa 372 to 375 of SsLPPS of SEQ ID NO:6 is identified by aligning the sequence of a diTPS of class II of interest to SEQ ID NO:6 and optionally to additional sequences of diTPS of class II as e.g. shown in FIG. 5 and identifying the amino acids of said diTPS of class II aligning with aa 372 to 375 of SsLPPS of SEQ ID NO:6.

It is furthermore preferred that in addition to sharing above mentioned sequence identity and containing said motif, then as many as possible of the amino acids marked with a black box in FIG. 5 are retained. Thus, when aligned to the sequence of ScLPPS (SEQ ID NO:6), then preferably the diTPS of class II also contains at least 80%, more preferably at least 90%, for example at least 95%, such as all of the amino acids marked by a black box in FIG. 5. Alternatively, when aligned to the sequence of sequence of AtCPS (see FIG. 5), then preferably the diTPS of class II also contains at least 80%, more preferably at least 90%, for example at least 95%, such as all of the amino acids marked by a black box in FIG. 5.

Thus, the diTPS of class II may for example be selected from the group consisting of diTPS of class II of the following types:
i. syn-CPP type, such as any of the enzymes described herein below in the section “syn-CPP type diTPS”
ii. ent-CPP type, such as any of the enzymes described herein below in the section “ent-CPP type diTPS”
iii. (+)-CPP type, such as any of the enzymes described herein below in the section “(+)-CPP type diTPS”
iv. LPP type, such as any of the such as any of the enzymes described herein below in the section “LPP type diTPS”
v. LPP like type, such as any of the enzymes described herein below in the section “LPP like type diTPS”

Certain diTPS enzymes are bifunctional in the sense that they may be classified as both class II and class I diTPS enzymes. Such bifunctional diTPS enzymes in general contain both the four amino acids motif: D/E-X-D-D, described herein above, as well as the five amino acid motif: D-D-X—X-D/E, described herein below. It is preferred that the diTPS of class II is not a bifunctional enzyme of both class II and class I. It is also preferred that the diTPS of class I is not a bifunctional enzyme of both class II and class I.

Syn-CPP Type diTPS

The methods of the invention comprise step a), which involves use of a diTPS of class II. The invention also features host organisms comprising a heterologous nucleic acid encoding a diTPS of class II. The invention also relates to certain diTPS of class II per se. In one embodiment said diTPS of class II is a syn-CPP type diTPS. Such diTPS of class II are in particular useful in embodiments of the inventions, wherein the diterpene to be produced contains a 9S,10R decalin core.

As used herein the term “syn-CPP type diTPS” refers to any enzyme capable of catalysing the reaction II:

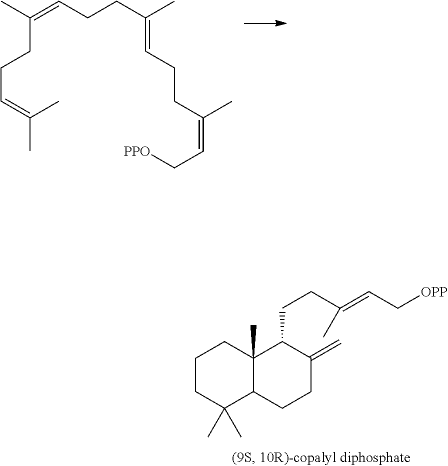

wherein PPO— refers to diphosphate.

In one embodiment the syn-CPP type diTPS may be syn-copalyl pyrophosphate synthase (syn-CPP), such as syn-CPP from Oryza sativa. In particular, said syn-CPP type diTPS may be a polypeptide of SEQ ID NO:1 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. The sequence identity is preferably calculated as described herein below in the section “Sequence identity”. A functional homologue of a syn-CPP is a polypeptide, which is also capable of catalysing reaction II described above.

Ent-CPP Type

The methods of the invention comprise step a), which involves use of a diTPS of class II. The invention also features host organisms comprising a heterologous nucleic acid encoding a diTPS of class II. The invention also relates to certain diTPS of class II per se. In one embodiment said diTPS of class II is an ent-CPP type diTPS. Such diTPS of class II are in particular useful in embodiments of the inventions, wherein the diterpene to be produced contains a 9R,10R decalin core.

As used herein the term “ent-CPP type diTPS” refers to any enzyme capable of catalysing the reaction III:

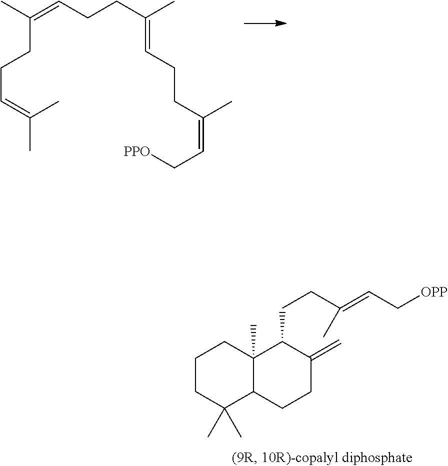

wherein PPO— refers to diphosphate.

In one embodiment the ent-CPP type diTPS may be EpTPS7. In particular, said ent-CPP type diTPS may be a polypeptide of SEQ ID NO:2 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In another embodiment the ent-CPP type diTPS may be ZmAN2. In particular, said ent-CPP type diTPS may be a polypeptide of SEQ ID NO:3 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of an ent-CPP is a polypeptide, which is also capable of catalysing reaction III described above.

(+)-CPP Type diTPS

The methods of the invention comprise step a), which involves use of a diTPS of class II. The invention also features host organisms comprising a heterologous nucleic acid encoding a diTPS of class II. The invention also relates to certain diTPS of class II per se. In one embodiment said diTPS of class II is a (+)-CPP type diTPS. Such diTPS of class II are in particular useful in embodiments of the inventions, wherein the diterpene to be produced contains a 9S,10S decalin core.

As used herein the term “(+)-CPP type diTPS” refers to any enzyme capable of catalysing the reaction IV:

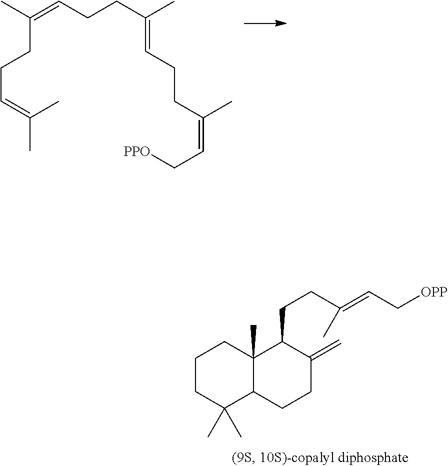

wherein PPO— refers to diphosphate.

In one embodiment the (+)-CPP type diTPS may be TwTPS7. In particular, said (+)-CPP type diTPS may be a polypeptide of SEQ ID NO:4 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In another embodiment the (+)-CPP type diTPS may be CfTPS1. In particular, said (+)-CPP type diTPS may be a polypeptide of SEQ ID NO:5 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of a (+)-CPP is a polypeptide, which is also capable of catalysing reaction IV described above.

LPP Type diTPS

The methods of the invention comprise step a), which involves use of a diTPS of class II. The invention also features host organisms comprising a heterologous nucleic acid encoding a diTPS of class II. The invention also relates to certain diTPS of class II per se. In one embodiment said diTPS of class II is a LPP type diTPS. Such diTPS of class II are in particular useful in embodiments of the inventions, wherein the diterpene to be produced contains a 8-hydroxy-decalin core. However, LPP type diTPS may also be useful in other embodiments of the invention.

As used herein the term “LPP type diTPS” refers to any enzyme capable of catalysing the reaction V:

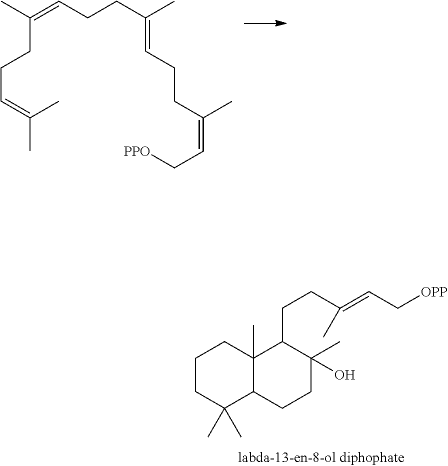

wherein PPO— refers to diphosphate.

In one embodiment the LPP type diTPS may be labda-13-en-8-ol pyrophosphate synthase, such as SsLPPS. In particular, said LPP type diTPS may be a polypeptide of SEQ ID NO:6 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. In embodiments of the invention, wherein the diTPS of class II is SsLPPS or a functional homologue thereof sharing above mentioned sequence identity, then it is preferred that the diTPS of class I is not SsSCS [SEQ ID NO:11], CfTPS3 [SEQ ID NO:12], CfTPS4 [SEQ ID NO:13] or EpTPS8 [SEQ ID NO:9] or a functional homologue of any of the aforementioned sharing at least 70% sequence identity therewith. Thus, in embodiments of the invention, wherein the diTPS of class II is SsLPPS, then it is preferred that the diTPS of class I is not SsSCS, CfTPS3, CfTPS4 or EpTPS8. It is also preferred that if the diTPS of class II is SsCPSL, then it is preferred that the diTPS of class I is not SsKSL1 or SsKSL2.

In another embodiment the LPP type diTPS may be TwTPS21. In particular, said LPP type diTPS may be a polypeptide of SEQ ID NO:7 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In another embodiment the LPP type diTPS may be CfTPS2. In particular, said LPP type diTPS may be a polypeptide of SEQ ID NO:17 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. In embodiments of the invention, wherein the diTPS of class II is CfTPS2 or a functional homologue thereof sharing above mentioned sequence identity, then it is preferred that the diTPS of class I is not CfTPS3 [SEQ ID NO:12] or CfTPS4 [SEQ ID NO:13] or EpTPS8 [SEQ ID NO:9] or a functional homologue of any of the aforementioned sharing at least 70% sequence identity therewith. Thus, in embodiments of the invention, wherein the diTPS of class II is CfTPS2, then it is preferred that the diTPS of class I is not CfTPS3 or CfTPS4 or EpTPS8.

The sequence identity is preferably calculated as described herein below in the section “Sequence identity”. A functional homologue of a LPP is a polypeptide, which is also capable of catalysing reaction V described above.

The LLP type diTPS may be an (+)-LPP type diTPS or an ent-LPP type diTPS. Thus, in one embodiment of the invention, the diTPS of class II is an (+)-LPP type diTPS.

As used herein the term “(+)-LPP type diTPS” refers to any enzyme capable of catalysing the reaction XXXIII:

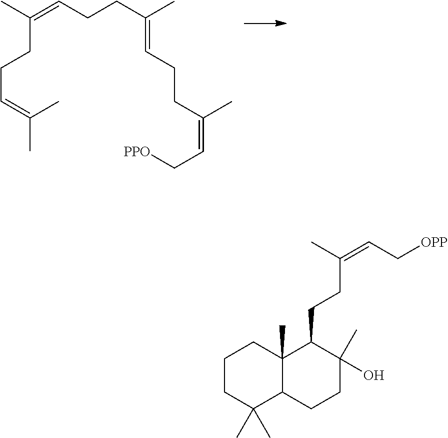

wherein —OPP refers to diphosphate.

In one embodiment the (+)-LPP type diTPS may be labda-13-en-8-ol pyrophosphate synthase, such as SsLPPS. In particular, said (+)-LPP type diTPS may be a polypeptide of SEQ ID NO:6 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. In embodiments of the invention, wherein the diTPS of class II is SsLPPS or a functional homologue thereof sharing above mentioned sequence identity, then it is preferred that the diTPS of class I is not SsSCS [SEQ ID NO:11], CfTPS3 [SEQ ID NO:12], CfTPS4 [SEQ ID NO:13] or EpTPS8 [SEQ ID NO:9] or a functional homologue of any of the aforementioned sharing at least 70% sequence identity therewith. Thus, in embodiments of the invention, wherein the diTPS of class II is SsLPPS, then it is preferred that the diTPS of class I is not SsSCS, CfTPS3, CfTPS4 or EpTPS8

In one embodiment of the invention, the diTPS of class IIis an ent-LPP type diTPS.

As used herein the term “ent-LPP type diTPS” refers to any enzyme capable of catalysing the reaction XXXIV:

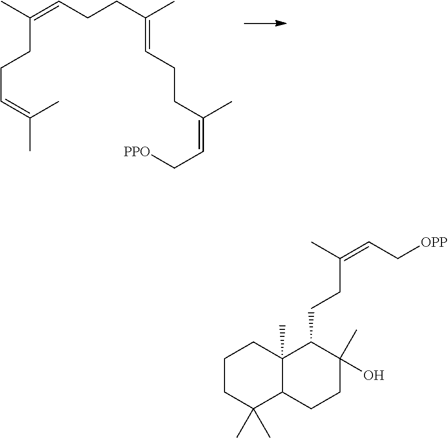

wherein —OPP refers to diphosphate.

In one embodiment the ent-LPP type diTPS may be TwTPS21. In particular, said net-LPP type diTPS may be a polypeptide of SEQ ID NO:7 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

LPP Like Type diTPS

The methods of the invention comprise step a), which involves use of a diTPS of class II. The invention also features host organisms comprising a heterologous nucleic acid encoding a diTPS of class II. The invention also relates to certain diTPS of class II per se. In one embodiment said diTPS of class II is a LPP like type diTPS.

In one embodiment the LPP like type diTPS may be TwTPS14/28. In particular, said LPP like type diTPS may be a polypeptide of SEQ ID NO:8 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The LPP like type diTPS may in one embodiment be a CLPP type diTPS.

As used herein the term “CLPP type diTPS” refers to any enzyme capable of catalysing the reaction XXXV:

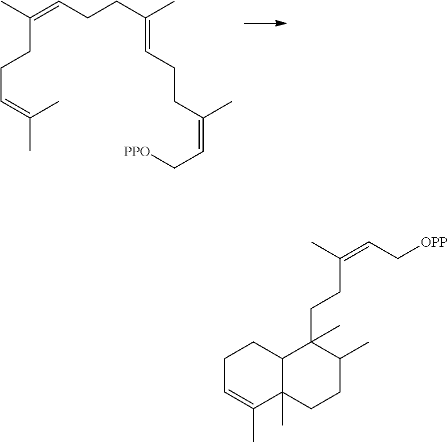

wherein PPO— refers to diphosphate.

The CLPP type diTPS may for example be TwTPS14/28. In particular, said CLPP type diTPS may be a polypeptide of SEQ ID NO:8 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. A functional homologue of TwTPS14/28 may in particular be a polypeptide have aforementioned sequence identity with TwTPS14/28 and which also is capable of catalysing reaction XXXV.

The LPP like type diTPS may in one embodiment be a 9-LPP type diTPS.

As used herein the term “9-LPP type diTPS” refers to any enzyme capable of catalysing the reaction XXXVI:

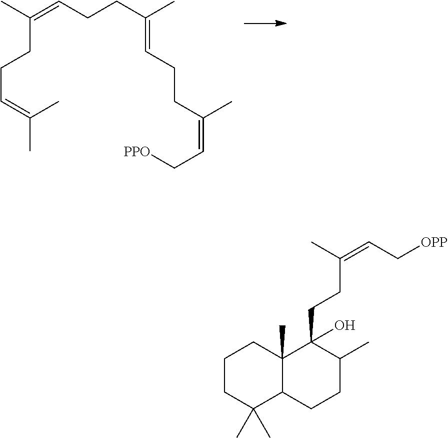

wherein PPO— refers to diphosphate.

The 9-LPP type diTPS may for example be MvTPS1. In particular, said 9-LPP type diTPS may be a polypeptide of SEQ ID NO:28 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. A functional homologue of MvTPS1 may in particular be a polypeptide have aforementioned sequence identity with MvTPS1 and which also is capable of catalysing reaction XXXVI.

The sequence identity is preferably calculated as described herein below in the section “Sequence identity”.

diTPS of Class I

The methods of the invention comprise step b), which involves use of a diTPS of class I. The invention also features host organisms comprising a heterologous nucleic acid encoding a diTPS of class I. The invention also relates to certain diTPS of class I per se.

Said diTPS of class I is an enzyme capable of catalyzing cleavage of the diphosphate group of the diterpene pyrophosphate intermediate and additionally preferably also is capable of catalysing cyclization and/or rearrangement reactions on the resulting carbocation. As with the class II diTPSs, deprotonation or water capture may terminate the class I diTPS reaction leading to hydroxylation of the diterpene pyrophosphate intermediate.

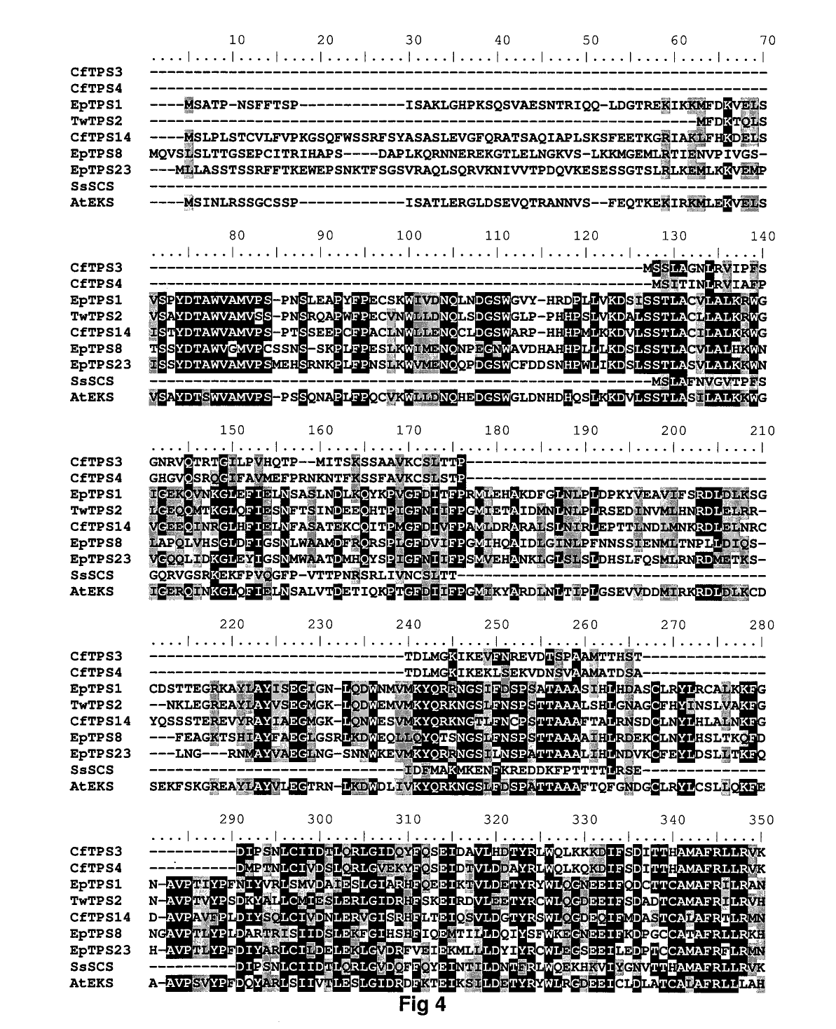

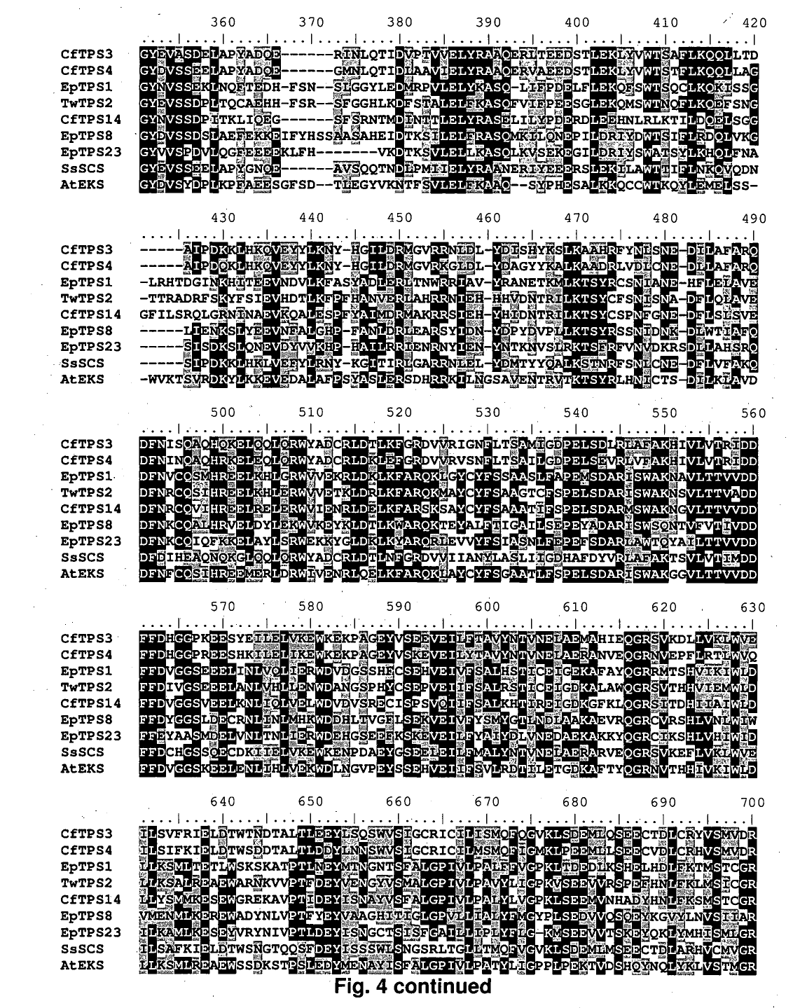

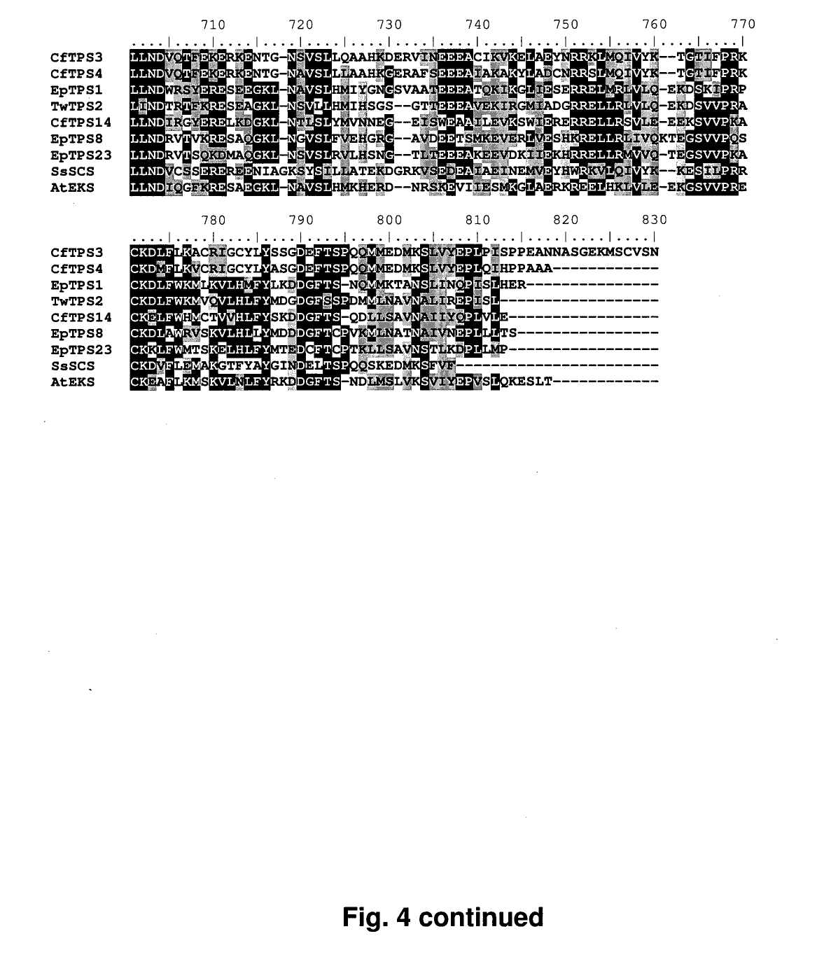

The diTPS of class I is generally a polypeptide sharing at least some sequence similarity to at least one of SEQ ID NO:9, SEQ ID NO:10, SEQ ID NO:11, SEQ ID NO:14, SEQ ID NO:15, SEQ ID NO:16 or SEQ ID NO:17. In particular, it is preferred that the diTPS of class I shares at least 30%, preferably at least 40%, more preferably at least 45% sequence identity with at least one of SEQ ID NO:9, SEQ ID NO:10, SEQ ID NO:11, SEQ ID NO:14, SEQ ID NO:15, SEQ ID NO:16 and SEQ ID NO:17. In particular, it is preferred that the diTPS of class I shares at least 30%, such as at least 35% sequence identity to the sequence of ScSCS (SEQ ID NO:11) or to the sequence of AtEKS (see FIG. 4). Furthermore, it is preferred that the diTPS of class I in addition to above mentioned sequence identity also contains the following motif of five amino acids:

D-D-X—X-D/E,

wherein X may be any amino acid, such as any naturally occurring amino acids. In particular, X may be an amino acid with a hydrophobic side chain, and thus X may for example be selected from the group consisting of A, I, L, M, F, W, Y and V. Even more preferably X is an amino acid with a small hydrophobic side chain, and thus X may be selected from the group consisting of A, I, L and V.

In one embodiment of the invention said motif of five amino acids is:

D-D-F—F-D/E

D/E indicates that said amino acid may be D or E.

In particular, it is preferred that the diTPS of class I contains said motif in a position corresponding to position aa 329-333 of SsSCS of SEQ ID NO:11. A position corresponding to position aa 329-333 of SsSCS of SEQ ID NO:11 is identified by aligning the sequence of a diTPS of class I of interest to SEQ ID NO:11 and optionally to additional sequences of diTPS of class I as e.g. shown in FIG. 4, and identifying the amino acids of said diTPS of class I aligned with aa 329-333 of SsSCS of SEQ ID NO:11.

It is furthermore preferred that in addition to sharing above mentioned sequence identity and containing said motif, then as many as possible of the amino acids marked with a black box in FIG. 4 are retained. Thus, when aligned to the sequence of ScSCS (SEQ ID NO:11), then preferably the diTPS of class I also contains at least 80%, more preferably at least 90%, for example at least 95%, such as all of the amino acids marked by a black box in FIG. 4. Alternatively, when aligned to the sequence of sequence of AtEKS (see FIG. 4), then preferably the diTPS of class I also contains at least 80%, more preferably at least 90%, for example at least 95%, such as all of the amino acids marked by a black box in FIG. 4.

Thus, the diTPS of class I may for example be selected from the group consisting of diTPS of class I of the following types:
i. EpTPS8 like diTPS, such as any of the enzymes described herein below in the section “EpTPS8”
ii. EpTPS23 like diTPS, such as any of the enzymes described herein below in the section “EpTPS23”
iii. SsSCS like diTPS, such as any of the enzymes described herein below in the section “SsSCS”
iv. CfTPS3 like diTPS, such as any of the enzymes described herein below in the section “CfTPS3”
v. CfTPS4 like diTPS, such as any of the enzymes described herein below in the section “CfTPS4”
vi. TwTPS2 like diTPS, such as any of the enzymes described herein below in the section “TwTPS2”
vii. EpTPS1 like diTPS, such as any of the enzymes described herein below in the section “TwTPS1”
viii. CfTPS14 like diTPS, such as any of the enzymes described herein below in the section “CfTPS14”

The diTPS of class I may in one embodiment also be MvTPS5 like diTPS, such as any of the enzymes described herein below in the section “MvTPS5”.

EpTPS8

The invention involves use of a diTPS of class I. In one embodiment said diTPS of class I may be an EpTPS8 like diTPS. In embodiments of the invention, wherein the diTPS of class I is a EpTPS8 like diTPS, then it is preferred that the diTPS of class II is not CfTPS2[SEQ ID NO:17], or SsLPPS [SEQ ID NO:6] or a functional homologue of any of the aforementioned sharing at least 70% sequence identity therewith. Thus, in embodiments of the invention, wherein the diTPS of class I is EpTPS8, then it is preferred that the diTPS of class II is not CfTPS2 or SsLPPS.

In particular, said diTPS of class I may be an EpTPS8 like diTPS in embodiments of the invention, wherein the diterpene to be produced contains a tricyclic ring structure. For example said diTPS of class I may be and EpTPS8 like diTPS in embodiments of the invention, wherein the diterpene to be produced contains a core of any of the formulas I, II, III, VI, XXII, XXIII, XXIV or XXV:

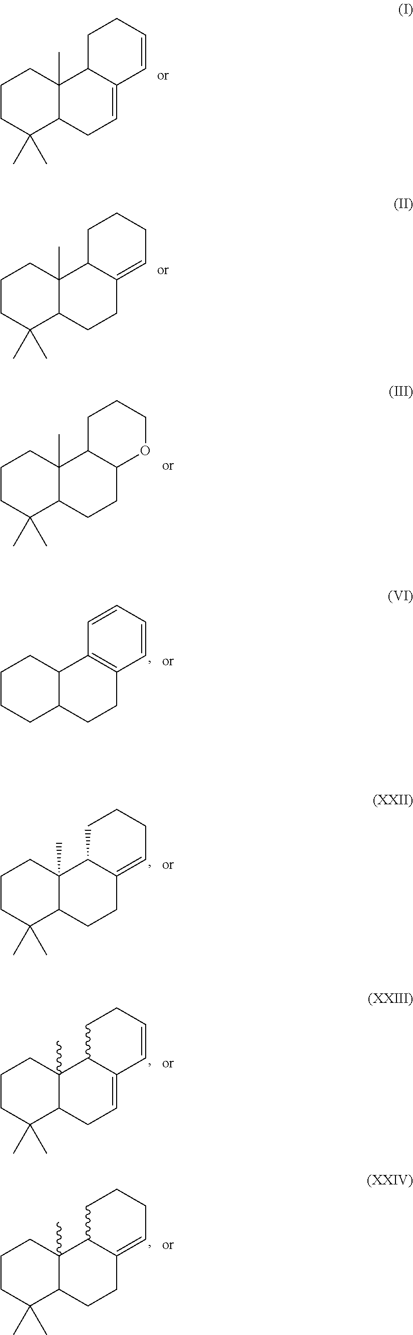

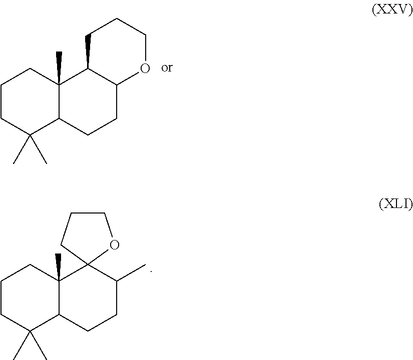

The waved line “ [symbol] ” as used herein indicates a bond of undefined stereochemistry, i.e. the bond may be either a “ [symbol] ” or “ [symbol] ”.

Dependent on the structure of the diterpene pyrophosphate intermediate then the diterpene containing a core of formula I or II may have different stereochemistry. In general the stereochemistry of the decalin core present in the diterpene pyrophosphate intermediate is maintained after the reaction catalysed by a EpTPS8 like diTPS.

The EpTPS8 like diTPS may be any enzyme capable of catalysing the reaction VII:

Diterpene pyrophosphate intermediate containing a decalin core structure→Diterpene containing a core structure of formula I or formula II or formula III or formula VI.

In particular EpTPS8 like diTPS may be an enzyme catalysing the reaction VIII:

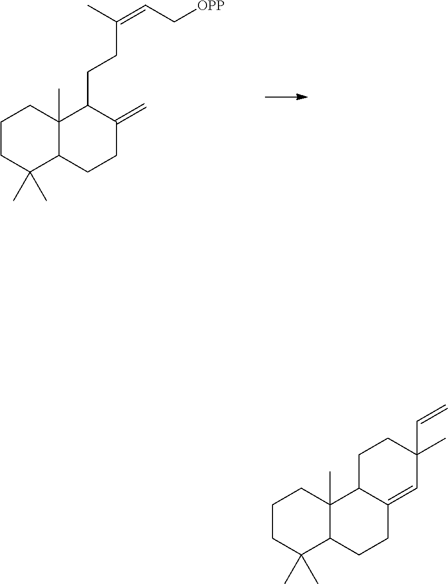

wherein —OPP indicates diphosphate. During reaction VIII the produced diterpene will in general maintain the stereochemistry around the decalin core found in the starting diterpene pyrophosphate intermediate.

The EpTPS8 like diTPS may also be an enzyme catalysing the reaction IX:

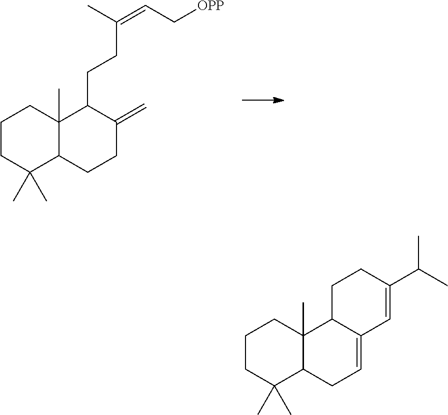

wherein OPP indicated diphosphate. During reaction IX the produced diterpene will in general maintain the stereochemistry around the decalin core found in the starting diterpene pyrophosphate intermediate.

The EpTPS8 like diTPS may also be an enzyme catalysing the reaction X:

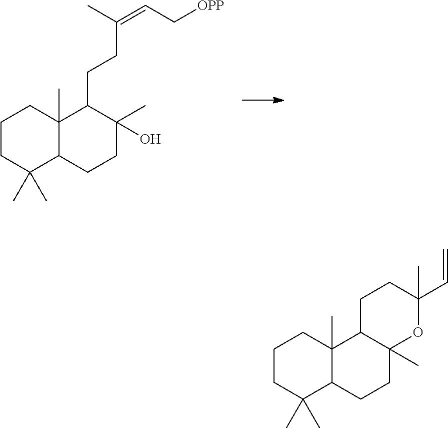

wherein —OPP indicated diphosphate. During reaction X the produced diterpene will in general maintain the stereochemistry around the decalin core found in the starting diterpene pyrophosphate intermediate.

In particular, the EpTPS8 like diTPS may be an enzyme catalysing the reaction XXV:

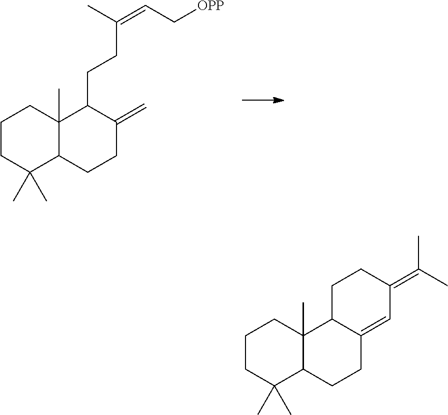

wherein —OPP indicates diphosphate. During reaction XXV the produced diterpene will in general maintain the stereochemistry around the decalin core found in the starting diterpene pyrophosphate intermediate.

In one embodiment EpTPS8 like diTPS may be a terpene synthase from Euphobia peplus, and in particular it may be TPS8 from Euphobia peplus. TPS8 from Euphobia peplus is also referred to as EpTPS herein. In particular, said EpTPS8 like diTPS may be a polypeptide of SEQ ID NO:9 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of EpTPS8 is a polypeptide, which is also capable of catalysing at least one of reactions VII, VIII, IX, X and XXV described above.

EpTPS23

The invention involves use of a diTPS of class I. In one embodiment said diTPS of class I may be an EpTPS23 like diTPS.

In particular, said diTPS of class I may be an EpTPS23 like diTPS in embodiments of the invention, wherein the diterpene to be produced contains a tricyclic ring structure. For example said diTPS of class I may be an EpTPS23 like diTPS in embodiments of the invention, wherein the diterpene to be produced contains a core of any of the formulas I and II:

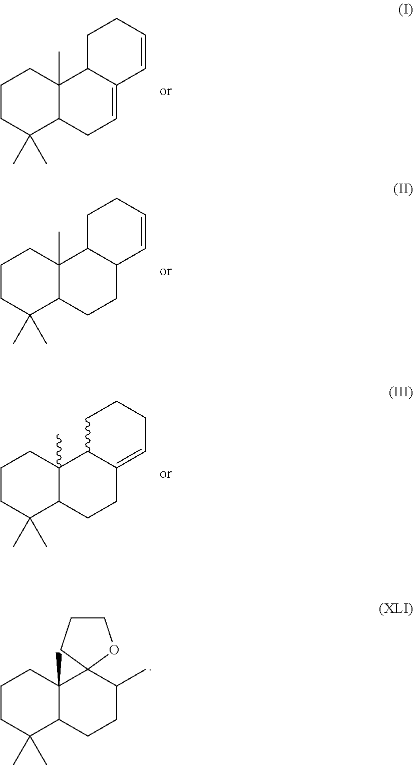

Dependent on the structure of the diterpene pyrophosphate intermediate then the diterpene containing a core of formula I or II may have different stereochemistry. In general the stereochemistry of the decalin core present in the diterpene pyrophosphate intermediate is maintained after the reaction catalysed by an EpTPS23 like diTPS.

The EpTPS23 like diTPS may in particular be an enzyme capable of catalysing the reaction XI:

Diterpene pyrophosphate intermediate containing a decalin core structure→Diterpene containing a core structure of formula I or formula II

In particular an EpTPS23 like diTPS may be an enzyme catalysing the reaction VIII:

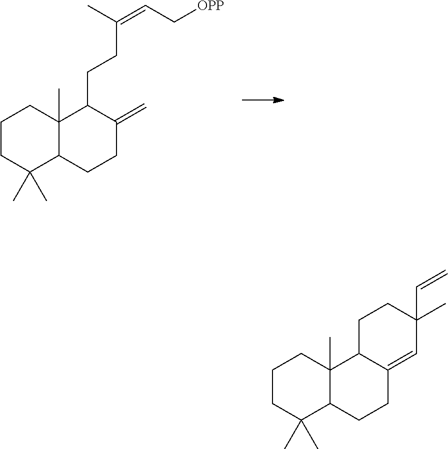

wherein —OPP indicated diphosphate. During reaction VIII the produced diterpene will in general maintain the stereochemistry around the decalin core found in the starting diterpene pyrophosphate intermediate.

The EpTPS23 like diTPS may also be an enzyme catalysing the reaction IX:

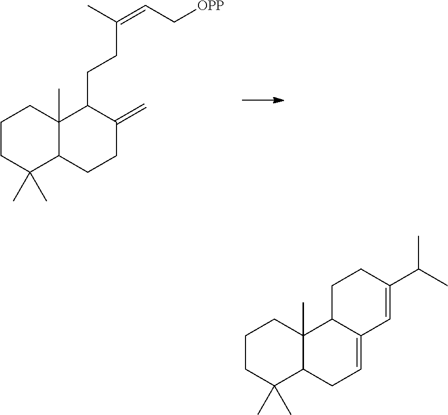

wherein —OPP indicated diphosphate. During reaction IX the produced diterpene will in general maintain the stereochemistry around the decalin core found in the starting diterpene pyrophosphate intermediate.

In one embodiment an EpTPS23 like diTPS may be a diterpene synthase from Euphobia peplus. In particular, the EpTPS23 like diTPS may be TPS23 of Euphobia peplus. TPS23 of Euphobia peplus may also be referred to as EpTPS23 herein. In particular, said EpTPS23 like diTPS may be a polypeptide of SEQ ID NO:10 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of EpTPS23 is a polypeptide, which is also capable of catalysing at least one of reactions VIII or IX described above.

SsSCS

The invention involves use of a diTPS of class I. In one embodiment said diTPS of class I may be a SsSCS like diTPS.

In particular, said diTPS of class I may be a SsSCS like diTPS in embodiments of the invention, wherein the diterpene to be produced contains a decalin substituted at the 10 position with C₅-alkenyl chain, which optionally may be substituted with a hydroxyl and/or a methyl group and/or ═C.

Furthermore, said diTPS of class I may be a SsSCS like diTPS in embodiments of the invention, wherein the diterpene to be produced contains a core of formula III, XXVI, XXVII, XXVIII, XXIX, XXX, XXXI, XXXII, XXXIII, or XXXIV:

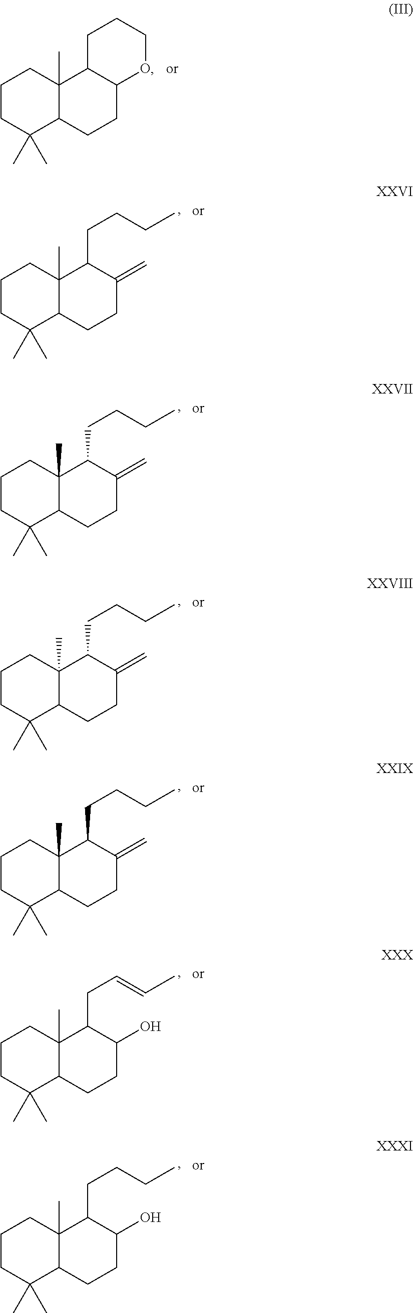

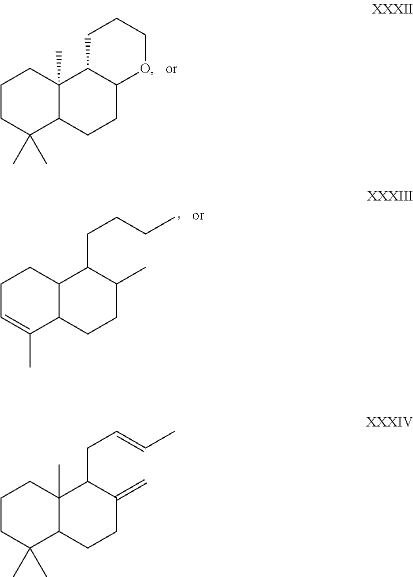

Dependent on the structure of the diterpene pyrophosphate intermediate then the diterpene containing a decalin substituted at the 10 position with said C₅-alkenyl chain, or the diterpene containing a core of formula III may have different stereochemistry. In general the stereochemistry of the decalin core present in the diterpene pyrophosphate intermediate is maintained after the reaction catalysed by a SsSCS like diTPS. The SsSCS like diTPS may be any enzyme capable of catalysing the following reaction XII:

Diterpene pyrophosphate intermediate containing a decalin core structure→Diterpene containing a decalin core substituted at the 10 position with C₅-alkenyl chain, which optionally may be substituted with a hydroxyl and/or a methyl group and/or ═C OR diterpene containing a core structure of formula III.

The SsSCS like diTPS may in particular be an enzyme capable of catalysing the reaction XVI:

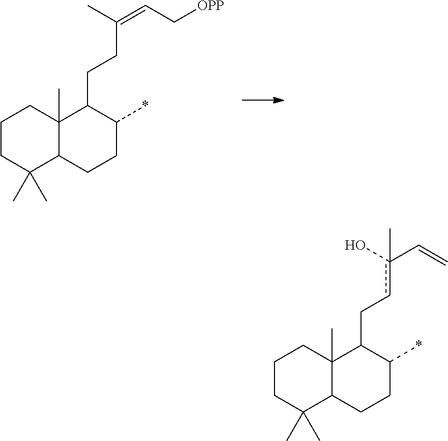

wherein —OPP is diphosphate; and [symbol]
indicates either a double bond or two single bonds, wherein one is substituted with —OH and the other with —CH₃; and
the dotted lines without star indicates a bond, which optionally is present.

Thus, [symbol] may be [symbol] or [symbol] .

It is to be understood that in embodiments of the invention, wherein the dotted line shown as [symbol] is not present, then also the hydroxyl group is not present. It is preferred that one and only one of the dotted lines without star indicates a bond.

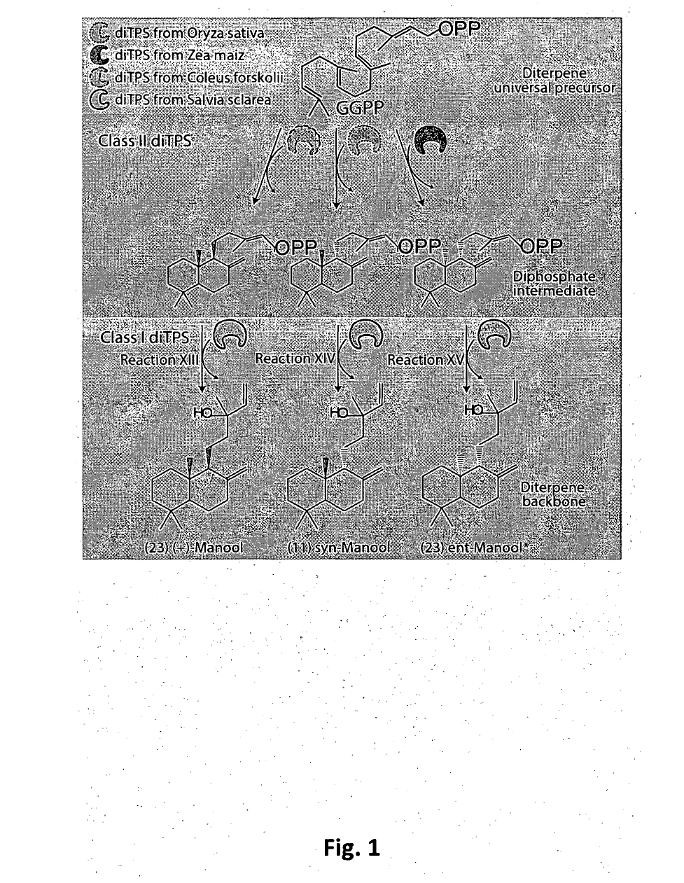

A SsSCS like diTPS may in particular be an enzyme capable of catalysing the reaction XVII:

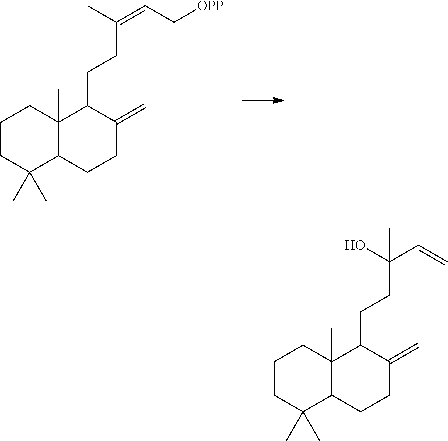

wherein OPP indicated diphosphate. During reaction XVII the produced diterpene will in general maintain the stereochemistry around the decalin core found in the starting diterpene pyrophosphate intermediate. Thus, the SsSCS like diTPS may be an enzyme catalysing any of the reactions XIII, XIV and XV shown in FIG. 1.

The SsSCS like diTPS may also be an enzyme catalysing the following reaction XXVIII:

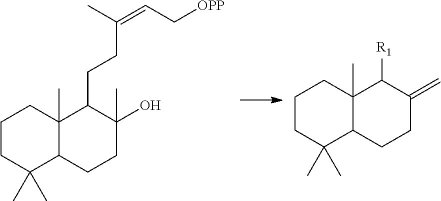

wherein OPP is diphosphate and R₁ is a C₅-alkenyl substituted with methyl and/or hydroxyl. Preferably, R₁ is C₅-alkenyl containing one or two double bonds. When R₁ is alkenyl containing one double bond, said alkenyl is preferably substituted with hydroxyl and methyl. When R₁ is alkenyl containing two double bonds, said alkenyl is preferably substituted with methyl.

The SsSCS like diTPS may also be an enzyme catalysing the following reaction XXIX:

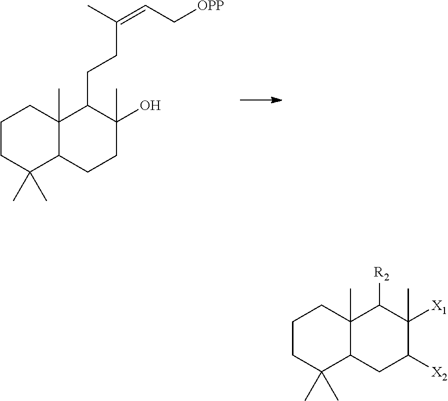

wherein —OPP is diphosphate and R₂ is a C₅-alkenyl substituted with methyl and/or hydroxyl or with ═C, and X₁ is either —OH or methyl, and X₂ is either —H or —OH, wherein one and only one of X₁ and X₂ is —OH. Preferably, R₂ is C₅-alkenyl containing one or two double bonds. When R₂ is alkenyl containing one double bond, said alkenyl is preferably substituted with hydroxyl and methyl or with ═C. When R₂ is alkenyl containing two double bonds, said alkenyl is preferably substituted with methyl.

The SsSCS like diTPS may also be an enzyme catalysing the reaction X:

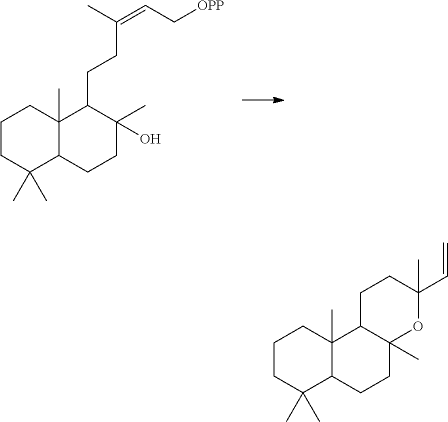

wherein OPP indicates diphosphate. During reaction X the produced diterpene will in general maintain the stereochemistry around the decalin core found in the starting diterpene pyrophosphate intermediate.

The SsSCS like diTPS may also be an enzyme catalysing the reaction XXX:

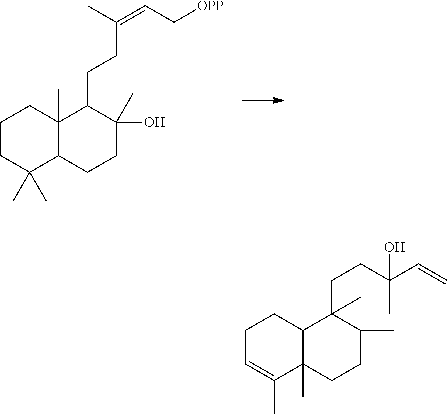

wherein OPP indicates diphosphate.

In one embodiment a SsSCS like diTPS may be SClareol Synthase (SCS) from Salvia Sclarea. SCS from Salvia Sclarea may also be referred to as SsSCS herein. In particular, said SsSCS like diTPS may be a polypeptide of SEQ ID NO:11 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of SsSCS is a polypeptide, which is also capable of catalysing at least one of reactions XII, XIII, XIV, XV, XVI, XVII, XXVIII, XXIX, or XXX described above.

CfTPS3

The invention involves use of a diTPS of class I. In one embodiment said diTPS of class I may be a CfTPS3 like diTPS. In embodiments of the invention, wherein the diTPS of class I is a CfTPS3 like diTPS, then it is preferred that the diTPS of class II is not CfTPS2 [SEQ ID NO:17], or SsLPPS [SEQ ID NO:6] or a functional homologue of any of the aforementioned sharing at least 70% sequence identity therewith. Thus, in embodiments of the invention, wherein the diTPS of class I is CfTPS3, then it is preferred that the diTPS of class II is not CfTPS2 or SsLPPS.

In particular, said diTPS of class I may be a CfTPS3 like diTPS in embodiments of the invention, wherein the diterpene to be produced contains a tricyclic ring structure. For example said diTPS of class I may be a CFTPS3 like diTPS in embodiments of the invention, wherein the diterpene to be produced contains a core of any of the formulas VI, IX, XXXV, XXXVI, II, XXXVII, XXXVIII, XXXIX, XL, III or XXXII:

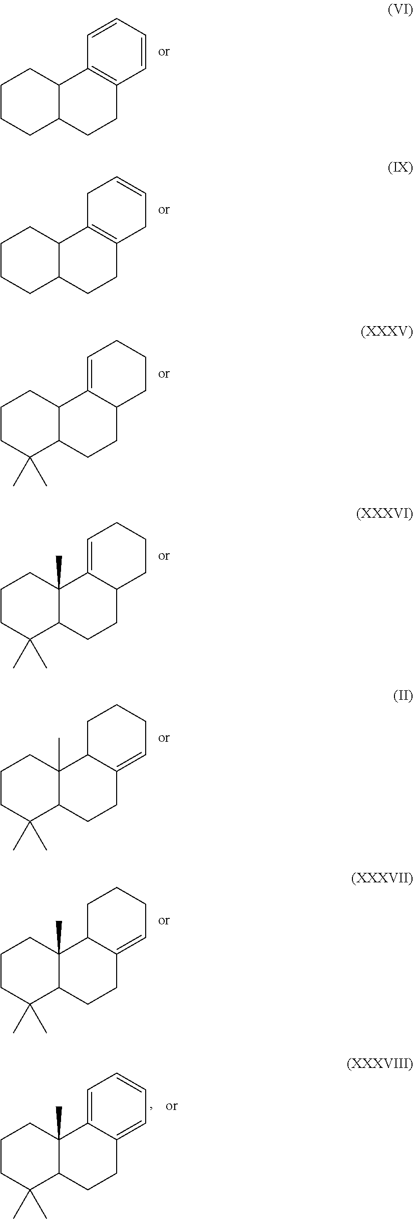

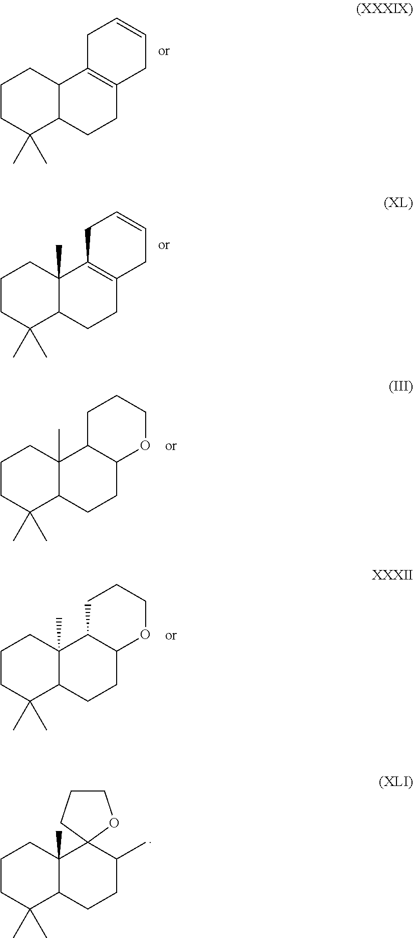

Dependent on the structure of the diterpene pyrophosphate intermediate then the diterpene containing a core of formula VI, IX, XXXV, II, or XXXIX may have different stereochemistry. In general the stereochemistry of the decalin core present in the diterpene pyrophosphate intermediate is maintained after the reaction catalysed by the CfTPS3 like diTPS.

The CfTPS3 like diTPS may be any enzyme capable of catalysing the reaction XXIII:

Diterpene pyrophosphate intermediate containing a decalin core structure→Diterpene containing a core structure of formula VI, formula IX, XXXV, XXXVI, II, XXXVII, XXXVIII, XXXIX, XL, III or XXXII.

The CfTPS3 like diTPS may in particular be an enzyme capable of catalysing the reaction XXIV:

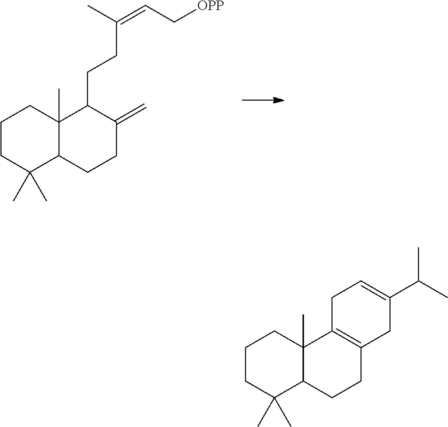

wherein OPP indicates diphosphate. During reaction XXIV the produced diterpene will in general maintain the stereochemistry around the decalin core found in the starting diterpene pyrophosphate intermediate.

The CfTPS3 like diTPS may in particular be an enzyme capable of catalysing the reaction XXII:

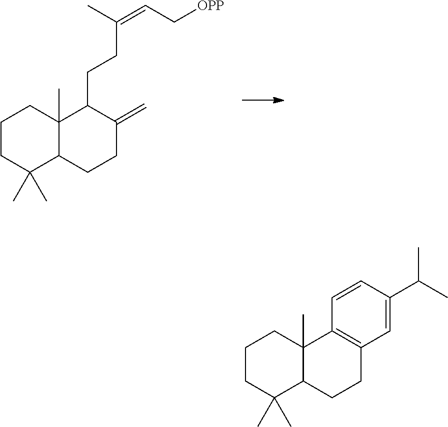

wherein OPP is diphosphate. During reaction XXII the produced diterpene will in general maintain the stereochemistry around the decalin core found in the starting diterpene pyrophosphate intermediate.

The CfTPS3 like diTPS may in particular be an enzyme capable of catalysing the reaction XXXI:

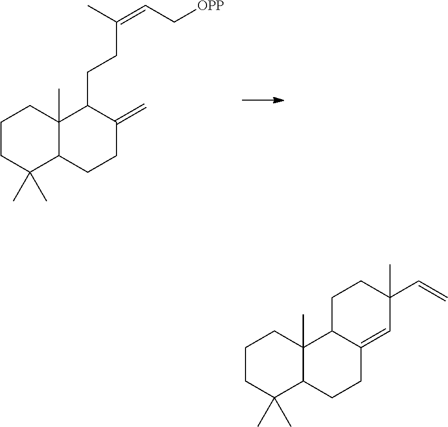

wherein OPP is diphosphate. During reaction XXXI the produced diterpene will in general maintain the stereochemistry around the decalin core found in the starting diterpene pyrophosphate intermediate.

The CfTPS3 like diTPS may in particular be an enzyme capable of catalysing the reaction XXXII:

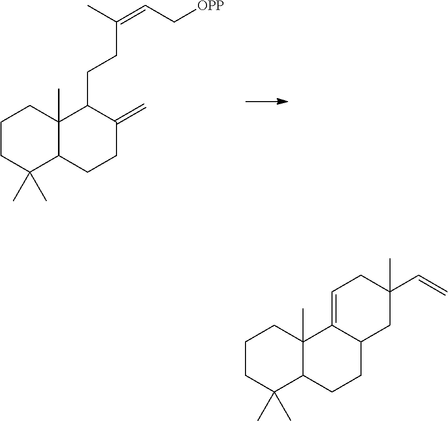

wherein OPP is diphosphate. During reaction XXXII the produced diterpene will in general maintain the stereochemistry around the decalin core found in the starting diterpene pyrophosphate intermediate.

The CfTPS3 like diTPS may also be an enzyme catalysing the reaction X:

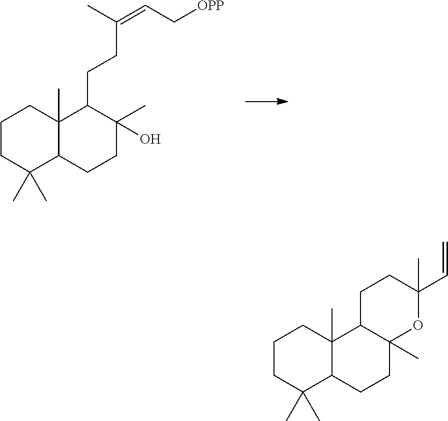

wherein OPP indicates diphosphate. During reaction X the produced diterpene will in general maintain the stereochemistry around the decalin core found in the starting diterpene pyrophosphate intermediate.

In one embodiment the CfTPS3 like diTPS may be a diterpene synthase from Coleus forskohlii. In particular, the CfTPS3 like diTPS may be a TPS3 from Coleus forskohlii. TPS3 from Coleus forskohlii may also be referred to as CfTPS3. In particular, said CfTPS3 like diTPS may be a polypeptide of SEQ ID NO:12 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of CfTPS3 is a polypeptide, which is also capable of catalysing at least one of reactions XXII, XXIII or XXIV described above.

CfTPS4

The invention involves use of a diTPS of class I. In one embodiment said diTPS of class I may be a CfTPS4 like diTPS. In embodiments of the invention, wherein the diTPS of class I is a CfTPS4 like diTPS, then it is preferred that the diTPS of class II is not CfTPS2[SEQ ID NO:17], or SsLPPS [SEQ ID NO:6] or a functional homologue of any of the aforementioned sharing at least 70% sequence identity therewith. Thus, in embodiments of the invention, wherein the diTPS of class I is CfTPS4, then it is preferred that the diTPS of class II is not CfTPS2 or SsLPPS.

In particular, said diTPS of class I may be a CfTPS4 like diTPS in embodiments of the invention, wherein the diterpene to be produced contains a tricyclic ring structure. For example said diTPS of class I may be a CfTPS4 like diTPS in embodiments of the invention, wherein the diterpene to be produced contains a core of any of the formulas VI, IX, XXXV, XXXVI, II, XXXVII, XXXVIII, XXXIX or XL:

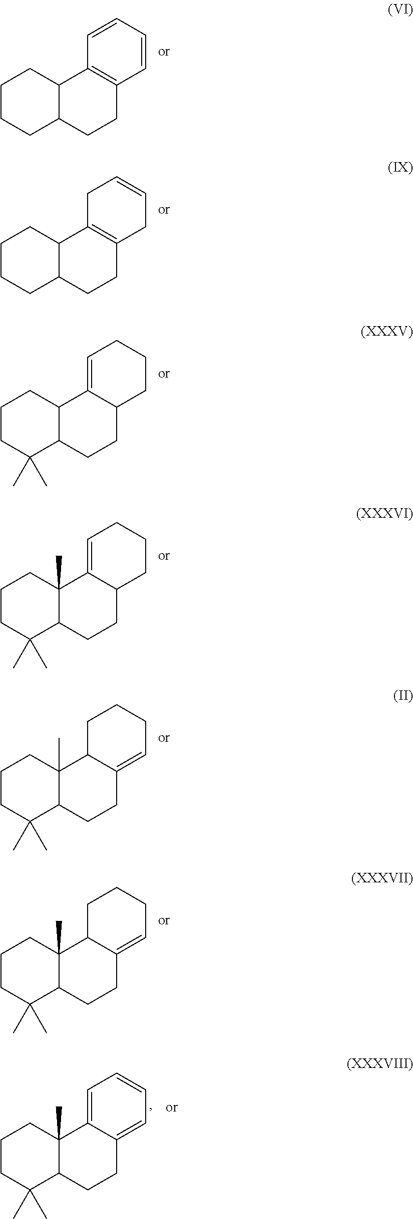

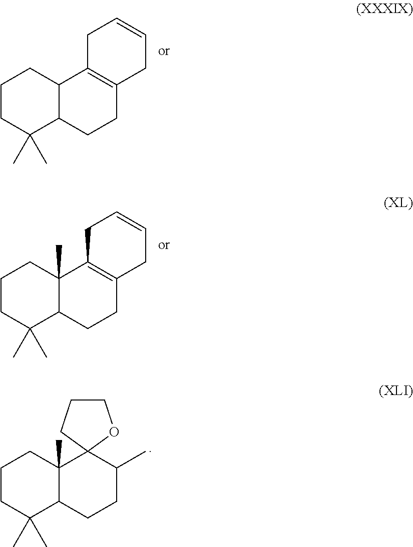

Dependent on the structure of the diterpene pyrophosphate intermediate then the diterpene containing a core of formula VI, IX, XXXV, II, or XXXIX, may have different stereochemistry. In general the stereochemistry of the decalin core present in the diterpene pyrophosphate intermediate is maintained after the reaction catalysed by the CfTPS4 like diTPS.

The CfTPS4 like diTPS may be any enzyme capable of catalysing the reaction XXIII:

Diterpene pyrophosphate intermediate containing a decalin core structure→Diterpene containing a core structure of formula VI, IX, XXXV, XXXVI, II, XXXVII, XXXVIII, XXXIX or XL.

The CfTPS4 like diTPS may in particular be an enzyme capable of catalysing the reaction XXIV:

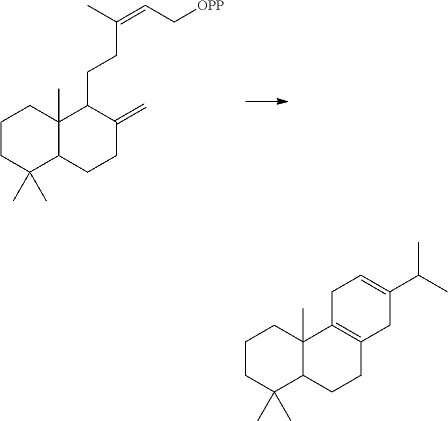

wherein OPP indicates diphosphate. During reaction XXIV the produced diterpene will in general maintain the stereochemistry around the decalin core found in the starting diterpene pyrophosphate intermediate.

The CfTPS4 like diTPS may in particular be an enzyme capable of catalysing the reaction XXII:

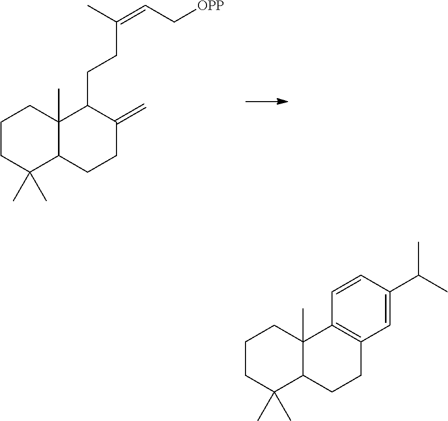

wherein OPP is diphosphate. During reaction XXII the produced diterpene will in general maintain the stereochemistry around the decalin core found in the starting diterpene pyrophosphate intermediate.

The CfTPS4 like diTPS may in particular be an enzyme capable of catalysing the reaction XXXI:

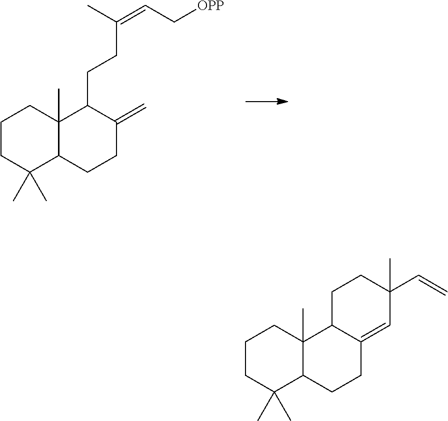

wherein OPP is diphosphate. During reaction XXXI the produced diterpene will in general maintain the stereochemistry around the decalin core found in the starting diterpene pyrophosphate intermediate.

The CfTPS4 like diTPS may in particular be an enzyme capable of catalysing the reaction XXXII:

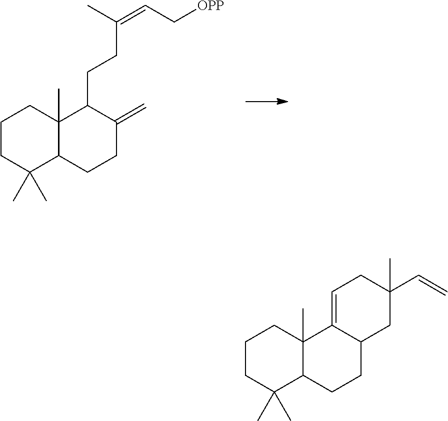

wherein OPP is diphosphate. During reaction XXXII the produced diterpene will in general maintain the stereochemistry around the decalin core found in the starting diterpene pyrophosphate intermediate.

In one embodiment the CfTPS4 like diTPS may be a diterpene synthase from Coleus forskohlii. In particular, the CfTPS4 like diTPS may be a TPS4 from Coleus forskohlii. TPS4 from Coleus forskohlii may also be referred to as CfTPS4. In particular, said CfTPS4 like diTPS may be a polypeptide of SEQ ID NO:13 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of CfTPS4 is a polypeptide, which is also capable of catalysing at least one of reactions XXII, XXIII or XXIV described above.

TwTPS2

The invention involves use of a diTPS of class I. In one embodiment said diTPS of class I may be a TwTPS2 like diTPS.

In particular, said diTPS of class I may be a TwTPS2 like diTPS in embodiments of the invention, wherein the diterpene to be produced contains a tricyclic ring structure. For example said diTPS of class I may be a TwTPS2 like diTPS in embodiments of the invention, wherein the diterpene to be produced contains a core of any of the formulas IV, V or X:

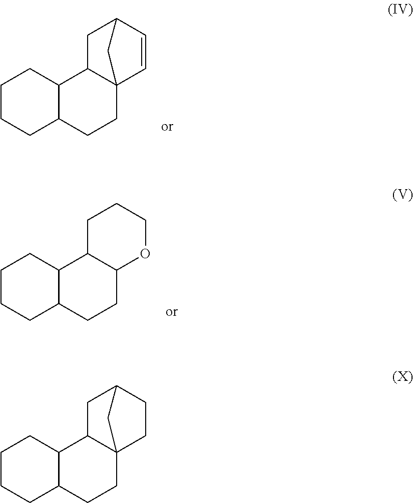

Dependent on the structure of the diterpene pyrophosphate intermediate then the diterpene containing a core of formula IV and V, may have different stereochemistry. In general the stereochemistry of the decalin core present in the diterpene pyrophosphate intermediate is maintained after the reaction catalysed by the TwTPS2 like diTPS.

The TwTPS2 like diTPS may be any enzyme capable of catalysing the reaction XXVI:

Diterpene pyrophosphate intermediate containing a decalin core structure→Diterpene containing a core structure of formula IV or formula V or formula X

The TwTPS2 like diTPS may be any enzyme capable of catalysing conversion of a diterpene pyrophosphate intermediate to a diterpene containing a core of either formula IV or V. The TwTPS2 like diTPS may in particular be an enzyme capable of catalysing the reaction XIX:

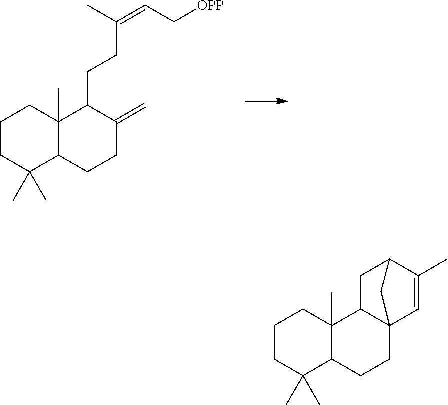

wherein OPP is diphosphate. During reaction XIX the produced diterpene will in general maintain the stereochemistry around the decalin core found in the starting diterpene pyrophosphate intermediate.

The TwTPS2 like diTPS may in particular be an enzyme capable of catalysing the reaction XXVII:

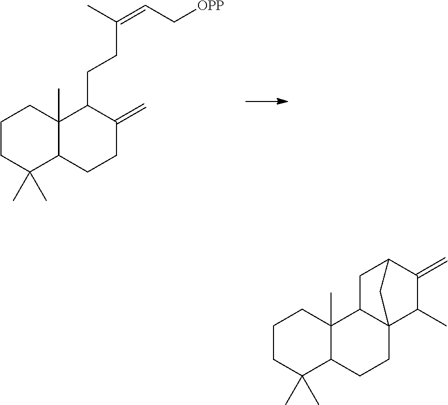

wherein OPP is diphosphate. During reaction XIX the produced diterpene will in general maintain the stereochemistry around the decalin core found in the starting diterpene pyrophosphate intermediate.

The TwTPS2 like diTPS may in particular be an enzyme capable of catalysing the reaction XX:

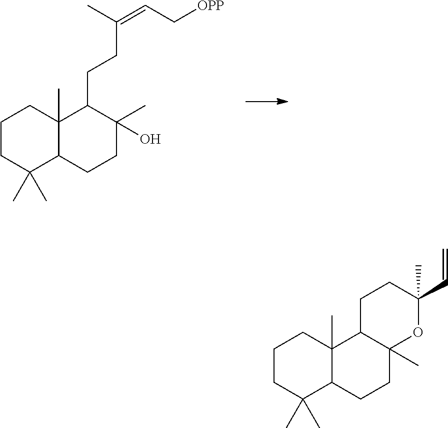

wherein OPP indicated diphosphate. During reaction XX the produced diterpene will in general maintain the stereochemistry around the decalin core found in the starting diterpene pyrophosphate intermediate.

In one embodiment the TwTPS2 like diTPS may be a diterpene synthase from Tripterygium Wilfordii. In particular, the TwTPS2 like diTPS may be a TPS2 from Tripterygium Wilfordii. TPS2 from Tripterygium Wilfordii may also be referred to as TwTPS2. In particular, said TwTPS2 like diTPS may be a polypeptide of SEQ ID NO:14 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of TwTPS2 is a polypeptide, which is also capable of catalysing at least one of reactions, XIX, XX, XXVI or XXVII described above.

EpTPS1

The invention involves use of a diTPS of class I. In one embodiment said diTPS of class I may be an EpTPS1 like diTPS.

In particular, said diTPS of class I may be an EpTPS1 like diTPS in embodiments of the invention, wherein the diterpene to be produced contains a tricyclic ring structure. For example said diTPS of class I may be an EpTPS1 like diTPS in embodiments of the invention, wherein the diterpene to be produced contains a core of any of the formulas IV or V:

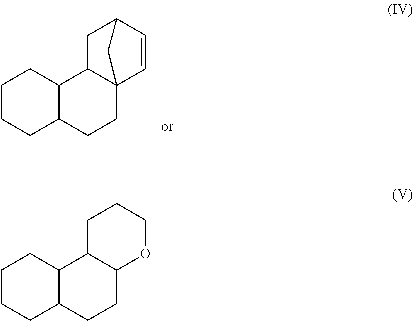

Dependent on the structure of the diterpene pyrophosphate intermediate then the diterpene containing a core of formula IV and V, may have different stereochemistry. In general the stereochemistry of the decalin core present in the diterpene pyrophosphate intermediate is maintained after the reaction catalysed by the EpTPS1 like diTPS.

The EpTPS1 like diTPS may be any enzyme capable of catalysing the reaction XVIII:

Diterpene pyrophosphate intermediate containing a decalin core structure→Diterpene containing a core structure of formula IV or formula V

The EpTPS1 like diTPS may be any enzyme capable of catalysing conversion of a diterpene pyrophosphate intermediate to a diterpene containing a core of either formula IV or V. The EpTPS1 like diTPS may in particular be an enzyme capable of catalysing the reaction XIX:

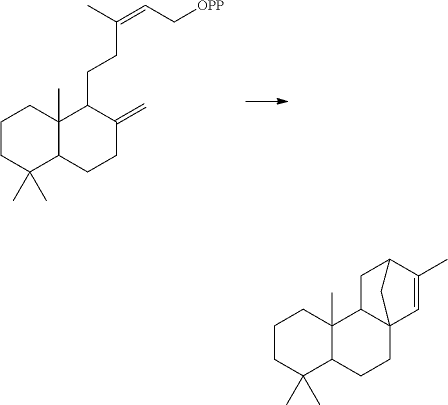

wherein OPP is diphosphate. During reaction XIX the produced diterpene will in general maintain the stereochemistry around the decalin core found in the starting diterpene pyrophosphate intermediate.

The EpTPS1 like diTPS may in particular be an enzyme capable of catalysing the reaction XX:

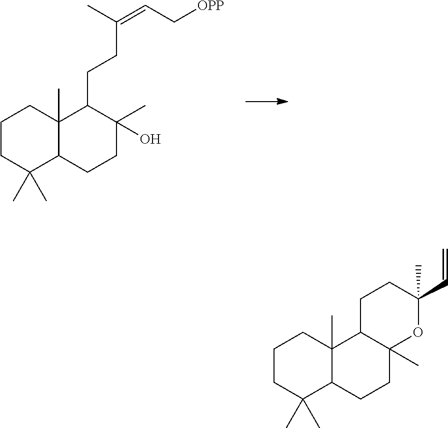

wherein OPP indicated diphosphate. During reaction XX the produced diterpene will in general maintain the stereochemistry around the decalin core found in the starting diterpene pyrophosphate intermediate.

In one embodiment the EpTPS1 like diTPS may be a diterpene synthase from Euphobia peplus. In particular, the EpTPS1 like diTPS may be a TPS1 from Euphobia peplus. TPS1 from Euphobia peplus may also be referred to as EpTPS1. In particular, said EpTPS1 like diTPS may be a polypeptide of SEQ ID NO:15 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of EpTPS1 is a polypeptide, which is also capable of catalysing at least one of reactions XVIII, XIX or XX described above.

MvTPS5

The invention involves use of a diTPS of class I. In one embodiment said diTPS of class I may be a MvTPS5 like diTPS.

In particular, said diTPS of class I may be a MvTPS5 like diTPS in embodiments of the invention, wherein the diterpene to be produced contains a tricyclic ring structure. For example said diTPS of class I may be a MvTPS5 like diTPS in embodiments of the invention, wherein the diterpene to be produced contains a core of any of the formulas VI, IX, XXXV, XXXVI, II, XXXVII, XXXVIII, XXXIX, XL, III or XXXII:

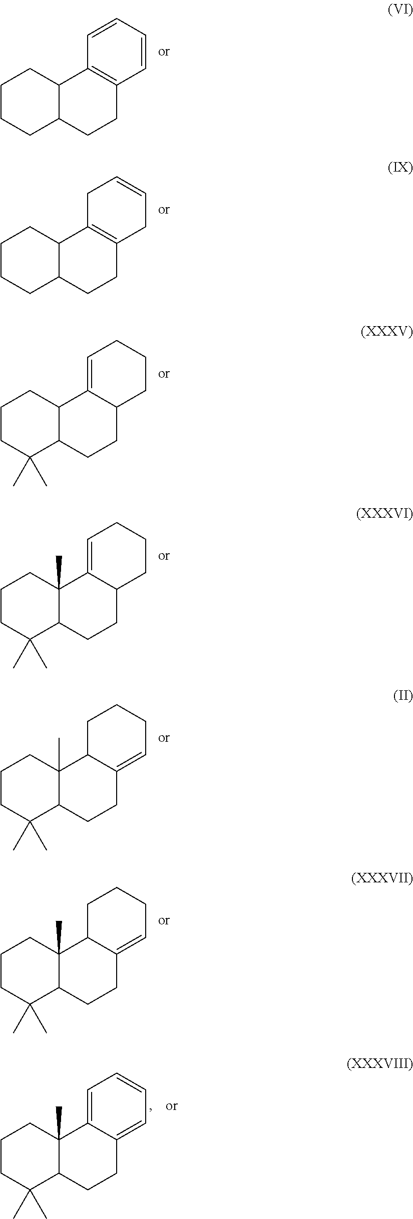

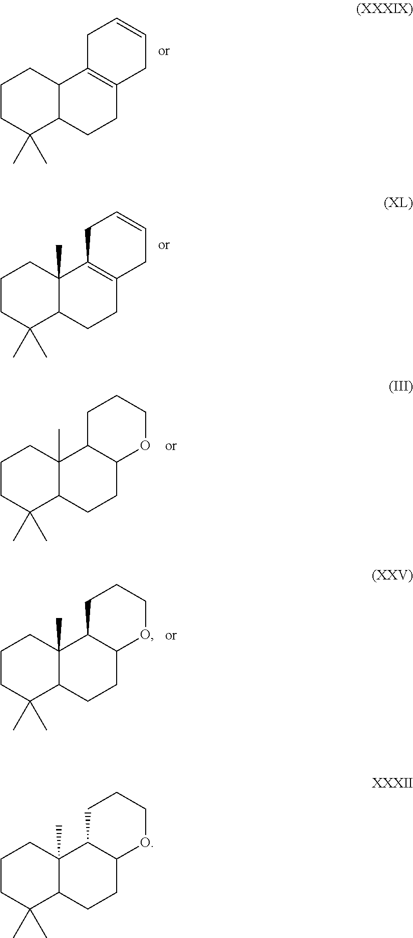

Dependent on the structure of the diterpene pyrophosphate intermediate then the diterpene containing a core of formula VI, IX, XXXV, II, XXXIX or III, may have different stereochemistry. In general the stereochemistry of the decalin core present in the diterpene pyrophosphate intermediate is maintained after the reaction catalysed by the MvTPS5 like diTPS.

The MvTPS5 like diTPS may be any enzyme capable of catalysing the reaction XXIII:

Diterpene pyrophosphate intermediate containing a decalin core structure→Diterpene containing a core structure of formula VI, IX, XXXV, XXXVI, II, XXXVII, XXXVIII, XXXIX, XL, III or XXXII.

The MvTPS5 like diTPS may in particular be an enzyme capable of catalysing the reaction XXIV:

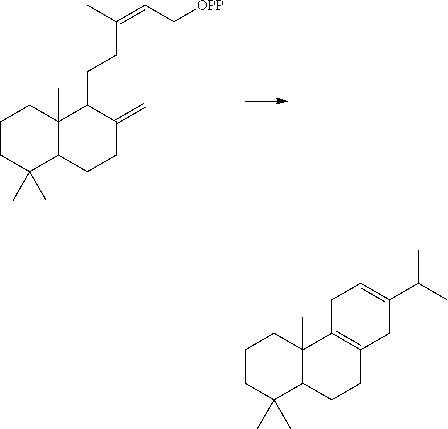

wherein OPP indicates diphosphate. During reaction XXIV the produced diterpene will in general maintain the stereochemistry around the decalin core found in the starting diterpene pyrophosphate intermediate.

The MvTPS5 like diTPS may in particular be an enzyme capable of catalysing the reaction XXII:

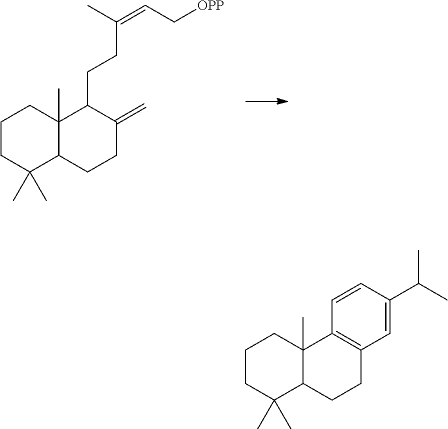

wherein OPP is diphosphate. During reaction XXII the produced diterpene will in general maintain the stereochemistry around the decalin core found in the starting diterpene pyrophosphate intermediate.

The MvTPS5 like diTPS may in particular be an enzyme capable of catalysing the reaction XXXI:

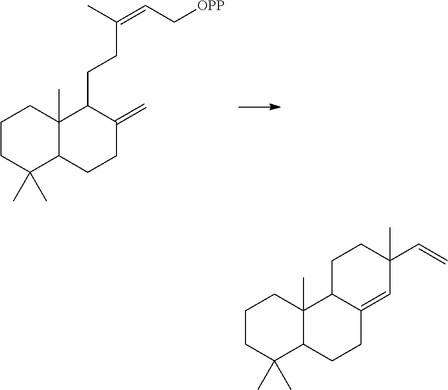

wherein OPP is diphosphate. During reaction XXXI the produced diterpene will in general maintain the stereochemistry around the decalin core found in the starting diterpene pyrophosphate intermediate.

The MvTPS5 like diTPS may in particular be an enzyme capable of catalysing the reaction XXXII:

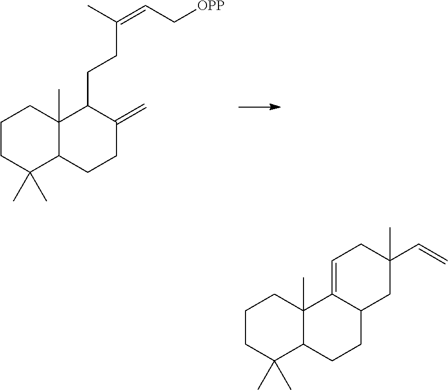

wherein OPP is diphosphate. During reaction XXXII the produced diterpene will in general maintain the stereochemistry around the decalin core found in the starting diterpene pyrophosphate intermediate.

The MvTPS5 like diTPS may also be an enzyme catalysing the reaction X:

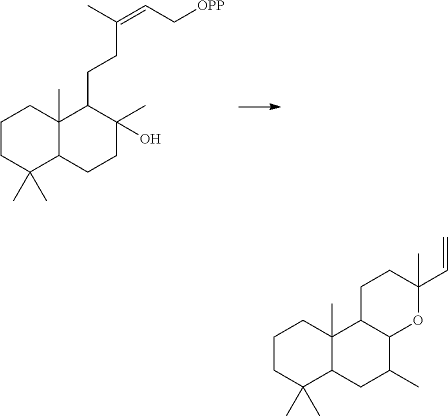

wherein OPP indicates diphosphate. During reaction X the produced diterpene will in general maintain the stereochemistry around the decalin core found in the starting diterpene pyrophosphate intermediate.

In one embodiment the MvTPS5 like diTPS may be a diterpene synthase from Marrubium vulgare. In particular, the MvTPS5 like diTPS may be a TPS5 from Marrubium vulgare. TPS5 from Marrubium vulgare may also be referred to as MvTPS5. In particular, said MvTPS5 like diTPS may be a polypeptide of SEQ ID NO:18 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of MvTPS5 is a polypeptide, which is also capable of catalysing at least one of reactions XXII, XXIII or XXIV described above.

CfTPS14

The invention involves use of a diTPS of class I. In one embodiment said diTPS of class I may be an CfTPS14 like diTPS.

In particular, said diTPS of class I may be an CfTPS14 like diTPS in embodiments of the invention, wherein the diterpene to be produced contains a tricyclic ring structure. For example said diTPS of class I may be an CfTPS14 like diTPS in embodiments of the invention, wherein the diterpene to be produced contains a core of any of the formulas IV or V:

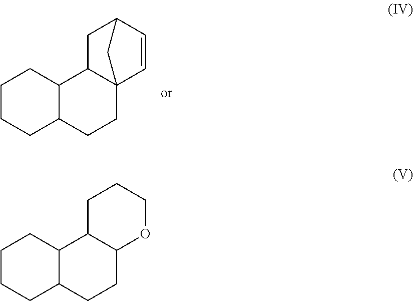

Dependent on the structure of the diterpene pyrophosphate intermediate then the diterpene containing a core of formula IV and V, may have different stereochemistry. In general the stereochemistry of the decalin core present in the diterpene pyrophosphate intermediate is maintained after the reaction catalysed by the CfTPS14 like diTPS.

The CfTPS14 like diTPS may be any enzyme capable of catalysing the reaction XVIII:

Diterpene pyrophosphate intermediate containing a decalin core structure→Diterpene containing a core structure of formula IV or formula V

The CfTPS14 like diTPS may be any enzyme capable of catalysing conversion of a diterpene pyrophosphate intermediate to a diterpene containing a core of either formula IV or V. The CfTPS14 like diTPS may in particular be an enzyme capable of catalysing the reaction XIX:

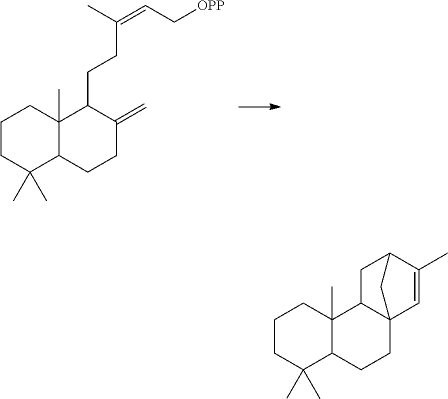

wherein OPP is diphosphate. During reaction XIX the produced diterpene will in general maintain the stereochemistry around the decalin core found in the starting diterpene pyrophosphate intermediate.

The CfTPS14 like diTPS may in particular be an enzyme capable of catalysing the reaction XX:

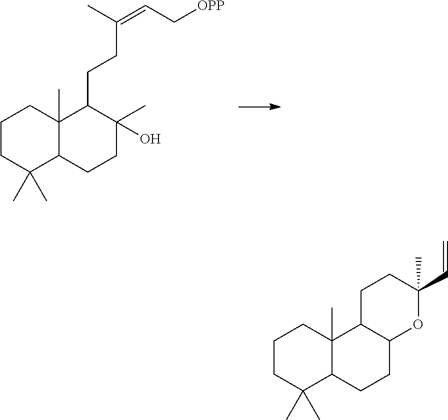

wherein OPP indicated diphosphate. During reaction XX the produced diterpene will in general maintain the stereochemistry around the decalin core found in the starting diterpene pyrophosphate intermediate.

In one embodiment the CfTPS14 like diTPS may be a diterpene synthase from Coleus forskohlii. In particular, the CfTPS14 like diTPS may be a TPS14 from Coleus forskohlii. TPS14 from Coleus forskohlii may also be referred to as CfTPS14. In particular, said CfTPS14 like diTPS may be a polypeptide of SEQ ID NO:16 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of CfTPS14 is a polypeptide, which is also capable of catalysing at least one of reactions XVIII, XIX or XX described above.

Additional Recombinant Modifications

The host organisms according to the present invention may also be recombinantly modified in addition to comprising the heterologous nucleic acids encoding a diTPS of class I and a diTPS of class II as described herein.

For example the host organism may be modified to increase the pool of GGPP. As described herein elsewhere, GGPP is the starting compound for production of diterpenes. Thus, if the host organism is modified to increase the pool of GGPP, then frequently, the host organism will be capable of producing increased amounts of diterpene.

Various methods for increasing the pool of GGPP are well known in the art. These includes methods of reducing the activity of enzymes reducing the level of GGPP.

In one embodiment the pool of GGPP is increased by expression of one or more enzymes involved in synthesis of GGPP.

Thus, it may be preferred that the host organism comprises a heterologous nucleic acid encoding GGPP synthase (GGPPS). Said GGPPS may be any GGPPS, e.g. BTS1 of S. cerevisiae.

In particular, the GGPPS may be the GGPPS described by Zhou, Y. J., W. Gao, Q. Rong, G. Jin, H. Chu, W. Liu, W. Yang, Z. Zhu, G. Li, G. Zhu, L. Huang and Z. K. Zhao (2012). “Modular Pathway Engineering of Diterpenoid Synthases and the Mevalonic Acid Pathway for Miltiradiene Production.” Journal of the American Chemical Society 134(6): 3234-3241.

Accordingly, the host organism may express a fusion of SmCPS and SmKSL, and/or a fusion of BTS1 (GGPP synthase) and ERG20 (farnesyl diphosphate synthase) as described in Zhou et al., 2012.

The host organism may also comprise a heterologous nucleic acid encoding a GGPPS from a plant, e.g. from Coleus forskohlii. Thus, in one embodiment the host organism comprises:
a) a heterologous nucleic acid encoding Coleus forskohlii deoxyxylulose 5-phosphate synthase (CfDXS) of SEQ ID NO:26 or a functional homologue of any of the aforementioned sharing at least 70%, such as at least 80%, such as at least 85%, such as at least 90%, such as at least 95%, such as at least 98%, such as at least 99% sequence identity therewith and/or
b) a heterologous nucleic acid encoding Coleus forskohlii geranylgeranylpyrophosphate synthase (CfGGPPs) of SEQ ID NO:27 or a functional homologue of any of the aforementioned sharing at least 70%, such as at least 80%, such as at least 85%, such as at least 90%, such as at least 95%, such as at least 98%, such as at least 99% sequence identity therewith.

Production of Kolavelool

It is one aspect of the invention to provide methods for producing kolavelool. In particular, the invention provides methods for producing kolavelool, said methods comprising the steps of:
a) providing a host organism comprising
   I. a heterologous nucleic acid encoding a diTPS of class II, which is an CLPP like type diTPS; and
   II. A heterologous nucleic acid encoding diTPS of class I,
b) Incubating said host organism in the presence of geranylgeranyl pyrophosphate (GGPP) under conditions allowing growth of said host organism;
c) Optionally isolating kolavelool from the host organism.

Said host organism may for example be any of the host organisms described herein in the section “Host organism”.

Said CLPP type diTPS may be any of the CLPP type diTPS described herein in the section “LPP type diTPS”. In particular the LPP type diTPS may be TwTPS14/28 of SEQ ID NO:8 or a functional homologue thereof sharing at least 70%, such as at least 80%, such as at least 85%, such as at least 90%, such as at least 95%, such as at least 98%, such as at least 99% sequence identity therewith. Said functional homologue is preferably an enzyme capable of catalysing reaction XXXV.

The diTPS of class I may be any diTPS of class I, such as any of he diTPS of class I described herein. In particular, said diTPS of class I may be a diTPS of class I capable of catalysing the reaction XXXVII:

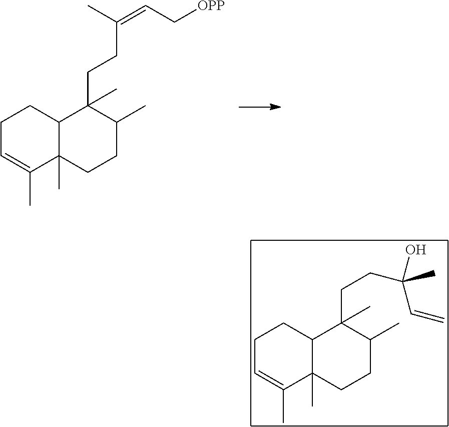

In one preferred embodiment of the invention, the diTPS of class I may in embodiment be a SsSCS like diTPS, for example any of the SsSCS like diTPS described herein in the section “ScSCS”. In particular the SsSCS like diTPS may be SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.

Sequence Identity

A high level of sequence identity indicates likelihood that the first sequence is derived from the second sequence. Amino acid sequence identity requires identical amino acid sequences between two aligned sequences. Thus, a candidate sequence sharing 80% amino acid identity with a reference sequence, requires that, following alignment, 80% of the amino acids in the candidate sequence are identical to the corresponding amino acids in the reference sequence. Identity according to the present invention is determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. The ClustalW software is available from as a ClustalW WWW Service at the European Bioinformatics Institute http://www.ebi.ac.uk/clustalw or via, the software BioEdit. Using this program with its default settings, the mature (bioactive) part of a query and a reference polypeptide are aligned. The number of fully conserved residues are counted and divided by the length of the reference polypeptide. Thus, sequence identity is calculated over the entire length of the reference polypeptide.

The ClustalW algorithm may similarly be used to align nucleotide sequences. Sequence identities may be calculated in a similar way as indicated for amino acid sequences.

In one important embodiment, the cell of the present invention comprises a nucleic acid sequence coding, as define herein.

Heterologous Nucleic Acid

The term “heterologous nucleic acid” as used herein refers to a nucleic acid sequence, which has been introduced into the host organism, wherein said host does not endogenously comprise said nucleic acid. For example, said heterologous nucleic acid may be introduced into the host organism by recombinant methods. Thus, the genome of the host organism has been augmented by at least one incorporated heterologous nucleic acid sequence. It will be appreciated that typically the genome of a recombinant host described herein is augmented through the stable introduction of one or more heterologous nucleic acids encoding one or more diTPS's.

Suitable host organisms include microorganisms, plant cells, and plants, and may for example be any of the host organisms described herein below in the section “Host organism”.

In general the heterologous nucleic acid encoding a polypeptide (also referred to as “coding sequence” in the following) is operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned at further distance, for example as much as about 5,000 nucleotides upstream of the translation initiation site, or about 2,000 nucleotides upstream of the transcription start site.

The choice of regulatory regions to be included depends upon several factors, including the type of host organism. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host organisms obtained, using appropriate codon bias tables for that host (e.g., microorganism). Nucleic acids may also be optimized to a GC-content preferable to a particular host, and/or to reduce the number of repeat sequences. As isolated nucleic acids, these modified sequences can exist as purified molecules and can be incorporated into a vector or a virus for use in constructing modules for recombinant nucleic acid constructs.

Diterpene Pyrophosphate Intermediate

The term “decalin” as used herein refers to a compound of the formula VII:

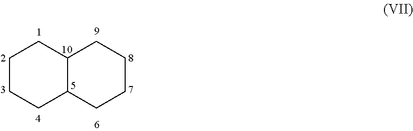

The numbering of carbon atoms provided in formula VII is adhered to throughout this description.

A compound containing or comprising a “decalin core” as used herein refers to a compound comprising above mentioned structure of formula VII, wherein each of the carbon atoms numbered 1 to 10 may be substituted with one or two substituents. It is possible that two of said substituents are fused to form a ring, and thus compound containing or comprising decalin may contain 3 or more rings.

The term “diterpene pyrophosphate intermediate” as used herein refers to a compound, which is the product of bicyclisation of GGPP in a reaction catalysed by a diTPS class II enzyme. The diterpene pyrophosphate intermediate according to the invention contains a decalin core, and comprises a pyrophosphate group.

It is preferred that the diterpene pyrophosphate intermediate of the invention is a compound containing a decalin core, which is substituted at one of more positions with substituents selected from the group consisting of alkyl, alkenyl and hydroxyl, wherein one of said alkyl or alkenyl is substituted with O-pyrophosphate.

The terms “diphosphate” and “pyrophosphate” are used interchangeably herein. The abbreviation “OPP”, “—OPP” or “PPO—” as used herein refers to diphosphate.

The term “alkyl” as used herein refers to a saturated, straight or branched hydrocarbon chain. The hydrocarbon chain preferably contains of from one to eighteen carbon atoms (C₁₋₁₈-alkyl), more preferred of from one to six carbon atoms (C₁₋₆-alkyl), including methyl, ethyl, propyl, isopropyl, butyl, isobutyl, secondary butyl, tertiary butyl, pentyl, isopentyl, neopentyl, tertiary pentyl, hexyl and isohexyl.

The term “alkenyl” as used herein refers to a saturated, straight or branched hydrocarbon chain containing at least one double bond. Alkenyl may preferably be any of the alkyls described above containing one or more double bonds.

In particular, the diterpene pyrophosphate intermediate of the invention is a compound containing a decalin core, wherein said decalin is
i. substituted at the 4 position with one or two alkyl, such as with two alkyl, wherein said alkyl for example may be C₁₋₃, alkyl, for example said alkyl may be methyl;
ii. substituted at the 8 position with one or two substituents individually selected from the group consisting of alkyl, hydroxyl and alkenyl, wherein said alkyl for example may be C₁₋₃ alkyl, for example said alkyl may be methyl, and said alkenyl may be C₁₋₃ alkenyl, for example said alkenyl may be ═C;
iii. substituted at the 9 position with alkenyl-O—PP, wherein said alkenyl for example may be branched C4-8-alkenyl, such as branched C5-7-alkenyl, for example branched C6-alkenyl; and
iv. substituted at the 10 position with alkyl, wherein said alkyl for example may be C₁₋₃, alkyl, for example said alkyl may be methyl.

In particular, the substituent at the 9 position may be alkenyl of formula VIII:

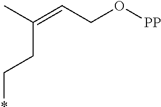

wherein the asterisk indicates the point of attachment to the decalin core.

It is also preferred that the stereochemistry around substituents 9 and 10 is predetermined. Thus, said diterpene pyrophosphate intermediate may contain a decalin core substituted as indicated above, wherein the substitutions at the 9 and 10 positions are (9R, 10R), (9S,10S), (9S, 10R) or (9R, 10S), for example the substitutions at the 9 and 10 positions are (9R, 10R), (9S,10S) or (9S, 10R).

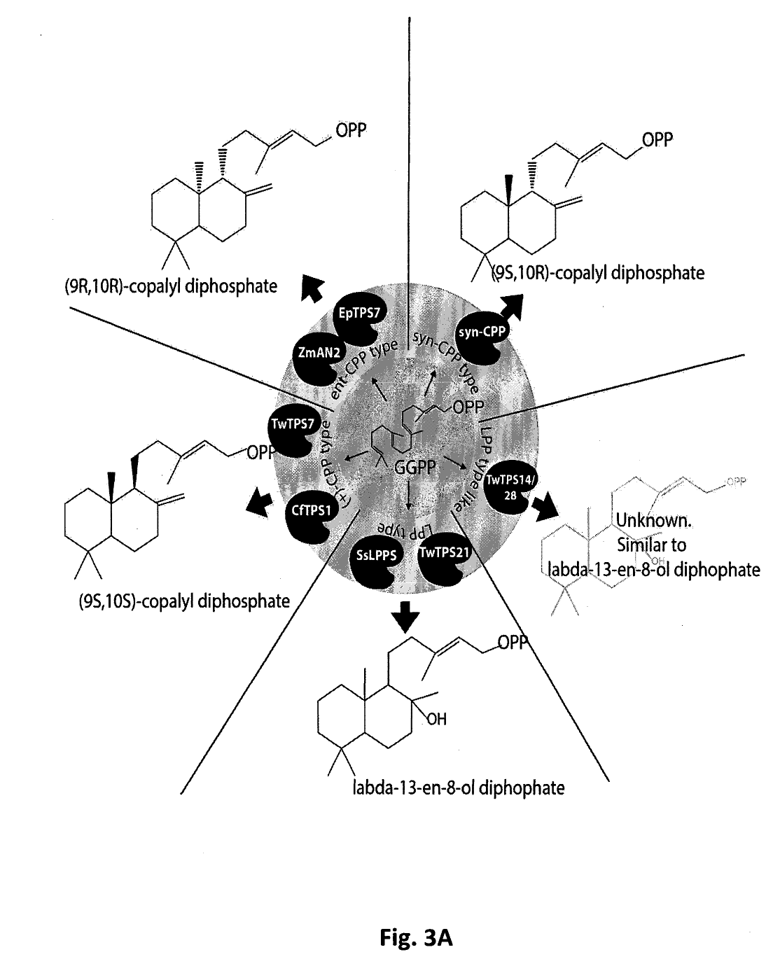

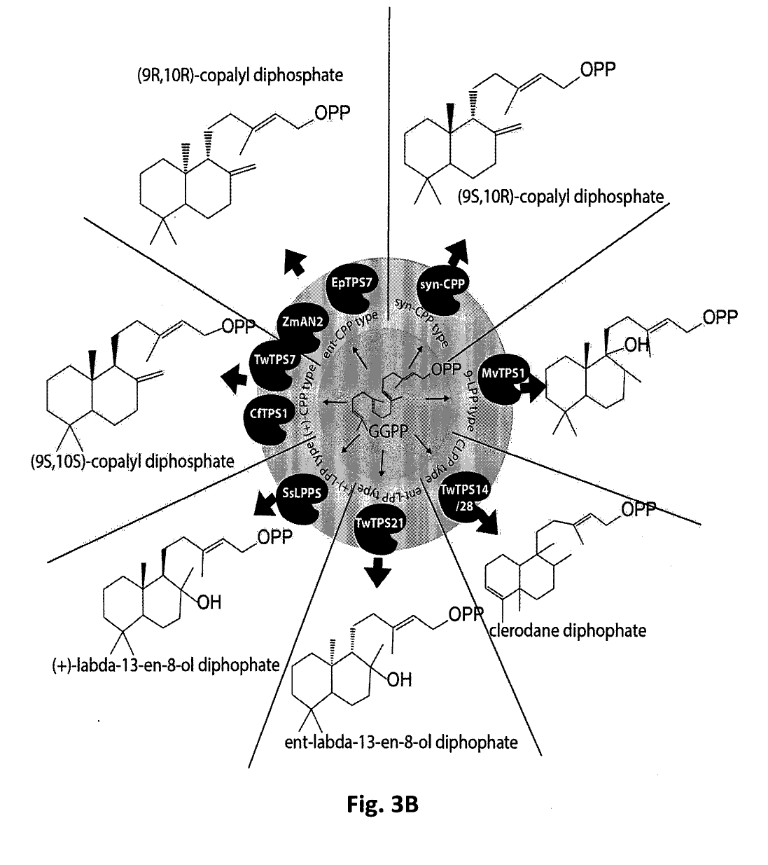

In preferred embodiments, the diterpene pyrophosphate intermediate may be any of the diterpene pyrophosphate intermediates shown in FIG. 3, i.e. the diterpene pyrophosphate intermediate may be selected from the group consisting of (9R,10R)-copalyl diphosphate, (9S,10S)-copalyl diphosphate, labda-13-en-8-ol diphosphate and (9S, 10R)-copalyl diphosphate.

Diterpenes

The term “diterpene” as used herein refers to a compound derived or prepared from four isoprene units. A diterpene according to the invention is a C₂₀-molecule consisting of 20 carbon atoms, up to three oxygen atoms and hydrogen atoms.

The diterpene typically contains one or more ring structures, such as one or more monocyclic, bicyclic, tricyclic or tetracyclic ring structure(s). The diterpene may contain one or more double bonds. Frequently, a diterpene according to the invention contains at least one double bond and often they contain in the range of 1 to 3 double bonds.

The diterpene may comprise up to three oxygen atom, although it is also possible that the diterpene contains no oxygen and consists solely of carbon and hydrogen atoms.

The oxygen atom are generally present in the form of hydroxyl groups, or part of a ring structure.

The term “diterpenoid” refers to a diterpene, which has been functionalised by addition of one or more functional groups.

In principle, the methods of the invention can be used to produce any diterpene by selecting an appropriate combination of diTPS of class II and diTPS of class I.

In one preferred embodiment the diterpene to be produce is a C₂₀-molecule containing a decalin core structure.

As used herein the term “containing a core structure of formula” or the term “containing a core of formula” refers to a molecule containing a structure of the indicated formula, wherein said structure may be substituted at one or more positions. The term “substituted” as used herein in relation to organic compounds refer to one hydrogen being substituted with another group or atom.

Said decalin may be substituted at one or more positions, and it is also contained within the invention that two substituents are fused, thus leading to a tricyclic or higher cyclic structure.

In particular, the diterpene to be produced by the methods of the present invention may be a C₂₀-molecule containing a core structure of one of following formulas XI, XII, XIII, XIV, XV, XVI, XVII, XVIII or XIX:

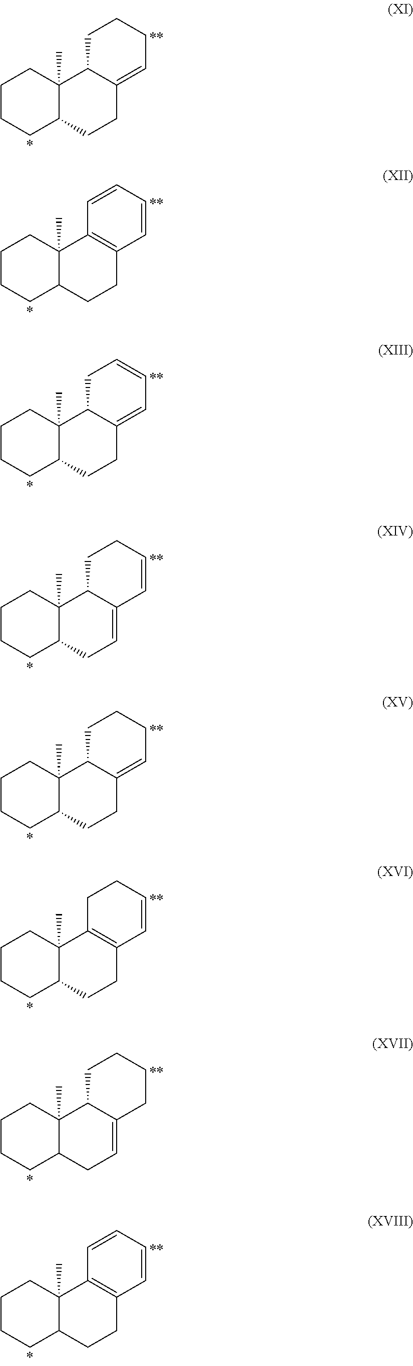

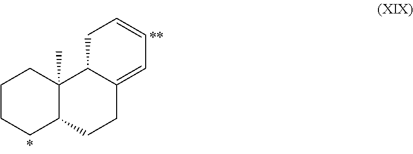

The diterpene containing a core structure of any of formulas XI, XII, XIII, XIV, XV, XVI, XVII, XVIII or XIX, may be a C₂₀-molecule consisting of the formulas XI, XII, XIII, XIV, XV, XVI, XVII, XVIII or XIX substituted at one or more positions. In particular, said diterpene may be a C₂₀-molecule substituted at the position marked by * with one or two alkyl, such as one or two C₁₋₃-alkyl, such as with one or two methyl groups. In addition said diterpene may be substituted at the position marked by ** with one or two groups individually selected from alkyl and alkenyl. Said alkyl may for example be C₁₋₆-alkyl, such as C₁₋₃-alkyl, for example isopropyl or methyl. Said alkenyl may me C₁₋₆ alkenyl, such as C₂₋₄-alkenyl, such as C₂₋₃-alkenyl.

In preferred embodiments of the invention the diterpene to be produced may be a C₂₀-molecule containing a core structure of one of following formulas I, II, III, IV, V, VI, IX or X:

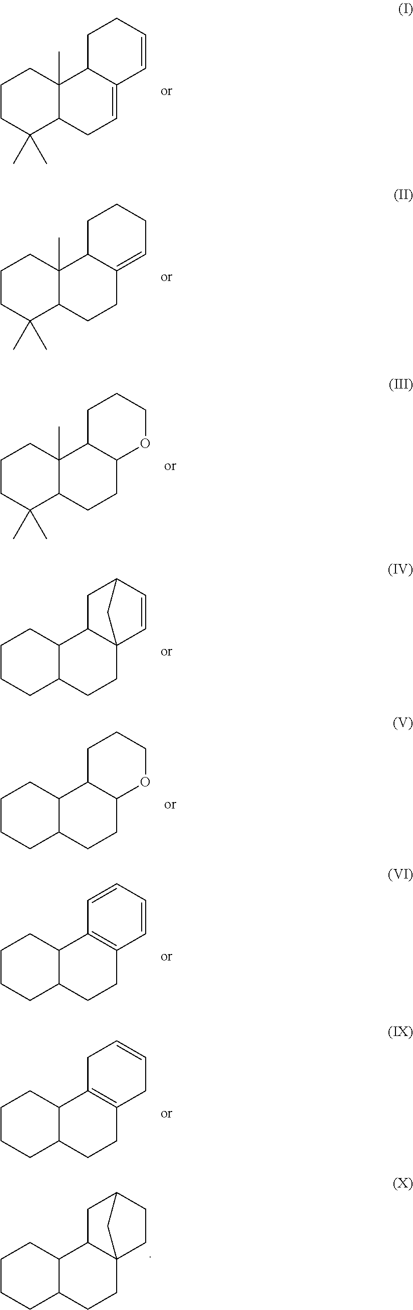

The diterpene containing a core structure of any of formulas I, II, III, IV, V, VI, IX or X, may be a C₂₀-molecule consisting of the formulas I, II, III, IV, V, VI, IX or X substituted at one or more positions, for example by one or more groups selected from the group consisting of:
c) alkyl, such as C₁₋₆-alkyl, for example C₁₋₃, wherein said alkyl may be linear or branched, for example alkyl may be isopropyl or methyl
d) alkenyl, such as C₁₋₆ alkenyl, such as C₂₋₄-alkenyl, such as C₂₋₃-alkenyl
e) hydroxyl

In particular said diterpene containing a core structure of any of formulas formulas I, II, III, IV, V, VI, IX or X, may be a C₂₀-molecule substituted
a) at the position corresponding to the 4 position of decalin with one or two alkyl, such as one or two C₁₋₃-alkyl, such as with one or two methyl groups, for example with two methyl; and/or
b) at the position corresponding to the 10 position of decalin with alkyl, such as with C₁₋₃-alkyl, such as with methyl; and/or
c) at the position corresponding to the position marked by ** in relations to formulas XI-XIX, with one or two groups individually selected from alkyl and alkenyl. Said alkyl may for example be C₁₋₆-alkyl, such as C₁₋₃-alkyl, for example isopropyl or methyl. Said alkenyl may me C₁₋₆ alkenyl, such as C₂₋₄-alkenyl, such as C₂₋₃-alkenyl; and/or
d) hydroxyl.

The diterpene to be produced may also be a C₂₀-molecule consisting of 20 carbon atoms, up to three oxygen atoms and hydrogen atoms, and which contains a core structure of any of formulas I, II, III, IV, VI, X, XXII, XXIII, XXIV, XXV, XXVI, XXVII, XXVIII, XXIX, XXX, XXXI, XXXII, XXXIII, XXXIV, XXXV, XXXVI, XXXVIII, XXXIX, XL and/or XLI.

The diterpene to be produced may also be a C₂₀-molecule consisting of 20 carbon atoms, up to three oxygen atoms and hydrogen atoms, and which contains a core structure of any of formulas I, II, IV, VI, X, XXII, XXIII, XXIV, XXVI, XXVII, XXVIII, XXIX, XXX, XXXI, XXXIII, XXXIV, XXXV, XXXVI, XXXVII, XXXVIII, XXXIX, XL and/or XLI.

The structure of the formulas I, II, III, IV, VI, X, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL and XLI are as indicated herein above.

In one embodiment the diterpene is a C₂₀-molecule containing a core of formula XXXIII:

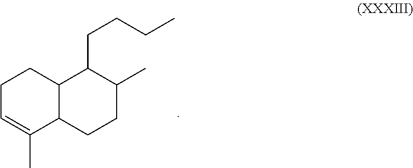 [SMILES: CCCCC1C(C)CCC2C(C)=CCCC21]

Said diterpene may in particular contain a core of formula XXXIII substituted with alkyl, alkenyl and/or hydroxyl, preferably substituted with methyl, ═CH₂ and hydroxyl.

In another embodiment the diterpene is a C₂₀-molecule containing a core of any of formulas II, XXXV, XXXVI and/or XXXVII:

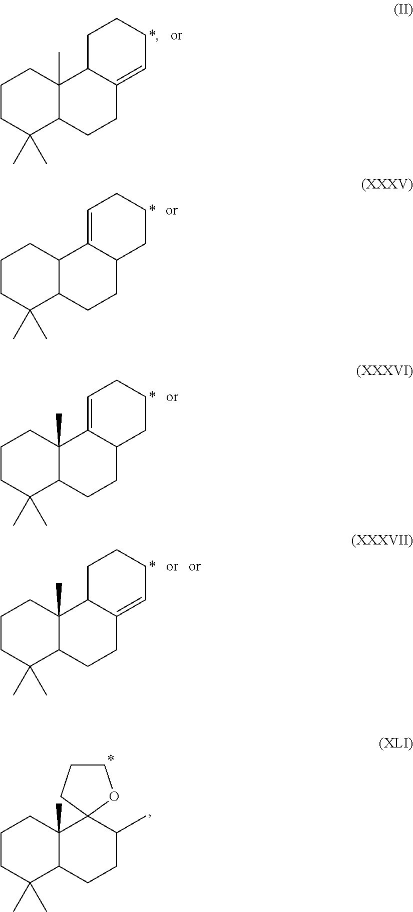

wherein said core may be substituted with one or more alkyl or alkenyl. In particular, the position marked by asterisk may be substituted with one or two substituents selected from the group consisting of C₁₋₂-alkyl and C₁₋₂-alkenyl, preferably the position marked by asterisk may be substituted with one methyl group and ethenyl group.

In one embodiment, said diterpene to be produced is a C₂₀-molecule containing a decalin substituted at the 10 position with C₅-alkenyl chain, which optionally may be substituted with a hydroxyl and/or a methyl group and/or ═C. For example, said diterpene may be a C₂₀-molecule of the formula XX:

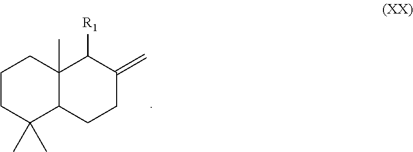

wherein R₁ is a C₅-alkenyl substituted with methyl and/or hydroxyl. Preferably, R₁ is C₅-alkenyl containing one or two double bonds. When R₁ is alkenyl containing one double bond, said alkenyl is preferably substituted with hydroxyl and methyl. When R₁ is alkenyl containing two double bonds, said alkenyl is preferably substituted with methyl.

For example, said diterpene may be a C₂₀-molecule of the formula XXI:

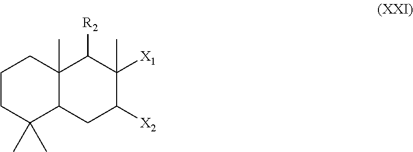

wherein R₂ is a C₅-alkenyl substituted with methyl and/or hydroxyl or with ═C, and X₁ is either —OH or methyl, and X₂ is either —H or —OH, wherein one and only one of X₁ and X₂ is —OH. Preferably, R₂ is C₅-alkenyl containing one or two double bonds. When R₂ is alkenyl containing one double bond, said alkenyl is preferably substituted with hydroxyl and methyl or with ═C. When R₂ is alkenyl containing two double bonds, said alkenyl is preferably substituted with methyl.

It is also comprised within the invention that the diterpene is the product of any of the reactions VII to XIX described herein above.

In particular, the diterpene may be any of the compounds 1 to 47 shown in FIG. 2 and/or Table 1.

It is preferred that the diterpene to be produced is not 13R-manoyl oxide.

Host Organism

The host organism to be used with the methods of the invention, may be any suitable host organism containing

a heterologous nucleic acid encoding a diTPS of class II, which may be any of diTPS of class II described herein in any of the sections “diTPS of class II”, “syn-CPP type diTPS”, “ent-CPP type diTPS”, “(+)-CPP type diTPS”, “LPP type diTPS”, and “LPP like type diTPS”; and
a heterologous nucleic acid encoding a diTPS of class I, which may be any of diTPS of class I described herein in any of the sections “diTPS of class I”, “EpTPS8”, “EpTPS23”, “SsSCS”, “CfTPS3”, “CfTPS4”, “MvTPS5”, “TwTPS2”, “EpTPS1”, and “CfTPS14”.

Suitable host organisms include microorganisms, plant cells, and plants.

The microorganism can be any microorganism suitable for expression of heterologous nucleic acids. In one embodiment the host organism of the invention is a eukaryotic cell. In another embodiment the host organism is a prokaryotic cell.

In a preferred embodiment, the host organism is a fungal cell such as a yeast or filamentous fungus. In particular the host organism may be a yeast cell.

In a further embodiment the yeast cell is selected from the group consisting of Saccharomyces cerevisiae, Schizosaccharomyces pombe, Yarrowia lipolytica, Candida glabrata, Ashbya gossypii, Cyberlindnera jadinii, and Candida albicans.

In general, yeasts and fungi are excellent microorganism to be used with the present invention. They offer a desired ease of genetic manipulation and rapid growth to high cell densities on inexpensive media. For instance yeasts grow on a wide range of carbon sources and are not restricted to glucose. Thus, the microorganism to be used with the present invention may be selected from the group of yeasts described below:

Arxula adeninivorans (Blastobotrys adeninivorans) is a dimorphic yeast (it grows as a budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Candida boidinii is a methylotrophic yeast (it can grow on methanol). Like other methylotrophic species such as Hansenula polymorpha and Pichia pastoris, it provides an excellent platform for the production of heterologous proteins. Yields in a multigram range of a secreted foreign protein have been reported. A computational method, IPRO, recently predicted mutations that experimentally switched the cofactor specificity of Candida boidinii xylose reductase from NADPH to NADH. Details on how to download the software implemented in Python and experimental testing of predictions are outlined in the following paper.

Hansenula polymorpha (Pichia angusta) is another methylotrophic yeast (see Candida boidinii). It can furthermore grow on a wide range of other substrates; it is thermo-tolerant and can assimilate nitrate (see also Kluyveromyces lactis). It has been applied to the production of hepatitis B vaccines, insulin and interferon alpha-2a for the treatment of hepatitis C, furthermore to a range of technical enzymes.

Kluyveromyces lactis is a yeast regularly applied to the production of kefir. It can grow on several sugars, most importantly on lactose which is present in milk and whey. It has successfully been applied among others to the production of chymosin (an enzyme that is usually present in the stomach of calves) for the production of cheese. Production takes place in fermenters on a 40,000 L scale.

Pichia pastoris is a methylotrophic yeast (see Candida boidinii and Hansenula polymorpha). It provides an efficient platform for the production of foreign proteins. Platform elements are available as a kit and it is worldwide used in academia for the production of proteins. Strains have been engineered that can produce complex human N-glycan (yeast glycans are similar but not identical to those found in humans).

Saccharomyces cerevisiae is the traditional baker's yeast known for its use in brewing and baking and for the production of alcohol. As protein factory it has successfully been applied to the production of technical enzymes and of pharmaceuticals like insulin and hepatitis B vaccines. Also it has been useful for production of terpenoids.

Yarrowia lipolytica is a dimorphic yeast (see Arxula adeninivorans) that can grow on a wide range of substrates. It has a high potential for industrial applications.

In another embodiment the host organism is a microalgae such as Chlorella and Prototheca.

In another embodiment of the invention the host organism is a filamentous fungus, for example Aspergillus.

In further yet another embodiment the host organism is a plant cell. The host organism may be a cell of a higher plant, but the host organism may also be cells from organisms not belonging to higher plants for example cells from the moss Physcomitrella patens.

In another embodiment the host organism is a mammalian cell, such as a human, feline, porcine, simian, canine, murine, rat, mouse or rabbit cell.

As mentioned, the host organism can also be a prokaryotic cell such as a bacterial cell. If the host organism is a prokaryotic cell the cell may be selected from, but not limited to E. coli, Corynebacterium, Bacillus, Pseudomonas and Streptomyces cells.

The host organism may also be a plant.

A plant or plant cell can be transformed by having a heterologous nucleic acid integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions. Both transiently transformed and stably transformed transgenic plants and plant cells can be useful in the methods described herein.

Plant cells comprising a heterologous nucleic acid used in methods described herein can constitute part or all of a whole plant. Such plants can be grown in a manner suitable for the species under consideration, either in a growth chamber, a greenhouse, or in a field. Plants may also be progeny of an initial plant comprising a heterologous nucleic acid provided the progeny inherits the heterologous nucleic acid. Seeds produced by a transgenic plant can be grown and then selfed (or outcrossed and selfed) to obtain seeds homozygous for the nucleic acid construct.

The plants to be used with the invention can be grown in suspension culture, or tissue or organ culture. For the purposes of this invention, solid and/or liquid tissue culture techniques can be used. When using solid medium, plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

The plant comprising a heterologous nucleic acid to be used with the present invention may for example be selected from: corn (Zea. mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (Helianthus annuas), wheat (Tritium aestivum and other species), Triticale, Rye (Secale) soybean (Glycine max), tobacco (Nicotiana tabacum or Nicothiana Benthamiana), potato (Solanum tuberosum), peanuts (Arachis hypogaea), cotton (Gossypium hirsutum), sweet potato (Impomoea batatus), cassava (Manihot esculenta), coffee (Cofea spp.), coconut (Cocos nucifera), pineapple (Anana comosus), citrus (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Primus amygdalus), apple (Malus spp), Pear (Pyrus spp), plum and cherry tree (Prunus spp), Ribes (currant etc.), Vitis, Jerusalem artichoke (Helianthemum spp), non-cereal grasses (Grass family), sugar and fodder beets (Beta vulgaris), chicory, oats, barley, vegetables, and ornamentals.

For example, plants of the present invention are crop plants (for example, cereals and pulses, maize, wheat, potatoes, tapioca, rice, sorghum, millet, cassava, barley, pea, sugar beets, sugar cane, soybean, oilseed rape, sunflower and other root, tuber or seed crops. Other important plants maybe fruit trees, crop trees, forest trees or plants grown for their use as spices or pharmaceutical products (Mentha spp, clove, Artemesia spp, Thymus spp, Lavendula spp, Allium spp., Hypericum, Catharanthus spp, Vinca spp, Papaver spp., Digitalis spp, Rawolfia spp., Vanilla spp., Petrusilium spp., Eucalyptus, tea tree, Picea spp, Pinus spp, Abies spp, Juniperus spp. Horticultural plants which may be used with the present invention may include lettuce, endive, and vegetable brassicas including cabbage, broccoli, and cauliflower, carrots, and carnations and geraniums.

The plant may also be selected from the group consisting of tobacco, cucurbits, carrot, strawberry, sunflower, tomato, pepper and Chrysanthemum.

The plant may also be a grain plants for example oil-seed plants or leguminous plants. Seeds of interest include grain seeds, such as corn, wheat, barley, sorghum, rye, etc. Oil-seed plants include cotton soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, chickpea.

In a further embodiment of the invention said plant is selected from the following group: maize, rice, wheat, sugar beet, sugar cane, tobacco, oil seed rape, potato and soybean. Thus, the plant may for example be rice.

The whole genome of Arabidopsis thaliana plant has been sequenced (The Arabidopsis Genome Initiative (2000). “Analysis of the genome sequence of the flowering plant Arabidopsis thaliana”. Nature 408 (6814): 796-815. doi:10.1038/35048692. PMID 11130711). Consequently, very detailed knowledge is available for this plant and it may therefore be a useful plant to work with. Accordingly, one plant, which may be used with the present invention is an Arabidopsis and in particular an Arabidopsis thaliana.

In one embodiment of the invention, the host organism may comprise at least the following heterologous nucleic acids:
a) a heterologous nucleic acid encoding Ossyn-CPP of SEQ ID NO:1 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas XXVI and/or XXVII, for example for production of compound 11 shown in FIG. 2.

In another embodiment of the invention, the host organism may comprise at least the following heterologous nucleic acids:
a) a heterologous nucleic acid encoding Ossyn-CPP of SEQ ID NO:1 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding MvTPS5 of SEQ ID NO:18 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas II, VI, XXXVIII, XXXV, or XXXVI, for example for production of compounds 6, 19 and/or 22 shown in FIG. 2B.

In another embodiment of the invention, the host organism may comprise at least the following heterologous nucleic acids:
a) a heterologous nucleic acid encoding Ossyn-CPP of SEQ ID NO:1 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding CfTPS4 of SEQ ID NO:13 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas II, VI, XXXVIII, XXXV, or XXXVI, for example for production of compounds 6, 19 and/or 22 shown in FIG. 2B.

In another embodiment of the invention, the host organism may comprise at least the following heterologous nucleic acids:
a) a heterologous nucleic acid encoding Ossyn-CPP of SEQ ID NO:1 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding CfTP3 of SEQ ID NO:12 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas II, VI, XXXVIII, XXXV, or XXXVI, for example for production of compounds 6, 19 and/or 22 shown in FIG. 2B.

In another embodiment of the invention, the host organism may comprise at least the following heterologous nucleic acids:
a) a heterologous nucleic acid encoding EpTPS7 of SEQ ID NO:2, ZmAN2 of SEQ ID NO:3 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas XXVI or XXVIII, for example for production of compound 23b shown in FIG. 2B.

In another embodiment of the invention, the host organism may comprise at least the following heterologous nucleic acids:
a) a heterologous nucleic acid encoding EpTPS7 of SEQ ID NO:2, ZmAN2 of SEQ ID NO:3 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding TwTPS2 of SEQ ID NO:14 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas IV or X, for example for production of compounds 15, 21 or 45 shown in FIG. 2B.

In another embodiment of the invention, the host organism may comprise at least the following heterologous nucleic acids:
a) a heterologous nucleic acid encoding EpTPS7 of SEQ ID NO:2, ZmAN2 of SEQ ID NO:3 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding EpTPS1 of SEQ ID NO:15 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formula X, for example for production of compound 21 shown in FIG. 2B.

In another embodiment of the invention, the host organism may comprise at least the following heterologous nucleic acids:
a) a heterologous nucleic acid encoding EpTPS7 of SEQ ID NO:2, ZmAN2 of SEQ ID NO:3 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding CfTPS14 of SEQ ID NO:16 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formula X, for example for production of compound 21 shown in FIG. 2B.

In another embodiment of the invention, the host organism may comprise at least the following heterologous nucleic acids:
a) a heterologous nucleic acid encoding EpTPS7 of SEQ ID NO:2, ZmAN2 of SEQ ID NO:3 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding EpTPS8 of SEQ ID NO:9 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas I, II, VI, XXII, XXIII or XXIV, for example for production of compounds 22, 27a/b or 34 shown in FIG. 2B.

In another embodiment of the invention, the host organism may comprise at least the following heterologous nucleic acids:
a) a heterologous nucleic acid encoding EpTPS7 of SEQ ID NO:2, ZmAN2 of SEQ ID NO:3 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding EpTPS23 of SEQ ID NO:10 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formula II or XXIV, for example for production of compound 9a/b shown in FIG. 2B.

In another embodiment of the invention, the host organism may comprise at least the following heterologous nucleic acids:
a) a heterologous nucleic acid encoding TwTPS7 of SEQ ID NO:4, CfTPS1 of SEQ ID NO:5 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding EpTPS8 of SEQ ID NO:9 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formula I, II, XXIII or XXIV, for example for production of compounds 9a/b or 27a/b shown in FIG. 2B.

In another embodiment of the invention, the host organism may comprise at least the following heterologous nucleic acids:
a) a heterologous nucleic acid encoding TwTPS7 of SEQ ID NO:4, CfTPS1 of SEQ ID NO:5 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding CfTPS4 of SEQ ID NO:13 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas VI, XXXIX or XL, for example for production of compounds 22 or 25 shown in FIG. 2B.

In another embodiment of the invention, the host organism may comprise at least the following heterologous nucleic acids:
a) a heterologous nucleic acid encoding TwTPS7 of SEQ ID NO:4, CfTPS1 of SEQ ID NO:5 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding CfTPS3 of SEQ ID NO:12 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas VI, XXXIX or XL, for example for production of compounds 22 or 25 shown in FIG. 2B.

In another embodiment of the invention, the host organism may comprise at least the following heterologous nucleic acids:
a) a heterologous nucleic acid encoding TwTPS7 of SEQ ID NO:4, CfTPS1 of SEQ ID NO:5 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding MvTPS5 of SEQ ID NO:18 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas VI, XXXIX or XL, for example for production of compounds 22 or 25 shown in FIG. 2B.

In another embodiment of the invention, the host organism may comprise at least the following heterologous nucleic acids:
a) a heterologous nucleic acid encoding TwTPS7 of SEQ ID NO:4, CfTPS1 of SEQ ID NO:5 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas XXVI or XXIX, for example for production of compound 23a shown in FIG. 2B.

In another embodiment of the invention, the host organism may comprise at least the following heterologous nucleic acids:
a) a heterologous nucleic acid encoding SsLPPS of SEQ ID NO:6, CfTPS2 of SEQ ID NO:17 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding MvTPS5 of SEQ ID NO:18 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III or XXV, for example for production of compound 16a shown in FIG. 2B.

In another embodiment of the invention, the host organism may comprise at least the following heterologous nucleic acids:
a) a heterologous nucleic acid encoding SsLPPS of SEQ ID NO:6, CfTPS2 of SEQ ID NO:17 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III, XXV, XXVI, XXX, XXXI, XXXII, XXXIII or XXXIV for example for production of compounds 3, 16a, 16b, 20, 23a/b, 26, 30, 36 or 43 shown in FIG. 2B.

In another embodiment of the invention, the host organism may comprise at least the following heterologous nucleic acids:
a) a heterologous nucleic acid encoding TwTPS21 of SEQ ID NO:7 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III, XXV, XXVI, XXX, XXXI, XXXII, XXXIII or XXXIV for example for production of compounds 3, 16a, 16b, 20, 23a/b, 26, 30, 36 or 43 shown in FIG. 2B.

In another embodiment of the invention, the host organism may comprise at least the following heterologous nucleic acids:
a) a heterologous nucleic acid encoding TwTPS21 of SEQ ID NO:7 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding CfTPS3 of SEQ ID NO:12 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III or XXXII for example for production of compound 16b shown in FIG. 2B.

In another embodiment of the invention, the host organism may comprise at least the following heterologous nucleic acids:
a) a heterologous nucleic acid encoding TwTPS21 of SEQ ID NO:7 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding TwTPS2 of SEQ ID NO:14 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III or XXXII for example for production of compound 20 shown in FIG. 2B.

In another embodiment of the invention, the host organism may comprise at least the following heterologous nucleic acids:
a) a heterologous nucleic acid encoding TwTPS21 of SEQ ID NO:7 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding CfTPS14 of SEQ ID NO:16 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III or XXXII for example for production of compound 20 shown in FIG. 2B.

In another embodiment of the invention, the host organism may comprise at least the following heterologous nucleic acids:
a) a heterologous nucleic acid encoding TwTPS21 of SEQ ID NO:7 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding EpTPS1 of SEQ ID NO:15 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formulas III or XXXII for example for production of compound 20 shown in FIG. 2B.

In another embodiment of the invention, the host organism may comprise at least the following heterologous nucleic acids:
a) a heterologous nucleic acid encoding TwTPS14/28 of SEQ ID NO:8 or a functional homologue thereof sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formula XXXIII, for example for production of compound 26 shown in FIG. 2B.

In another embodiment of the invention, the host organism may comprise at least the following heterologous nucleic acids:
a) a heterologous nucleic acid encoding TwTPS14/28 of SEQ ID NO:8 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding MvTPS5 of SEQ ID NO:18, CfTPS3 of SEQ ID NO:12, CfTPS4 of SEQ ID NO:13 or a functional homologue of any of the aforementioned sharing at least 70%, such as at least 80%, such as at least 85%, such as at least 90%, such as at least 95%, such as at least 98%, such as at least 99% sequence identity therewith.

In another embodiment of the invention, the host organism may comprise at least the following heterologous nucleic acids:
a) a heterologous nucleic acid encoding MvTPS1 of SEQ ID NO:28 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding SsSCS of SEQ ID NO:11 or a functional homologue thereof sharing at least 70%, such as at least 80%, such as at least 85%, such as at least 90%, such as at least 95%, such as at least 98%, such as at least 99% sequence identity therewith.

Such a host organism is in particular useful for production of diterpenes having a core of formula XLI, for example for production of compound 5 shown in FIG. 2B.

In another embodiment of the invention, the host organism may comprise at least the following heterologous nucleic acids:
a) a heterologous nucleic acid encoding MvTPS1 of SEQ ID NO:28 or a functional homologue of any of the aforementioned sharing at least 70%, such as at least 80%, such as at least 85%, such as at least 90%, such as at least 95%, such as at least 98%, such as at least 99% sequence identity therewith; and
b) a heterologous nucleic acid encoding CfTPS3 of SEQ ID NO:12, CfTPS4 of SEQ ID NO:13, EpTPS8 of SEQ ID NO:9, EpTPS23 of SEQ ID NO:10 or a functional homologue of any of the aforementioned sharing at least 70%, such as at least 80%, such as at least 85%, such as at least 90%, such as at least 95%, such as at least 98%, such as at least 99% sequence identity therewith.
Such a host organism is in particular useful for production of diterpenes having a core of formula XLI, for example for production of compound 5 shown in FIG. 2B.

It may be preferred that the host organism does not naturally produce the diterpene to be produced by the methods of the invention.

Sequences

Os syn-CPP
SEQ ID NO: 1
MPVFTASFQCVTLFGQPASAADAQPLLQGQRPFLHLHARRRRPCGPMLISKSPPYPASEE
TREWEAEGQHEHTDELRETTTTMIDGIRTALRSIGEGEISISAYDTSLVALLKRLDGGDG
PQFPSTIDWIVQNQLPDGSWGDASFFMMGDRIMSTLACVVALKSWNIHTDKCERGLLFIQ
ENMWRLAHEEEDWMLVGFEIALPSLLDMAKDLDLDIPYDEPALKAIYAERERKLAKIPRD
VLHAMPTTLLHSLEGMVDLDWEKLLKLRCLDGSFHCSPASTATAFQQTGDQKCFEYLDGI
VKKFNGGVPCIYPLDVYERLWAVDRLTRLGISRHFTSEIEDCLDYIFRNWTPDGLAHTKN
CPVKDIDDTAMGFRLLRLYGYQVDPCVLKKFEKDGKFFCLHGESNPSSVTPMYNTYRASQ
LKFPGDDGVLGRAEVFCRSFLQDRRGSNRMKDKWAIAKDIPGEVEYAMDYPWKASLPRIE
TRLYLDQYGGSGDVWIGKVLHRMTLFCNDLYLKAAKADFSNFQKECRVELNGLRRWYLRS
NLERFGGTDPQTTLMTSYFLASANIFEPNRAAERLGWARVALLADAVSSHFRRIGGPKNL
TSNLEELISLVPFDDAYSGSLREAWKQWLMAWTAKESSQESIEGDTAILLVRAIEIFGGR
HVLTGQRPDLWEYSQLEQLTSSICRKLYRRVLAQENGKSTEKVEEIDQQLDLEMQELTRR
VLQGCSAINRLTRETFLHVVKSFCYVAYSPETIDNHIDKVIFQDVI*
EpTPS7
SEQ ID NO: 2
MAAAANPSNSILNHHLLSSAAARSVSTSQLLFHSRPLVLSGAKDKRDSFVFRIKCSAVSN
PRIQEQTDVFQKNGLPVIKWHEFVETDIDHEQVSKVSVSNEIKKRVESIKAILESMEDGD
ITISAYDTAWVALVEDINGSGAPQFPASLQWIANNQLPDGSWGDAEIFTAHDRILNTLSC
VVALKSWNIHPDMCERGMKYFRENLCKLEDENIEHMPIGFEVAFPSLLELAKKLEIQVPE
DSPVLKDVYDSRNLKLKKIPKDIMHKVPTTLLHSLEGMPGLEWEKLLKLQSKDGSFLFSP
SSTAYALMQTKDQNCLEYLTKIVHKFNGGVPNVYPVDLFEHIWAVDRLQRLGISRYFQPQ
LKDSVDYVARYWEEDGICWARNSSVHDVDDTAMGFRVLRSFGHHVSADVFKHFKKGDTFF
CFAGQSTQAVTGMYNLLRASQLMFPGEKILEEAKQFSSAFLKVKQDANEVLDKWIITKDL
PGEVKYALDIPWYASLPRVESRFYIEQYGGSDDVWIGKTLYRMPIVNNDEYLKLAKLDYN
NCQAVHRSEWDNIQKWYEESDLAEFGVSRREILMAYYLAAASIFEPEKSRERIAWAKTSV
LLNTIQAYFHENNSTIHEKAAFVQLFKSGFAINARKLEGKTMEKLGRIIVGTLNDVSLDT
AMAYGKDISRDLRHAWDICLQKWEESGDMHQGEAQLIVNTINLTSDAWNFNDLSSHYHQF
FQLVNEICYKLRKYKKNKVNDKKKTTTPEIESHMQELVKLVLESSDDLDSNLKQIFLTVA
RSFYYPAVCDAGTINYHIARVLFERVY*
ZmAN2
SEQ ID NO: 3
MVLSSSCTTVPHLSSLAVVQLGPWSSRIKKKTDTVAVPAAAGRWRRALARAQHTSESAAV
AKGSSLTPIVRTDAESRRTRWPTDDDDAEPLVDEIRAMLTSMSDGDISVSAYDTAWVGLV
PRLDGGEGPQFPAAVRWIRNNQLPDGSWGDAALFSAYDRLINTLACVVTLTRWSLEPEMR
GRGLSFLGRNMWKLATEDEESMPIGFELAFPSLIELAKSLGVHDFPYDHQALQGIYSSRE
IKMKRIPKEVMHTVPTSILHSLEGMPGLDWAKLLKLQSSDGSFLFSPAATAYALMNTGDD
RCFSYIDRTVKKFNGGVPNVYPVDLFEHIWAVDRLERLGISRYFQKEIEQCMDYVNRHWT
EDGICWARNSDVKEVDDTAMAFRLLRLHGYSVSPDVFKNFEKDGEFFAFVGQSNQAVTGM
YNLNRASQ1SFPGEDVLHRAGAFSYEFLRRKEAEGALRDKWIISKDLPGEVVYTLDFPWY
GNLPRVEARDYLEQYGGGDDVWIGKTLYRMPLVNNDVYLELARMDFNHCQALHQLEWQGL
KRWYTENRLMDFGVAQEDALRAYFLAAASVYEPCRAAERLAWARAAILANAVSTHLRNSP
SFRERLEHSLRCRPSEETDGSWFNSSSGSDAVLVKAVLRLTDSLAREAQPIHGGDPEDII
HKLLRSAWAEWVREKADAADSVCNGSSAVEQEGSRMVHDKQTCLLLARMIEISAGRAAGE
AASEDGDRRIIQLTGSICDSLKQKMLVSQDPEKNEEMMSHVDDELKLRIREFVQYLLRLG
EKKTGSSETRQTFLSIVKSCYYAAHCPPHVVDRHISRVIFEPVSAAK*
TwTPS7
SEQ ID NO: 4
MHSLLMKKVIMYSSQTTHVFPSPLHCTIPKSSSFFLDAPVVRLHCLSGHGAKKKRLHFDI
QQGRNAISKTHTPEDLYAKQEYSVPEIVKDDDKEEEVVKIKEHVDIIKSMLSSMEDGEIS
ISAYDTAWVALIQDIHNNGAPQFPSSLLWIAENQLPDGSWGDSRVFLAFDRIINTLACVV
ALKSWNVHPDKCERGISFLKENISMLEKDDSEHMLVGFEFGFPVLLDMARRLGIDVPDDS
PFLQEIYVQRDLKLKRIPKDILHNAPTTLLHSLEA1PDLDWTKLLKLQCQDGSLLFSPSS
TAMAFINTKDENCLRYLNYVVQRFNGGAPTVYPYDLFEHNWAVDRLQRLGISRFFQPEIR
ECMSYVYRYWTKDGIFCTRNSRVHDVDDTAMGFRLLRLHGYEVHPDAFRQFKKGCEFICY
EGQSHPTVTVMYNLYRASQLMFPEEKILDEAKQFTEKFLGEKRSANKLLDKWIITKDLPG
EVGFALDVPWYASLPRVEARFFIQHYGGEDDVWLDKALYRMPYVNNNVYLELAKLDYNYC
QALHRTEWGHIQKWYEECKPRDFGISRECLLRAYFMAAASIFEPERSMERLAWAKTAILL
ElIVSYFNEVGNSTEQRIAFTTEFSIRASPMGGYINGRKLDKIGTTQELIQMLLATIDQF
SQDAFAAYGHDITRHLHNSWKMWLLKWQEEGDRWLGEAELLIQTINLMADHKIAEKLFMG
HTNYEQLFSLTNKVCYSLGHHELQNNKELEHDMQRLVQLVLTNSSDGIDSDIKKTFLAVA
KRFYYTAFVDPETVNVHIAKVLFERVD*
CfTPS1
SEQ ID NO: 5
MGSLSTMNLNHSPMSYSGILPSSSAKAKLLLPGCFSISAWMNNGKNLNCQLTHKKISKVA
EIRVATVNAPPVHDQDDSTENQCHDAVNNIEDPIEYIRTLLRTTGDGRISVSPYDTAWVA
LIKDLQGRDAPEFPSSLEWIIQNQLADGSWGDAKFFCVYDRLVNTIACVVALRSWDVHAE
KVERGVRYINENVEKLRDGNEEHMTCGFEVVFPALLQRAKSLGIQDLPYDAPVIQEIYHS
REQKSKRIPLEMMHKVPTSLLFSLEGLENLEWDKLLKLQSADGSFLTSPSSTAFAFMQTR
DPKCYQFIKNTIQTFNGGAPHTYPVDVFGRLWAIDRLQRLGISRFFESEIADCIAHIHRF
WTEKGVFSGRESEFCDIDDTSMGVRLMRMHGYDVDPNVLKNFKKDDKFSCYGGQMIESPS
PlYNLYRASQLRFPGEQILEDANKFAYDFLQEKLAHNQILDKWVISKHLPDEIKLGLEMP
WYATLPRVEARYYIQYYAGSGDVWIGKTLYRMPEISNDTYHELAKTDFKRCQAQHQFEW1
YMQEWYESCNMEEFGISRKELLVAYFLATASIFELERANERIAWAKSQIISTIIASFFNN
QNTSPEDKLAFLTDFKNGNSTNMALVTLTQFLEGFDRYTSHQLKNAWSVWLRKLQQGEGN
GGADAELLVNTLNICAGHIAFREElLAHNDYKTLSNLTSKICRQLSQIQNEKELETEGQK
TSIKNKELEEDMQRLVKLVLEKSRVGINRDMKKTFLAVVKTYYYKAYHSAQAIDNHMFKV
LFEPVA*
SsLPPS
SEQ ID NO: 6
MTSVNLSRAPAAITRRRLQLQPEFHAECSWLKSSSKHAPLTLSCQIRPKQLSQIAELRVT
SLDASQASEKDISLVQTPHKVEVNEKIEESIEYVQNLLMTSGDGRISVSPYDTAVIALIK
DLKGRDAPQFPSCLEWIAHHQLADGSWGDEFFCIYDRILNTLACVVALKSWNLHSDIIEK
GVTYIKENVHKLKGANVEHRTAGFELVVPTFMQMATDLGIQDLPYDHPLIKEIADTKQQR
LKEIPKDLVYQMPTNLLYSLEGLGDLEWERLLKLQSGNGSFLTSPSSTAAVLMHTKDEKC
LKYIENALKNCDGGAPHTYPVDIFSRLWAIDRLQRLGISRFFQHEIKYFLDHIESVWEET
GVFSGRYTKFSDIDDTSMGVRLLKMHGYDVDPNVLKHFKQQDGKFSCYIGQSVESASPMY
NLYRAAQLRFPGEEVLEEATKFAFNFLQEMLVKDRLQERWVISDHLFDEIKLGLKMPWYA
TLPRVEAAYYLDHYAGSGDVWIGKSFYRMPEISNDTYKELAILDFNRCQTQHQLEWIHMQ
EWYDRCSLSEFGISKRELLRSYFLAAATIFEPERTQERLLWAKTRILSKMITSFVNISGT
TLSLDYNENGLDElISSANEDUGLAGTLLATFHQLLDGFDIYTLHQLKHVWSQWFMKVQQ
GEGSGGEDAVLLANTLNICAGLNEDVLSNNEYTALSTLTNKICNRLAQIQDNKILQVVDG
SIKDKELEQDMQALVKLVLQENGGAVDRNIRHTFLSVSKTFYYDAYHDDETTDLHIFKVL
FRPVV*
TwTPS21
SEQ ID NO: 7
MFMSSSSSSHARRPQLSSFSYLHPPLPFPGLSFFNTRDKRVNFDSTRIICIAKSKPARTT
PEYSDVLQTGLPLIVEDDIQEQEEPLEVSLENQIRQGVDIVKSMLGSMEDGETSISAYDT
AWVALVENIHHPGSPQFPSSLQWIANNQLPDGSWGDPDVFLAHDRLINTLACVIALKKWN
IHPHKCKRGLSFVKENISKLEKENEEHMLIGFEIAFPSLLEMAKKLGIEIPDDSPALQDI
YTKRDLKLTRIPKDKMHNVPTTLLHSLEGLPDLDWEKLVKLQFQNGSFLFSPSSTAFAFM
HTKDGNCLSYLNDLVHKFNGGVPTAYPVDLFEHIWSVDRLQRLGISRFFHPEIKECLGYV
HRYWTKDGICWARNSRVQDIDDTAMGFRLLRLHGYEVSPDVFKQFRKGDEFVCFMGQSNQ
AITGIYNLYRASQMMFPEETILEEAKKFSVNFLREKRAASELLDKWIITKDLPNEVGFAL
DVPWYACLPRVETRLYIEQYGGQDDVWIGKTLYRMPYVNNNVYLELAKLDYNNCQSLHRI
EWDNIQKWYEGYNLGGFGVNKRSLLRTYFLATSNIFEPERSVERLTWAKTAILVQAIASY
FENSREERIEFANEFQKFPNTRGYINGRRLDVKQATKGLIEMVFATLNQFSLDALVVHGE
DITHHLYQSWEKWVLTWQEGGDRREGEAELLVQTINLMAGHTHSQEEELYERLFKLTNTV
CHQLGHYHHLNKDKQPQQVEDNGGYNNSNPESISKLQIESDMRELVQLVLNSSDGMDSNI
KQTFLAVTKSFYYTAFTHPGTVNYHIAKVLFERVV*
TwTPS14/28
SEQ ID NO: 8
MFMSSSSSSHARRPQLSSFSYLHPPLPFPGLSFFNTRDKRVNFDSTRIICIAKSKPARTT
PEYSDVLQTGLPLIVEDDIQEQEEPLEVSLENQIRQGVDIVKSMLGSMEDGETSISAYDT
AWVALVENIHHPGSPQFPSSLQWIANNQLPDGSWGDPDVFLAHDRLINTLACVIALKKWN
IHPHKCKRGLSFVKENISKLEKENEEHMLIGFEIAFPSLLEMAKKLGIEIPDDSPALQDI
YTKRDLKLTRIPKDIMHNVPTTLLYSLEGLPSLDWEKLVKLQCTDGSFLFSPSSTACALM
HTKDGNCFSYINNLVHKFNGGVPTVYPVDLFEHIWCVDRLQRLGISRFFHPEIKECLGYV
HRYWTKDGICWARNSRVQDIDDTAMGFRLLRLHGYEVSPDVFKQFRKGDEFVCFMGQSNQ
AITGIYNLYRASQMMFPEETILEEAKKFSVNFLREKRAASELLDKWIITKDLPNEVGFAL
DVPWYACLPRVETRLYIEQYGGQDDVWIGKTLYRMPYVNNNVYLELAKLDYNNCQSLHRI
EWDNIQKWYEGYNLGGFGVNKRSLLRTYFLATSNIFEPERSVERLTWAKTAILVQAIASY
FENSREERIEFANEFQKFPNTRGYINGRRLDVKQATKGLIEMVFATLNQFSLDALVVHGE
DITHHLYQSWEKWVLTWQEGGDRREGEAELLVQTINLMAGHTHSQEEELYERLFKLTNTV
CHQLGHYHHLNKDKQPQQVEDNGGYNNSNPESISKLQIESDMRELVQLVLNSSDGMDSNI
KQTFLAVTKSFYYTAFTHPGTVNYHIAKVLFERVV*
EpTPS8
SEQ ID NO: 9
MQVSLSLTTGSEPCITRIHAPSDAPLKQRNNEREKGTLELNGKVSLKKMGEMLRTIENVP
IVGSTSSYDTAWVGMVPCSSNSSKPLFPESLKWIMENQNPEGNWAVDHAHHPLLLKDSLS
STLACVLALHKWNLAPQLVHSGLDFIGSNLWAAMDFRQRSPLGFDVIFPGMIHQAIDLGI
NLPFNNSSIENMLTNPLLDIQSFEAGKTSHIAYFAEGLGSRLKDWEQLLQYQTSNGSLFN
SPSTTAAAAIHLRDEKCLNYLHSLTKQFDNGAVPTLYPLDARTRISIIDSLEKFGIHSHF
IQEMTILLDQIYSFWKEGNEEIFKDPGCCATAFRLLRKHGYDVSSDSLAEFEKKEIFYHS
SAASAHEIDTKSILELFRASQMKILQNEPILDRIYDWTSIFLRDQLVKGLIENKSLYEEV
NFALGHPFANLDRLEARSYIDNYDPYDVPLLKTSYRSSNIDNKDLWTIAFQDFNKCQALH
RVELDYLEKWVKEYKLDTLKWARQKTEYALFTIGAILSEPEYADARISWSQNTVFVTIVD
DFFDYGGSLDECRNLINLMHKWDDHLTVGFLSEKVEIVFYSMYGTLNDLAAKAEVRQGRC
VRSHLVNLWIWVMENMLKEREWADYNLVPTFYEYVAAGHITIGLGPVLLIALYFMGYPLS
EDVVQSQEYKGVYLNVSIIARLLNDRVTVKRESAQGKLNGVSLFVEHGRGAVDEETSMKE
VERLVESHKRELLRLIVQKTEGSVVPQSCKDLAWRVSKVLHLLYMDDDGFTCPVKMLNAT
NAIVNEPLLLTS*
EpTPS23
SEQ ID NO: 10
MLLASSTSSRFFTKEWEPSNKTFSGSVRAQLSQRVKNIVVTPDQVKESESSGTSLRLKEM
LKKVEMPISSYDTAWVAMVPSMEHSRNKPLFPNSLKWVMENQQPDGSWCFDDSNHPWLIK
DSLSSTLASVLALKKWNVGQQLIDKGLEYIGSNMWAATDMHQYSPIGFNIIFPSMVEHAN
KLGLSLSLDHSLFQSMLRNRDMETKSLNGRNMAYVAEGLNGSNNWKEVMKYQRRNGSILN
SPATTAAALIHLNDVKCFEYLDSLLTKFQHAVPTLYPFDIYARLCILDELEKLGVDRFVE
IEKMIILDYIYRCWLEGSEEILEDPTCCAMAFRFLRMNGYVVSPDVLQGFEEEEKLFHVK
DTKSVLELLKASQLKVSEKEGILDRIYSWATSYLKHQLFNASISDKSLQNEVDYVVKHPH
AILRRIENRNYIENYNTKNVSLRKTSFRFVNVDKRSDLLAHSRQDFNKCQIQFKKELAYL
SRWEKKYGLDKLKYARQRLEVVYFSIASNLFEPEFSDARLAWTQYAILTTVVDDFFEYAA
SMDELVNLTNLIERWDEHGSEEFKSKEVEILFYAIYDLVNEDAEKAKKYQGRCIKSHLVH
IWIDILKAMLKESEYVRYNIVPTLDEYISNGCTSISFGAILLIPLYFLGKMSEEVVTSKE
YQKLYMHISMLGRLLNDRVTSQKDMAQGKLNSVSLRVLHSNGTLTEEEAKEEVDKIIEKH
RRELLRMVVQTEGSVVPKACKKLFWMTSKELHLFYMTEDCFTCPTKLLSAVNSTLKDPLL
MP*
SsSCS
SEQ ID NO: 11
MSLAFNVGVTPFSGQRVGSRKEKFPVQGFPVTTPNRSRLIVNCSLTTIDFMAKMKENFKR
EDDKFPTTTTLRSEDIPSNLCIIDTLQRLGVDQFFQYEINTILDNTFRLWQEKHKVIYGN
VTTHAMAFRLLRVKGYEVSSEELAPYGNQEAVSQQTNDLPMIIELYRAANERIYEEERSL
EKILAWTTIFLNKQVQDNSIPDKKLHKLVEFYLRNYKGITIRLGARRNLELYDMTYYQAL
KSTNRFSNLCNEDFLVFAKQDFDIHEAQNQKGLQQLQRWYADCRLDTLNFGRDVVIIANY
LASLIIGDHAFDYVRLAFAKTSVLVTIMDDFFDCHGSSQECDKIIELVKEWKENPDAEYG
SEELEILFMALYNTVNELAERARVEQGRSVKEFLVKLWVEILSAFKIELDTWSNGTQQSF
DEYISSSWLSNGSRLTGLLTMQFVGVKLSDEMLMSEECTDLARHVCMVGRLLNDVCSSER
EREENIAGKSYSILLATEKDGRKVSEDEAIAEINEMVEYHWRKVLQIVYKKESILPRRCK
DVFLEMAKGTFYAYGINDELTSPQQSKEDMKSFVF*
CfTPS3
SEQ ID NO: 12
MSSLAGNLRVIPFSGNRVQTRTGILPVHQTPMITSKSSAAVKCSLTTPTDLMGKIKEVFN
REVDTSPAAMTTHSTDIPSNLCIIDTLQRLGIDQYFQSEIDAVLHDTYRLWQLKKKDIFS
DITTHAMAFRIIRVKGYEVASDELAPYADQERINLQTIDVPTVVELYRAAQERLTEEDST
LEKLYVWTSAFLKQQLLTDAIPDKKLHKQVEYYLKNYHGILDRMGVRRNLDLYDISHYKS
LKAAHRFYNLSNEDILAFARQDFNISQAQHQKELQQLQRWYADCRLDTLKFGRDVVRIGN
FLTSAMIGDPELSDLRLAFAKHIVLVTRIDDFFDHGGPKEESYEILELVKEWKEKPAGEY
VSEEVEILFTAVYNTVNELAEMAHIEQGRSVKDLLVKLWVEILSVFRIELDTWTNDTALT
LEEYLSQSWVSIGCRICILISMQFQGVKLSDEMLQSEECTDLCRYVSMVDRLLNDVQTFE
KERKENTGNSVSLLQAAHKDERVINEEEACIKVKELAEYNRRKLMQIVYKTGTIFPRKCK
DLFLKACRIGCYLYSSGDEFTSPQQMMEDMKSLVYEPLPISPPEANNASGEKMSCVSN*
CfTPS4
SEQ ID NO: 13
MSITINLRVIAFPGHGVQSRQGIFAVMEFPRNKNTFKSSFAVKCSLSTPTDLMGKIKEKL
SEKVDNSVAAMATDSADMPTNLCIVDSLQRLGVEKYFQSEIDTVLDDAYRLWQLKQKDIF
SDITTHAMAFRLLRVKGYDVSSEELAPYADQEGMNLQTIDLAAVIELYRAAQERVAEEDS
TLEKLYVWTSTFLKQQLLAGAIPDQKLHKQVEYYLKNYHGILDRMGVRKGLDLYDAGYYK
ALKAADRLVDLCNEDLLAFARQDFNINQAQHRKELEQLQRWYADCRLDKLEFGRDVVRVS
NFLTSAILGDPELSEVRLVFAKHIVLVTRIDDFFDHGGPREESHKILELIKEWKEKPAGE
YVSKEVEILYTAVYNTVNELAERANVEQGRNVEPFLRTLWVQILSIFKIELDTWSDDTAL
TLDDYLNNSWVSIGCRICILMSMQFIGMKLPEEMLLSEECVDLCRHVSMVDRIINDVQTF
EKERKENTGNAVSLLLAAHKGERAFSEEEAIAKAKYLADCNRRSLMQIVYKTGTIFPRKC
KDMFLKVCRIGCYLYASGDEFTSPQQMMEDMKSLVYEPLQIHPPAAA*
TwTPS2
SEQ ID NO: 14
MFDKTQLSVSAYDTAWVAMVSSPNSRQAPWFPECVNWLLDNQLSDGSWGLPPHHPSLVKD
ALSSTLACLLALKRWGLGEQQMTKGLQFIESNFTSINDEEQHTPIGFNIIFPGMIETAID
MNLNLPLRSEDINVMLHNRDLELRRNKLEGREAYLAYVSEGMGKLQDWEMVMKYQRKNGS
LFNSPSTTAAALSHLGNAGCFHYINSLVAKFGNAVPTVYPSDKYALLCMIESLERLGIDR
HFSKEIRDVLEETYRCWLQGDEEIFSDADTCAMAFRILRVHGYEVSSDPLTQCAEHHFSR
SFGGHLKDFSTALELFKASQFV1FPEESGLEKQMSWTNQFLKQEFSNGTTRADRFSKYFS
IEVHDTLKFPFHANVERLAHRRNIEHHHVDNTRILKTSYCFSNISNADFLQLAVEDFNRC
QSIHREELKHLERWVVETKLDRLKFARQKMAYCYFSAAGTCFSPELSDARISWAKNSVLT
TVADDFFDIVGSEEELANLVHLLENWDANGSPHYCSEPVEIIFSALRSTICEIGDKALAW
QGRSVTHHVIEMWLDLLKSALREAEWARNKVVPTFDEYVENGYVSMALGPIVLPAVYLIG
PKVSEEVVRSPEFHNLFKLMSICGRLINDTRTFKRESEAGKLNSVLLHMIHSGSGTTEEE
AVEKIRGMIADGRRELLRLVLQEKDSVVPRACKDLFWKMVQVLHLFYMDGDGFSSPDMML
NAVNALIREPISL*
EpTPS1
SEQ ID NO: 15
MSATPNSFFTSPISAKLGHPKSQSVAESNTRIQQLDGTREKIKKMFDKVELSVSPYDTAW
VAMVPSPNSLEAPYFPECSKWIVDNQLNDGSWGVYHRDPLLVKDSISSTLACVLALKRWG
IGEKQVNKGLEFIELNSASLNDLKQYKPVGFDITFPRMLEHAKDFGLNLPLDPKYVEAVI
FSRDLDLKSGCDSTTEGRKAYLAYISEGIGNLQDWNMVMKYQRRNGSIFDSPSATAAASI
HLHDASCLRYLRCALKKFGNAVPTIYPFNIYVRLSMVDAIESLGIARHFQEEIKTVLDET
YRYWLQGNEEIFQDCTTCAMAFRILRANGYNVSSEKLNQFTEDHFSNSLGGYLEDMRPVL
ELYKASQLIFPDELFLEKQFSWTSQCLKQKISSGLRHTDGINKHITEEVNDVLKFASYAD
LERLTNWRRIAVYRANETKMLKTSYRCSNIANEHFLELAVEDFNVCQSMHREELKHLGRW
VVEKRLDKLKFARQKLGYCYFSSAASLFAPEMSDARISWAKNAVLTTVVDDFFDVGGSEE
ELINLVQLIERWDVDGSSHFCSEHVEIVFSALHSTICEIGEKAFAYQGRRMTSHVIKIWL
DLLKSMLTETLWSKSKATPTLNEYMTNGNTSFALGPIVLPALFFVGPKLTDEDLKSHELH
DLFKTMSTCGRLLNDWRSYERESEEGKLNAVSLHMIYGNGSVAATEEEATQKIKGLIESE
RRELMRLVLQEKDSKIPRPCKDLFWKMLKVLHMFYLKDDGFTSNQMMKTANSLINQPISL
HER*
CfTPS14
SEQ ID NO: 16
MSLPLSTCVLFVPKGSQFWSSRFSYASASLEVGFQRATSAQIAPLSKSFEETKGRIAKLF
HKDELSISTYDTAWVAMVPSPTSSEEPCFPACLNWLLENQCLDGSWARPHHHPMLKKDVL
SSTLACILALKKWGVGEEQINRGLHFlELNFASATEKCQITPMGFDIVFPAMLDRARALS
LNIRLEPTTLNDLMNKRDLELNRCYQSSSTEREVYRAYIAEGMGKLQNWESVMKYQRKNG
TLFNCPSTTAAAFTALRNSDCLNYLHLALNKFGDAVPAVFPLDIYSQLCIVDNLERVGIS
RHFLTEIQSVLDGTYRSWLQGDEQIFMDASTCALAFRTLRMNGYNVSSDPITKLIQEGSF
SRNTMDINTTLELYRASELILYPDERDLEEHNLRLKTILDQELSGGGFILSRQLGRNINA
EVKQALESPFYAIMDRMAKRRSIEHYHIDNTRILKTSYCSPNFGNEDFLSLSVEDFNRCQ
VIHREELRELERWVIENRLDELKFARSKSAYCYFSAAATIFSPELSDARMSWAKNGVLTT
VVDDFFDVGGSVEELKNLIQLVELWDVDVSRECISPSVQIIFSALKHTIREIGDKGFKLQ
GRSITDHIIAIWLDLLYSMMKESEWGREKAVPTIDEYISNAYVSFALGPIVLPALYLVGP
KLSEEMVNHADYHNLFKSMSTCGRLLNDIRGYERELKDGKLNTLSLYMVNNEGEISWEAA
ILEVKSWIERERRELLRSVLEEEKSVVPKACKELFWHMCTVVHLFYSKDDGFTSQDLLSA
VNAIIYQPLVLE*
CfTPS2
SEQ ID NO: 17
MKMLMIKSQFRVHSIVSAWANNSNKRQSLGHQIRRKQRSQVTECRVASLDALNGIQKVGP
ATIGTPEEENKKIEDSIEYVKELLKTMGDGRISVSPYDTAIVALIKDLEGGDGPEFPSCL
EWIAQNQLADGSWGDHFFCIYDRVVNTAACVVALKSWNVHADKIEKGAVYLKENVHKLKD
GKIEHMPAGFEFVVPATLERAKALGIKGLPYDDPFIREIYSAKQTRLTKIPKGMIYESPT
SLLYSLDGLEGLEWDKILKLQSADGSFITSVSSTAFVFMHTNDLKCHAFIKNALTNCNGG
VPHTYPVDIFARLWAVDRLQRLGISRFFEPEIKYLMDHINNVWREKGVFSSRHSQFADID
DTSMGIRLLKMHGYNVNPNALEHFKQKDGKFTCYADQHIESPSPMYNLYRAAQLRFPGEE
ILQQALQFAYNFLHENLASNHFQEKWVISDHLIDEVRIGLKMPWYATLPRVEASYYLQHY
GGSSDVWIGKTLYRMPEISNDTYKILAQLDFNKCQAQHQLEWMSMKEWYQSNNVKEFGIS
KKELLLAYFLAAATMFEPERTQERIMWAKTQVVSRMITSFLNKENTMSFDLKIALLTQPQ
HQINGSEMKNGLAQTLPAAFRQLLKEFDKYTRHQLRNTWNKWLMKLKQGDDNGGADAELL
ANTLNICAGHNEDILSHYEYTALSSLTNKICQRLSQIQDKKMLEIEEGSIKDKEMELEIQ
TLVKLVLQETSGGIDRNIKQTFLSVFKTFYYRAYHDAKTIDAHIFQVLFEPVV*
MvTPS5
SEQ ID NO: 18
MSITFNLKIAPFSGPGIQRSKETFPATEIQITASTKSTMTTKCSFNASTDFMGKLREKVG
GKADKPPVVIHPVDISSNLCMIDTLQSLGVDRYFQSEINTLLEHTYRLWKEKKKNIIFKD
VSCCAIAFRLLREKGYQVSSDKLAPFADYRIRDVATILELYRASQARLYEDEHTLEKLHD
WSSNLLKQHLLNGSIPDHKLHKQVEYFLKNYHGILDRVAVRRSLDLYNINHHHRIPDVAD
GFPKEDFLEYSMQDFNICQAQQQEELHQLQRWYADCRLDTLNYGRDVVRIANFLTSAIFG
EPEFSDARLAFAKHIILVTRIDDFFDHGGSREESYKILDLVQEWKEKPAEEYGSKEVEIL
FTAVYNTVNDLAEKAHIEQGRCVKPLLIKLWVEILTSFKKELDSWTEETALTLDEYLSSS
WVSIGCRICILNSLQYLGIKLSEEMLSSQECTDLCRHVSSVDRLLNDVQTFKKERLENTI
NSVGLQLAAHKGERAMTEEDAMSKIKEMADYHRRKLMQIVYKEGTVFPRECKDVFLRVCR
IGYYLYSSGDEFTSPQQMKEDMKSLVYQPVKIHPLEAINV*
codon optimized DNA sequence encoding truncated CfTPS1:
SEQ ID NO: 19
ATGGGTTCCTTGTCTACCATGAACTTGAACCATTCTCCAATGTCCTACTCTGGTATTTTG
CCATCTTCTTCAGCTAAGGCTAAGTTGTTGTTGCCAGGTTGTTTTTCTATTTCCGCTTGG
ATGAACAACGGTAAGAATTTGAATTGCCAATTGACCCACAAGAAGATCTCTAAGGTTGCC
GAAATTAGAGTTGCTACTGTTAATGCTCCACCAGTTCATGATCAAGATGACTCTACTGAA
AATCAATGCCATGATGCCGTTAACAACATCGAAGATCCAATCGAATATATCAGAACCTTG
TTGAGAACTACCGGTGATGGTAGAATTTCTGTTTCTCCATATGATACTGCTTGGGTCGCT
TTGATTAAGGACTTGCAAGGTAGAGATGCTCCAGAATTTCCATCTTCATTGGAATGGATC
ATCCAAAATCAATTGGCTGATGGTTCTTGGGGTGATGCTAAGTTTTTTTGCGTTTACGAT
AGATTGGTCAACACCATTGCTTGTGTTGTTGCTTTGAGATCTTGGGATGTTCATGCTGAA
AAAGTTGAAAGAGGTGTCAGATATATCAACGAAAACGTCGAAAAGTTGAGAGATGGTAAC
GAAGAACATATGACCTGTGGTTTCGAAGTTGTTTTCCCAGCTTTGTTGCAAAGAGCTAAG
TCTTTGGGTATTCAAGATTTGCCATATGATGCCCCAGTTATCCAAGAAATCTATCACTCT
AGAGAACAAAAGTCCAAGAGAATCCCATTGGAAATGATGCATAAGGTCCCAACTAGTTTG
TTGTTCTCTTTGGAAGGTTTGGAAAACTTGGAATGGGACAAGTTGTTGAAGTTGCAATCA
GCAGATGGTTCCTTTTTGACTTCTCCATCTTCTACTGCTTTCGCTTTCATGCAAACTAGA
GATCCAAAGTGCTACCAATTCATCAAGAACACCATTCAAACTTTCAACGGTGGTGCTCCA
CATACTTATCCAGTTGATGTTTTTGGTAGATTGTGGGCCATTGACAGATTGCAAAGATTG
GGTATTTCCAGATTCTTCGAATCCGAAATTGCTGACTGCATTGCCCATATTCATAGATTC
TGGACTGAAAAGGGTGTTTTCTCTGGTAGAGAATCTGAATTCTGCGATATCGATGATACC
TCTATGGGTGTTAGATTGATGAGAATGCATGGTTACGATGTTGATCCAAACGTCTTGAAG
AATTTCAAGAAGGACGATAAGTTCTCTTGCTACGGTGGTCAAATGATTGAATCTCCATCT
CCAATCTACAACTTGTACAGAGCTTCCCAATTGAGATTTCCAGGTGAACAAATTTTGGAA
GATGCCAACAAGTTCGCCTACGACTTTTTACAAGAAAAGTTGGCCCATAATCAAATCTTG
GACAAGTGGGTTATTTCCAAACATTTGCCAGACGAAATCAAGTTGGGTTTAGAAATGCCA
TGGTATGCTACTTTGCCAAGAGTTGAAGCCAGATATTACATCCAATATTACGCTGGTTCT
GGTGATGTTTGGATTGGTAAAACCTTGTATAGAATGCCAGAAATCTCCAACGATACCTAT
CATGAATTGGCTAAGACCGATTTCAAGAGATGTCAAGCTCAACATCAATTTGAATGGATC
TACATGCAAGAATGGTACGAATCTTGCAACATGGAAGAATTCGGTATCTCCAGAAAAGAA
TTATTGGTCGCTTACTTCTTGGCTACCGCTTCTATTTTTGAATTGGAAAGAGCCAACGAA
AGAATTGCTTGGGCTAAGTCTCAAATCATCTCTACTATTATCGCCTCCTTCTTCAACAAT
CAAAACACCTCTCCAGAAGATAAGTTGGCTTTCTTGACTGACTTTAAGAACGGTAACTCT
ACCAACATGGCTTTGGTTACTTTGACCCAATTCTTAGAAGGTTTCGACAGATACACTTCC
CACCAATTGAAAAATGCTTGGTCTGTTTGGTTGAGAAAGTTGCAACAAGGTGAAGGTAAT
GGTGGTGCTGATGCTGAATTATTAGTTAACACCTTGAACATTTGCGCCGGTCATATTGCT
TTCAGAGAAGAAATTTTGGCTCACAACGATTACAAGACCTTGTCTAACTTGACCTCTAAG
ATCTGCAGACAATTGAGTCAAATCCAAAACGAAAAAGAATTGGAAACCGAAGGTCAAAAG
ACCTCCATTAAGAACAAAGAATTAGAAGAAGATATGCAAAGATTAGTCAAGTTGGTCTTG
GAAAAGTCCAGAGTTGGTATCAACAGAGACATGAAGAAAACTTTCTTGGCCGTTGTTAAG
ACCTACTACTACAAAGCTTATCATTCCGCTCAAGCCATCGATAACCATATGTTTAAGGTT
TTGTTCGAACCAGTCGCCTGA
codon optimized DNA sequence encoding truncated CfTPS3:
SEQ ID NO: 20
ATGATCACCTCCAAATCTTCCGCTGCTGTTAAGTGTTCTTTGACTACTCCAACTGATTTG
ATGGGTAAGATCAAAGAAGTTTTCAACAGAGAAGTTGATACCTCTCCAGCTGCTATGACT
ACTCATTCTACTGATATTCCATCCAACTTGTGCATCATCGATACCTTGCAAAGATTGGGT
ATCGACCAATACTTCCAATCCGAAATTGATGCTGTCTTGCATGATACTTACAGATTGTGG
CAATTGAAGAAGAAGGACATCTTCTCTGATATTACCACTCATGCTATGGCCTTCAGATTA
TTGAGAGTTAAGGGTTACGAAGTTGCCTCTGATGAATTGGCTCCATATGCTGATCAAGAA
AGAATCAACTTGCAAACCATTGATGTTCCAACCGTCGTCGAATTATACAGAGCTGCACAA
GAAAGATTGACCGAAGAAGATTCTACCTTGGAAAAGTTGTACGTTTGGACTTCTGCTTTC
TTGAAGCAACAATTATTGACCGATGCCATCCCAGATAAGAAGTTGCATAAGCAAGTCGAA
TATTACTTGAAGAACTACCACGGTATCTTGGATAGAATGGGTGTTAGAAGAAACTTGGAC
TTGTACGATATCTCCCACTACAAATCTTTGAAGGCTGCTCATAGATTCTACAACTTGTCT
AACGAAGATATTTTGGCCTTCGCCAGACAAGATTTCAACATTTCTCAAGCCCAACACCAA
AAAGAATTGCAACAATTGCAAAGATGGTACGCCGATTGCAGATTGGATACTTTGAAATTC
GGTAGAGATGTCGTCAGAATCGGTAACTTTTTAACCTCTGCTATGATCGGTGATCCAGAA
TTGTCTGATTTGAGATTGGCTTTTGCTAAGCACATCGTTTTGGTTACCAGAATCGATGAT
TTCTTCGATCATGGTGGTCCAAAAGAAGAATCCTACGAAATTTTGGAATTGGTCAAAGAA
TGGAAAGAAAAGCCAGCTGGTGAATACGTTTCTGAAGAAGTCGAAATCTTATTCACCGCT
GTTTACAACACCGTTAACGAATTGGCTGAAATGGCCCATATTGAACAAGGTAGATCTGTT
AAGGATTTGTTGGTTAAGTTGTGGGTCGAAATATTGTCCGTTTTCAGAATCGAATTGGAT
ACCTGGACTAACGATACTGCTTTGACTTTGGAAGAATACTTGTCCCAATCCTGGGTTTCT
ATTGGTTGCAGAATCTGCATTTTGATCTCCATGCAATTCCAAGGTGTTAAGTTGAGTGAC
GAAATGTTGCAAAGTGAAGAATGTACCGATTTGTGCAGATACGTTTCCATGGTCGATAGA
TTATTGAACGATGTCCAAACCTTCGAAAAAGAAAGAAAAGAAAACACCGGTAACTCCGTT
TCTTTGTTGCAAGCTGCTCACAAAGACGAAAGAGTTATCAACGAAGAAGAAGCCTGCATC
AAGGTAAAAGAATTAGCCGAATACAATAGAAGAAAGTTGATGCAAATCGTCTACAAGACC
GGTACTATTTTCCCAAGAAAATGCAAGGACTTGTTCTTGAAGGCTTGTAGAATTGGTTGC
TACTTGTACTCTTCTGGTGATGAATTCACTTCCCCACAACAAATGATGGAAGATATGAAG
TCCTTGGTCTATGAACCATTGCCAATTTCTCCACCTGAAGCTAACAATGCATCTGGTGAA
AAAATGTCCTGCGTCAGTAACTGA
codon optimized DNA sequence encoding truncated ZmAN2:
SEQ ID NO: 21
ATGGCCCAACATACTTCTGAATCTGCTGCTGTTGCTAAAGGTTCTTCTTTGACTCCAATC
GTTAGAACCGATGCTGAATCTAGAAGAACTAGATGGCCAACAGATGATGATGACGCTGAA
CCATTGGTTGACGAAATTAGAGCTATGTTGACCTCTATGTCCGATGGTGATATTTCTGTT
TCTGCTTATGATACTGCTTGGGTTGGTTTGGTTCCAAGATTGGATGGTGGTGAAGGTCCA
CAATTTCCAGCTGCTGTTAGATGGATTAGAAACAATCAATTGCCAGATGGTTCTTGGGGT
GATGCTGCTTTGTTTTCAGCTTACGATAGATTGATTAACACCTTGGCTTGTGTTGTTACT
TTGACCAGATGGTCTTTGGAACCAGAAATGAGAGGTAGAGGTTTGTCTTTTTTGGGTAGA
AACATGTGGAAGTTGGCTACCGAAGATGAAGAATCTATGCCAATTGGTTTCGAATTGGCT
TTCCCATCCTTGATTGAATTGGCTAAATCTTTGGGTGTTCACGATTTCCCATATGATCAT
CAAGCTTTACAAGGTATCTACTCCTCCAGAGAAATCAAAATGAAGAGAATCCCAAAAGAA
GTCATGCATACTGTTCCAACCTCTATCTTGCATTCTTTGGAAGGTATGCCAGGTTTGGAT
TGGGCTAAGTTGTTGAAATTGCAATCCTCTGATGGTTCATTCTTGTTTTCACCAGCTGCT
ACTGCTTACGCTTTGATGAATACTGGTGATGATAGATGCTTCTCCTACATTGATAGAACC
GTCAAAAAGTTCAATGGTGGTGTTCCAAATGTTTACCCAGTTGACTTGTTTGAACATATC
TGGGCTGTTGACAGATTGGAAAGATTGGGTATTTCCAGATACTTCCAAAAAGAAATCGAA
CAATGCATGGACTACGTTAACAGACATTGGACTGAAGATGGTATTTGTTGGGCTAGAAAC
TCCGACGTAAAAGAAGTTGACGATACTGCTATGGCCTTCAGATTATTGAGATTGCATGGT
TACTCTGTTTCCCCAGATGTTTTCAAGAACTTCGAAAAGGATGGTGAATTCTTCGCTTTC
GTCGGTCAATCTAATCAAGCTGTTACTGGTATGTACAACTTGAACAGAGCCTCCCAAATT
TCATTTCCAGGTGAAGATGTTTTACACAGAGCTGGTGCTTTTTCTTACGAATTCTTGAGA
AGAAAAGAAGCCGAAGGTGCTTTGAGAGATAAGTGGATTATTTCCAAGGATTTGCCTGGT
GAAGTTGTCTACACTTTGGATTTTCCATGGTACGGTAATTTGCCAAGAGTTGAAGCTAGA
GACTACTTGGAACAATATGGTGGTGGTGATGACGTTTGGATAGGTAAAACATTATACAGA
ATGCCATTGGTCAACAACGACGTTTATTTGGAATTGGCCAGAATGGATTTCAACCATTGT
CAAGCCTTGCATCAATTGGAATGGCAAGGTTTGAAAAGATGGTACACCGAAAACAGATTG
ATGGATTTTGGTGTTGCTCAAGAAGATGCATTGAGAGCTTACTTTTTGGCTGCTGCTTCA
GTTTATGAACCATGTAGAGCTGCTGAAAGATTAGCTTGGGCAAGAGCTGCTATTTTGGCT
AATGCTGTTTCTACTCACTTGAGAAACTCTCCATCTTTCAGAGAAAGATTGGAACACTCT
TTGAGATGCAGACCTTCTGAAGAAACTGATGGTAGTTGGTTCAATTCCTCTTCTGGTTCT
GATGCTGTTTTGGTTAAGGCAGTTTTGAGATTGACTGATTCCTTGGCTAGAGAAGCTCAA
CCTATTCACGGTGGTGATCCAGAAGATATTATTCACAAGTTGTTAAGATCCGCTTGGGCT
GAATGGGTTAGAGAAAAAGCTGATGCTGCAGATTCTGTCTGTAATGGTTCTTCTGCTGTT
GAACAAGAAGGTTCCAGAATGGTTCATGATAAGCAAACCTGTTTGTTGTTGGCAAGAATG
ATTGAAATTTCCGCTGGTAGAGCCGCTGGTGAAGCTGCTTCCGAAGATGGTGACAGAAGA
ATTATACAATTGACCGGTTCCATCTGCGACTCATTGAAACAAAAAATGTTGGTCAGTCAA
GACCCAGAAAAGAACGAAGAAATGATGTCCCATGTTGACGACGAATTGAAGTTGAGAATC
AGAGAATTCGTCCAATACTTGTTGAGATTGGGTGAAAAAAAGACTGGTTCCTCTGAAACC
AGACAAACTTTCTTGTCTATCGTCAAGTCTTGTTACTACGCTGCTCATTGTCCACCACAT
GTTGTTGATAGACATATCTCCAGAGTTATCTTCGAACCAGTTTCTGCTGCTAAATTGGAA
CATCATCACCATCACCACTGA
codon optimized DNA sequence encoding truncated EpTPS1:
SEQ ID NO: 22
ATGGCTCAATCCGTTGCTGAATCCAACACCAGAATTCAACAATTGGATGGTACTAGAGAA
AAGATCAAGAAGATGTTCGACAAGGTCGAATTGTCTGTTTCTCCATATGATACTGCTTGG
GTTGCTATGGTTCCATCTCCAAATTCTTTGGAAGCTCCATACTTTCCAGAATGCTCTAAA
TGGATCGTCGACAATCAATTGAATGATGGTTCTTGGGGTTTCTACCATAGAGATCCATTA
TTGGTTAAGGACTCCATCTCTTCTACTTTGGCTTGTGTTTTGGCTTTGAAAAGATGGGGT
ATTGGTGAAAAGCAAGTCAACAAAGGTTTGGAATTCATCGAATTGAACTCCGCCTCTTTG
AACGATTTGAAACAATACAAGCCAGTCGGTTTCGATATTACCTTTCCAAGAATGTTGGAA
CACGCTAAGGATTTCGGTTTGAATTTGCCATTGGATCCTAAGTATGTTGAAGCCGTTATC
TTCTCCAGAGATTTGGATTTGAAATCCGGTTGTGATTCTACTACCGAAGGTAGAAAAGCT
TACTTGGCCTATATTTCCGAAGGTATCGGTAACTTGCAAGATTGGAATATGGTCATGAAG
TACCAAAGAAGAAACGGTTCCATTTTCGATTCTCCATCTGCTACAGCTGCTGCTTCTATT
CACTTGCATGATGCTTCATGTTTGAGATACTTGAGATGCGCCTTGAAGAAATTTGGTAAT
GCTGTTCCAACTATCTACCCATTCAACATCTACGTCAGATTGTCTATGGTTGATGCCATT
GAATCTTTGGGTATTGCCAGACACTTTCAAGAAGAAATCAAGACCGTTTTGGACGAAACT
TACAGATATTGGTTGCAAGGTAACGAAGAAATCTTCCAAGATTGCACTACTTGTGCTATG
GCCTTCAGAATTTTGAGAGCTAATGGTTACAACGTTTCCTCCGAAAAGTTGAATCAATTC
ACCGAAGATCACTTCTCCAATTCATTGGGTGGTTATTTGGAAGATATGAGACCAGTCTTG
GAATTATACAAGGCCTCCCAATTGATTTTCCCAGACGAATTATTCTTAGAAAAGCAATTC
TCCTGGACCTCCCAATGTTTGAAGCAAAAAATCTCTTCCGGTTTGAGACATACCGACGGT
ATTAACAAACACATTACCGAAGAAGTTAACGACGTTTTGAAGTTCGCTTCTTACGCTGAT
TTGGAAAGATTGACCAATTGGAGAAGAATCGCTGTTTACAGAGCTAACGAAACAAAAATG
TTGAAAACCTCCTACAGATGCTCCAACATTGCTAACGAACACTTTTTGGAATTGGCCGTC
GAAGATTTCAACGTTTGTCAATCAATGCACAGAGAAGAATTGAAGCACTTGGGTAGATGG
GTTGTTGAAAAGAGATTGGACAAGTTGAAATTCGCCAGACAAAAGTTGGGTTACTGCTAC
TTTTCTTCAGCTGCTTCTTTGTTTGCTCCAGAAATGTCTGATGCTAGAATTTCTTGGGCT
AAGAATGCCGTTTTGACTACCGTTGTTGATGACTTTTTTGATGTCGGTGGTTCCGAAGAA
GAATTGATTAACTTGGTCCAATTGATCGAAAGATGGGACGTTGATGGTTCCTCTCATTTC
TGTTCTGAACATGTCGAAATCGTTTTCTCTGCCTTGCATTCTACCATTTGCGAAATAGGT
GAAAAGGCTTTTGCTTATCAAGGTAGAAGAATGACCTCCCACGTTATTAAGATTTGGTTG
GACTTGTTGAAGTCCATGTTGACTGAAACTTTGTGGTCTAAGTCTAAGGCTACTCCAACC
TTGAACGAATATATGACTAACGGTAACACCTCTTTTGCTTTGGGTCCAATAGTTTTGCCA
GCTTTGTTTTTTGTTGGTCCAAAGTTGACCGACGAAGATTTGAAGTCTCATGAATTGCAC
GATTTGTTCAAGACCATGTCTACCTGTGGTAGATTATTGAACGATTGGAGATCCTACGAA
AGAGAATCTGAAGAAGGTAAATTGAACGCCGTTTCCTTGCATATGATCTACGGTAATGGT
TCTGTTGCTGCTACTGAAGAAGAAGCTACTCAAAAGATTAAGGGTTTGATCGAATCCGAA
AGAAGAGAATTGATGAGATTGGTATTGCAAGAAAAGGACTCTAAGATTCCTAGACCATGC
AAGGATTTGTTCTGGAAGATGTTGAAGGTCTTGCACATGTTCTACTTGAAGGATGATGGT
TTCACCTCCAATCAAATGATGAAGACTGCTAACTCCTTGATCAATCAACCTATCTCATTG
CACGAAAGAGTTGAACATCATCATCACCATCACTAA
codon optimized DNA sequence encoding truncated TwTPS21:
SEQ ID NO: 23
ATGGGTATCGCTAAATCCAAGCCAGCTAGAACTACTCCAGAATACTCTGATGTTTTACAA
ACTGGTTTGCCATTGATCGTCGAAGATGATATCCAAGAACAAGAAGAACCATTGGAAGTT
TCTTTGGAAAATCAAATCAGACAAGGTGTCGACATCGTCAAATCTATGTTGGGTTCTATG
GAAGATGGTGAAACCTCTATTTCTGCTTATGATACTGCTTGGGTTGCCTTGGTTGAAAAC
ATTCATCATCCAGGTAGTCCACAATTCCCATCTTCATTACAATGGATCGCCAACAATCAA
TTGCCAGATGGTTCTTGGGGTGATCCAGATGTTTTTTTGGCTCATGATAGATTGATTAAC
ACCTTGGCTTGCGTTATTGCTTTGAAGAAGTGGAATATCCATCCACACAAATGCAAGAGA
GGTTTGTCTTTCGTCAAAGAAAACATTTCTAAGTTGGAAAAAGAAAACGAAGAACACATG
TTGATCGGTTTCGAAATTGCCTTTCCATCCTTGTTGGAAATGGCTAAGAAATTGGGTATC
GAAATCCCAGATGATTCTCCAGCTTTACAAGATATCTACACCAAGAGAGATTTGAAGTTG
ACCAGAATCCCAAAGGATAAGATGCATAACGTTCCAACTACCTTGTTGCATTCATTGGAA
GGTTTGCCAGATTTGGATTGGGAAAAGTTGGTTAAGTTGCAATTCCAAAACGGTTCCTTT
TTGTTCTCTCCATCTTCTACTGCTTTTGCCTTTATGCATACCAAGGATGGTAACTGCTTG
TCCTACTTGAATGATTTGGTTCACAAGTTCAATGGTGGTGTTCCAACTGCTTATCCAGTT
GATTTGTTTGAACACATCTGGTCCGTTGACAGATTGCAAAGATTGGGTATTTCCAGATTC
TTCCACCCAGAAATCAAAGAATGTTTGGGTTACGTTCATAGATACTGGACTAAGGACGGT
ATTTGTTGGGCTAGAAATTCCAGAGTTCAAGATATTGATGATACCGCCATGGGTTTCAGA
TTATTGAGATTGCATGGTTACGAAGTTTCCCCAGATGTCTTTAAGCAATTCAGAAAGGGT
GATGAATTCGTCTGTTTCATGGGTCAATCCAATCAAGCTATTACCGGTATCTACAACTTG
TACAGAGCTTCCCAAATGATGTTCCCAGAAGAAACCATTTTGGAAGAAGCCAAGAAGTTC
TCCGTTAACTTCTTGAGAGAAAAGAGAGCTGCCTCTGAATTATTGGATAAGTGGATTATC
ACCAAGGACTTGCCAAATGAAGTTGGTTTTGCTTTGGATGTTCCATGGTATGCTTGTTTG
CCAAGAGTTGAAACCAGATTGTACATCGAACAATACGGTGGTCAAGATGATGTTTGGATA
GGTAAGACCTTGTATAGAATGCCATACGTCAACAACAACGTCTACTTGGAATTGGCCAAA
TTGGATTACAACAACTGCCAATCCTTGCACAGAATTGAATGGGACAATATCCAAAAGTGG
TACGAAGGTTACAATTTGGGTGGTTTTGGTGTCAACAAGAGATCCTTATTGAGAACCTAC
TTTTTGGCCACCTCCAACATTTTTGAACCAGAAAGATCTGTCGAAAGATTGACTTGGGCT
AAGACTGCTATTTTGGTTCAAGCCATTGCTTCCTACTTCGAAAACTCTAGAGAAGAAAGA
ATCGAATTCGCCAACGAATTTCAAAAGTTCCCAAACACTAGAGGTTACATCAACGGTAGA
AGATTGGATGTTAAGCAAGCTACCAAGGGTTTGATCGAAATGGTTTTCGCTACCTTGAAT
CAATTCTCCTTGGATGCCTTAGTTGTTCACGGTGAAGATATTACTCATCACTTGTACCAA
TCCTGGGAAAAATGGGTTTTGACTTGGCAAGAAGGTGGTGATAGAAGAGAAGGTGAAGCC
GAATTATTAGTCCAAACCATTAACTTGATGGCCGGTCATACTCATAGTCAAGAAGAAGAA
TTATACGAAAGATTATTCAAGTTGACTAACACCGTCTGCCATCAATTGGGTCATTATCAT
CATTTGAACAAGGATAAGCAACCACAACAAGTCGAAGATAATGGTGGTTACAACAATTCC
AACCCAGAATCCATCTCCAAGTTGCAAATTGAATCCGACATGAGAGAATTGGTCCAATTG
GTTTTGAACTCCTCTGATGGTATGGACTCTAACATCAAGCAAACTTTCTTGGCTGTTACC
AAGTCTTTCTACTACACTGCTTTTACTCATCCTGGTACTGTCAACTACCATATTGCTAAG
GTTTTGTTCGAAAGAGTCGTCTTAGAACATCATCATCACCATCACTGA
codon optimized DNA sequence encoding truncated SsSCS:
SEQ ID NO: 24
ATGTCCTTGGCTTTCAACGTTGGTGTTACTCCATTTTCTGGTCAAAGAGTCGGTTCCAGA
AAAGAAAAGTTTCCAGTTCAAGGTTTCCCAGTTACTACTCCAAATAGATCCAGATTGATC
GTCAACTGTTCCTTGACTACCATTGATTTCATGGCCAAGATGAAGGAAAACTTCAAGAGA
GAAGATGACAAGTTCCCAACTACTACTACCTTGAGATCTGAAGATATCCCATCCAACTTG
TGCATTATCGATACCTTGCAAAGATTGGGTGTTGACCAATTCTTCCAATACGAAATCAAC
ACCATCTTGGACAACACTTTCAGATTGTGGCAAGAAAAGCACAAGGTTATCTACGGTAAT
GTTACTACACATGCTATGGCCTTCAGATTATTGAGAGTTAAGGGTTACGAAGTTTCCTCC
GAAGAATTAGCTCCATACGGTAATCAAGAAGCCGTTTCTCAACAAACTAACGACTTGCCA
ATGATCATCGAATTATACAGAGCTGCCAACGAAAGAATCTACGAAGAAGAAAGATCCTTG
GAAAAGATTTTGGCTTGGACCACCATTTTCTTGAACAAGCAAGTTCAAGACAACTCCATC
CCAGATAAGAAGTTGCATAAGTTGGTCGAATTCTACTTGAGAAACTACAAGGGTATCACC
ATTAGATTAGGTGCCAGAAGAAACTTGGAATTATACGACATGACTTACTACCAAGCCTTG
AAGTCTACCAACAGATTCTCTAACTTGTGTAACGAAGATTTCTTGGTTTTCGCCAAGCAA
GATTTCGATATTCACGAAGCCCAAAATCAAAAGGGTTTACAACAATTACAAAGATGGTAC
GCCGATTGCAGATTGGATACTTTGAATTTCGGTAGAGATGTCGTCATTATCGCTAACTAT
TTGGCCTCCTTGATTATTGGTGATCATGCCTTTGATTACGTCAGATTGGCTTTTGCTAAG
ACCTCTGTTTTGGTTACCATCATGGATGATTTCTTCGATTGCCATGGTTCTTCTCAAGAA
TGCGACAAGATAATCGAATTGGTAAAAGAATGGAAAGAAAACCCAGATGCCGAATACGGT
TCTGAAGAATTGGAAATTTTGTTCATGGCCTTGTACAACACCGTTAACGAATTGGCTGAA
AGAGCTAGAGTTGAACAAGGTAGATCTGTCAAAGAATTTTTGGTCAAGTTGTGGGTTGAA
ATCTTGTCCGCTTTCAAGATTGAATTGGATACCTGGTCTAACGGTACTCAACAATCTTTC
GACGAATATATCTCCTCCTCTTGGTTGTCTAATGGTTCTAGATTGACTGGTTTGTTGACC
ATGCAATTTGTTGGTGTCAAATTGTCCGACGAAATGTTGATGTCAGAAGAATGTACTGAT
TTGGCTAGACACGTATGTATGGTCGGTAGATTATTGAACGATGTCTGCTCATCTGAAAGA
GAAAGAGAAGAAAACATTGCCGGTAAGTCCTACTCTATTTTGTTGGCTACTGAAAAGGAC
GGTAGAAAGGTTTCTGAAGATGAAGCTATTGCTGAAATCAACGAAATGGTCGAATACCAT
TGGAGAAAGGTCTTGCAAATCGTCTACAAGAAAGAATCCATCTTGCCTAGAAGATGCAAG
GACGTTTTTTTGGAAATGGCTAAGGGTACTTTTTACGCCTACGGTATTAACGATGAATTG
ACCTCTCCACAACAATCCAAAGAAGATATGAAGTCCTTCGTTTTTTAA
codon optimized DNA sequence encoding truncated TwTPS14:
SEQ ID NO: 25
ATGTTTATGTCCTCCTCCTCATCCTCTCATGCTAGAAGACCACAATTGTCATCTTTCTCT
TACTTGCATCCACCATTGCCATTTCCAGGTTTGTCATTTTTCAACACCAGAGACAAGAGA
GTCAACTTCGATTCTACCAGAATTATCTGCATTGCCAAATCTAAGCCAGCTAGAACTACT
CCAGAATACTCCGATGTTTTACAAACTGGTTTGCCATTGATCGTCGAAGATGATATCCAA
GAACAAGAAGAACCATTGGAAGTTTCTTTGGAAAATCAAATCAGACAAGGTGTCGACATC
GTCAAATCTATGTTGGGTTCTATGGAAGATGGTGAAACCTCTATTTCTGCTTATGATACT
GCTTGGGTTGCCTTGGTTGAAAACATTCATCATCCAGGTAGTCCACAATTCCCATCTTCA
TTACAATGGATCGCCAACAATCAATTGCCAGATGGTTCTTGGGGTGATCCAGATGTTTTT
TTGGCTCATGATAGATTGATTAACACCTTGGCTTGCGTTATTGCTTTGAAGAAGTGGAAT
ATCCATCCACACAAATGCAAGAGAGGTTTGTCTTTCGTCAAAGAAAACATTTCTAAGTTG
GAAAAAGAAAACGAAGAACACATGTTGATCGGTTTCGAAATTGCCTTTCCATCCTTGTTA
GAAATGGCTAAGAAGTTGGGTATCGAAATCCCAGATGATTCTCCAGCTTTACAAGATATC
TACACCAAGAGAGATTTGAAGTTGACCAGAATCCCAAAGGATATCATGCATAACGTTCCA
ACTACCTTGTTGTACTCTTTGGAAGGTTTGCCTTCTTTGGATTGGGAAAAGTTGGTTAAG
TTGCAATGTACTGACGGTTCCTTTTTGTTCTCTCCATCTTCTACTGCTTGTGCTTTGATG
CATACAAAAGATGGTAACTGCTTCTCCTACATCAACAACTTGGTCCATAAGTTTAATGGT
GGTGTTCCAACTGTTTACCCAGTTGATTTGTTTGAACATATCTGGTGCGTTGACAGATTG
CAAAGATTGGGTATTTCCAGATTCTTCCACCCAGAAATCAAAGAATGTTTGGGTTACGTT
CATAGATACTGGACCAAGGATGGTATTTGTTGGGCTAGAAATTCCAGAGTTCAAGATATT
GATGATACCGCCATGGGTTTCAGATTATTGAGATTGCATGGTTACGAAGTTTCCCCAGAT
GTCTTTAAGCAATTCAGAAAGGGTGATGAATTCGTCTGTTTCATGGGTCAATCCAATCAA
GCTATTACCGGTATCTACAACTTGTACAGAGCTTCCCAAATGATGTTCCCAGAAGAAACC
ATTTTGGAAGAAGCCAAGAAGTTCTCCGTTAACTTCTTGAGAGAAAAGAGAGCTGCCTCT
GAATTATTGGATAAGTGGATTATCACCAAGGACTTGCCAAATGAAGTTGGTTTTGCTTTG
GATGTTCCATGGTATGCTTGTTTGCCAAGAGTTGAAACCAGATTGTACATCGAACAATAC
GGTGGTCAAGATGATGTTTGGATAGGTAAGACCTTGTATAGAATGCCATACGTCAACAAC
AACGTCTACTTGGAATTGGCCAAATTGGATTACAACAACTGCCAATCCTTGCACAGAATT
GAATGGGACAATATCCAAAAGTGGTACGAAGGTTACAATTTGGGTGGTTTTGGTGTCAAC
AAGAGATCCTTATTGAGAACCTACTTTTTGGCCACCTCCAACATTTTTGAACCAGAAAGA
TCTGTCGAAAGATTGACTTGGGCTAAGACTGCTATTTTGGTTCAAGCCATTGCTTCCTAC
TTCGAAAACTCTAGAGAAGAAAGAATCGAATTCGCCAACGAATTCCAAAAGTTCCCAAAC
ACTAGAGGTTACATCAACGGTAGAAGATTGGATGTTAAGCAAGCTACCAAGGGTTTGATC
GAAATGGTTTTCGCTACCTTGAATCAATTCTCCTTGGATGCATTGGTTGTTCACGGTGAA
GATATTACTCATCACTTGTACCAATCCTGGGAAAAATGGGTTTTGACTTGGCAAGAAGGT
GGTGATAGAAGAGAAGGTGAAGCCGAATTATTAGTCCAAACCATTAACTTGATGGCCGGT
CATACTCATAGTCAAGAAGAAGAATTATACGAAAGATTATTCAAGTTGACTAACACCGTC
TGCCATCAATTGGGTCATTATCATCATTTGAACAAGGACAAGCAACCACAACAAGTCGAA
GATAACGGTGGTTACAACAATTCTAACCCAGAATCCATCTCCAAGTTGCAAATCGAATCT
GACATGAGAGAATTGGTCCAATTGGTCTTGAATTCCTCTGATGGTATGGACTCTAACATC
AAGCAAACTTTCTTGGCTGTTACCAAGTCTTTCTACTACACTGCTTTTACTCATCCTGGT
ACTGTCAACTACCATATTGCTAAGGTTTTGTTCGAAAGAGTTGTTTAA
MvTPS1
SEQ ID NO: 28
MASTPTLNLSITTPFVRTKIPAKISLPACSWLDRSSSRHVELNHKFCRKLELKVAMCRAS
LDVQQVRDEVYSNAQPHELVDKKIEERVKYVKNLLSTMDDGRINWSAYDTAWISLIKDFE
GRDCPQFPSTLERIAENQLPDGSWGDKDFDCSYDRIINTLACVVALTTWNVHPEINQKGI
RYLKENMRKLEETPTVLMTCAFEVVFPALLKKARNLGIHDLPYDMPIVKEICKIGDEKLA
RIPKKMMEKETTSLMYAAEGVENLDWERLLKLRTPENGSFLSSPAATVVAFMHTKDEDCL
RYIKYLLNKFNGGAPNVYPVDLWSRLWATDRLQRLGISRYFESEIKDLLSYVHSYWTDIG
VYCTRDSKYADIDDTSMGFRLLRVQGYNMDANVFKYFQKDDKFVCLGGQMNGSATATYNL
YRAAQYQFPGEQILEDARKFSQQFLQESIDTNNLLDKWVISPHIPEEMRFGMEMTWYSCL
PRIEASYYLQHYGATEDVWLGKTFFRMEEISNENYRELAILDFSKCQAQHQTEWIHMQEW
YESNNVKEFGISRKDLLFAYFLAAASIFETERAKERILWARSKIICKMVKSFLEKETGSL
EHKIAFLTGSGDKGNGPVNNAMATLHQLLGEFDGYISIQLENAWAAWLTKLEQGEANDGE
LLATTINICGGRVNQDTLSHNEYKALSDLINKICHNLAQIQNDKGDEIKDSKRSERDKEV
EQDMQALAKLVFEESDLERSIKQTFLAVVRTYYYGAYIAAEKIDVHMFKVLFKPVG*

| | |
|---|---|
| SEQ ID NO: 1 | Amino acid sequence of syn-CPP from Oryza sativa |
| SEQ ID NO: 2 | Amino acid sequence of TPS7 from Euphobia peplus |
| SEQ ID NO: 3 | Amino acid sequence of AN2 from Zea Maiz |
| SEQ ID NO: 4 | Amino acid sequence of TPS7 from |
| | Tripterygium Wilfordii |
| SEQ ID NO: 5 | Amino acid sequence of TPS1 from Coleus forskohlii |
| SEQ ID NO: 6 | Amino acid sequence of LPPS from Salvia scarea |
| SEQ ID NO: 7 | Amino acid sequence of TPS21 from |
| | Tripterygium Wilfordii |
| SEQ ID NO: 8 | Amino acid sequence of TPS14/28 from |
| | Tripterygium Wilfordii |
| SEQ ID NO: 9 | Amino acid sequence of TPS8 of Euphobia peplus |
| SEQ ID NO: 10 | Amino acid sequence of TPS23 of Euphobia peplus |
| SEQ ID NO: 11 | Amino acid sequence of SCS of Salvia scarea |
| SEQ ID NO: 12 | Amino acid sequence of TPS3 of Coleus forskohlii |
| SEQ ID NO: 13 | Amino acid sequence of TPS4 of Coleus forskohlii |
| SEQ ID NO: 14 | Amino acid sequence of TPS2 of |
| | Tripterygium Wilfordii |
| SEQ ID NO: 15 | Amino acid sequence of TPS1 of Euphobia peplus |
| SEQ ID NO: 16 | Amino acid sequence of TPS14 of Coleus forskohlii |
| SEQ ID NO: 17 | Amino acid sequence of TPS2 of Coleus forskohlii |
| SEQ ID NO: 18 | Amino acid sequence of TPS5 from |
| | Marrubium vulgare |
| SEQ ID NO: 19 | DNA sequence encoding truncated CfTPS1 codon |
| | optimised for expression in Saccharomyzes cerevisae |
| SEQ ID NO: 20 | DNA sequence encoding truncated CfTPS3 codon |
| | optimised for expression in Saccharomyzes cerevisae |
| SEQ ID NO: 21 | DNA sequence encoding truncated ZmAN2 codon |
| | optimised for expression in Saccharomyzes cerevisae |
| SEQ ID NO: 22 | DNA sequence encoding truncated EpTPS1 codon |
| | optimised for expression in Saccharomyzes cerevisae |
| SEQ ID NO: 23 | DNA sequence encoding truncated TwTPS21 codon |
| | optimised for expression in Saccharomyzes cerevisae |
| SEQ ID NO: 24 | DNA sequence encoding truncated SsSCS codon |
| | optimised for expression in Saccharomyzes cerevisae |
| SEQ ID NO: 25 | DNA sequence encoding truncated TwTPS14 codon |
| | optimised for expression in Saccharomyzes cerevisae |
| SEQ ID NO: 26 | Amino acid sequence of DXS of Coleus forskohlii |
| SEQ ID NO: 27 | Amino acid sequence of GGPPS of Coleus forskohlii |
| SEQ ID NO: 28 | Amino acid sequence of TPS1 of Marrubium vulgare |

### EXAMPLES

The invention is further illustrated by the following examples, which however, should not be construed as limiting for the invention.

### Example 1

Full length cDNAs encoding 9 class II diTPS and 9 class I diTPS were cloned from a library of full length cDNAs. Sequences of cDNAs were determined by deep sequencing according to standard methods and putative diTPS were selected based on phylogeny essentially as described in Zerbe, Hamberger et al. 2013.

The 9 class II diTPSs catalyse formation of 6 structurally and stereochemically distinct diterpene pyrophosphate intermediates (see FIG. 3). The 9 class I diTPSs convert the diterpene pyrophosphate intermediates to the diterpenes. When these enzymes are expressed heterologously in E. coli, yeast or the Nicotiana benthamiana/Agrobacterium systems in combinations of specific class II and class I enzymes, it was found that even combinations of diTPS class II and class I enzymes not found in nature, would lead to production of at least 47 individual diterpenes including previously described and novel diterpenes. The individual diterpenes were detected with GC-MS and LC-MS in extracts derived from the cells overexpressing the diTPS as described below.

Transient Expression in N. Benthamiana

Putative diTPS enzymes were expressed using the previously described pCAMBIA130035Su vector. pCAMBIA130035Su containing nucleic acids encoding putative diTPS and T-DNA expression plasmid containing the anti-post transcriptional gene silencing protein p19 (35S:p19)(Voinnet, Rivas et al. 2003), were transformed into the AGL-1-GV3850 Agrobacterium strain by electroporation using a 2 mm electroporation cuvette in a Gene Pulser (Bio-Rad; Capacity 25 μF; 2.5 kV; 400Ω). The transformed agrobacteria were subsequently transferred to 1 mL YEP (yeast extract peptone) media and grown for 2-3 hours at 30° C. in YEP media. 200 μL were transferred to YEP-agar solid media containing 35 μg/mL rifampicillin, 50 μg/mL carbencillin and 50 μg/mL kanamycin and grown for 2 days. Multiple colonies were transferred from the plate to 20 mL YEP media in falcon tube containing 17.5 μg/mL rifampicillin, 25 μg/mL carbencillin and 25 μg/mL kanamycin and grown at 30° C. over night (ON) at 225 rpm. Agrobacteria were spun down and by centriguation at 3500×g for 10 min and resuspended in 5 mL H₂O. OD₆₀₀ were measured and H₂O was added to reach an OD₆₀₀=1.3 mL of agrobacteria culture containing the plasmid with nucleic acids encoding putative diTPS class II, diTPS class I and p19 gene respectively was mixed. Controls only containing either diTPS class II, diTPS class I or p19 was mixed similarly. Each mix of agrobacteria cultures were infiltrated into independent 4-6 weeks old N. benthamiana plants. In total 121 independent N. benthamiana lines were made. Plants were grown for 7 days in greenhouse before metabolite extraction.

Extraction and GC-MS Analysis

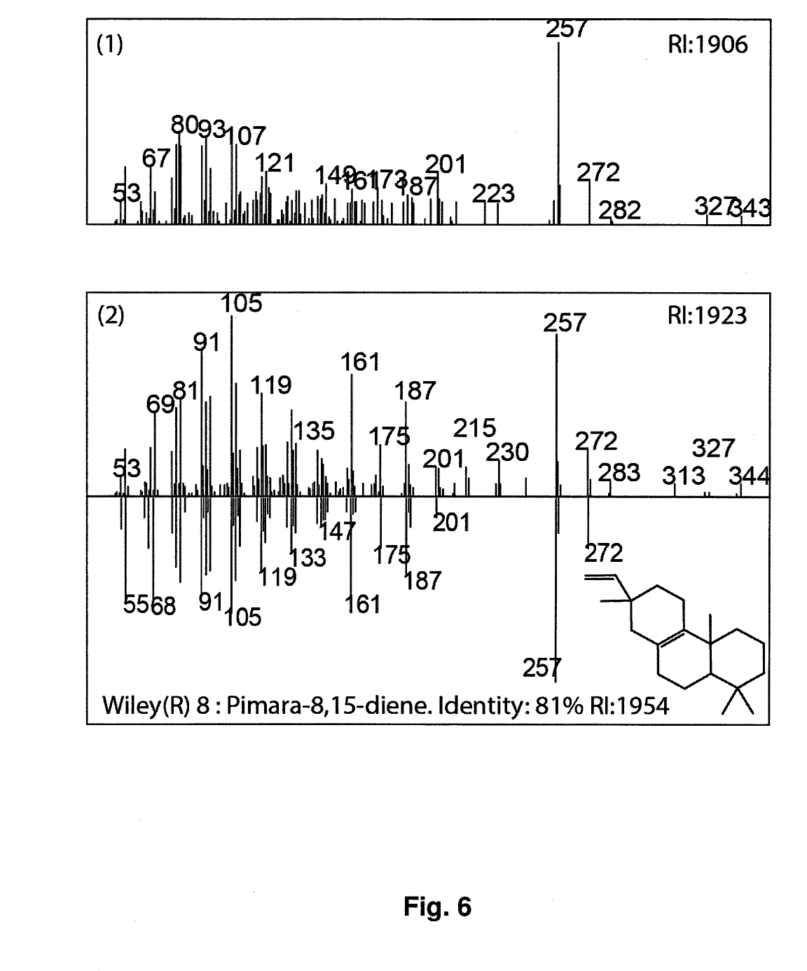

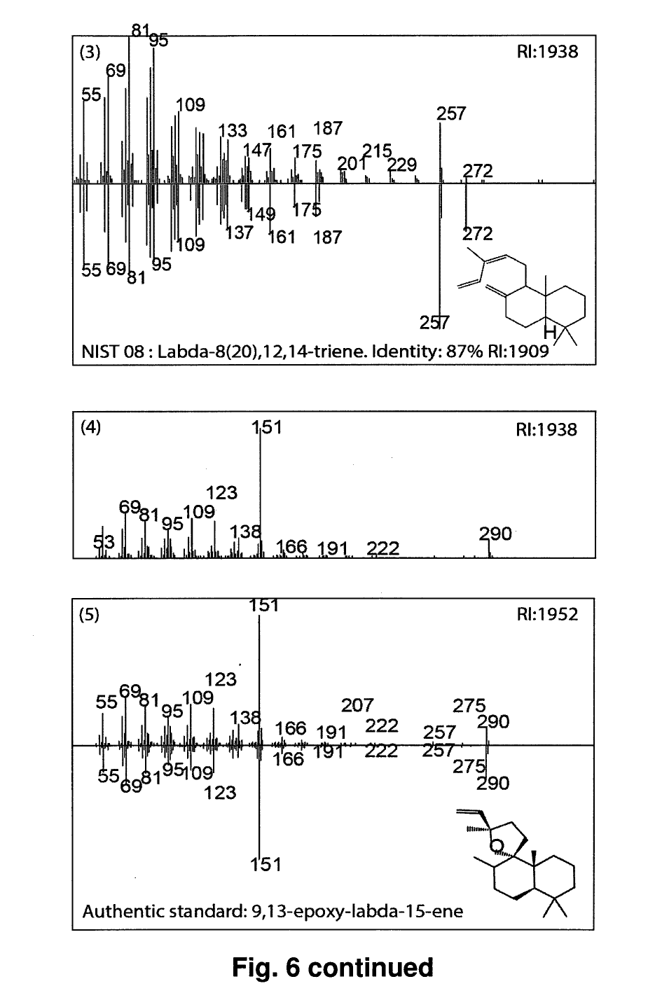

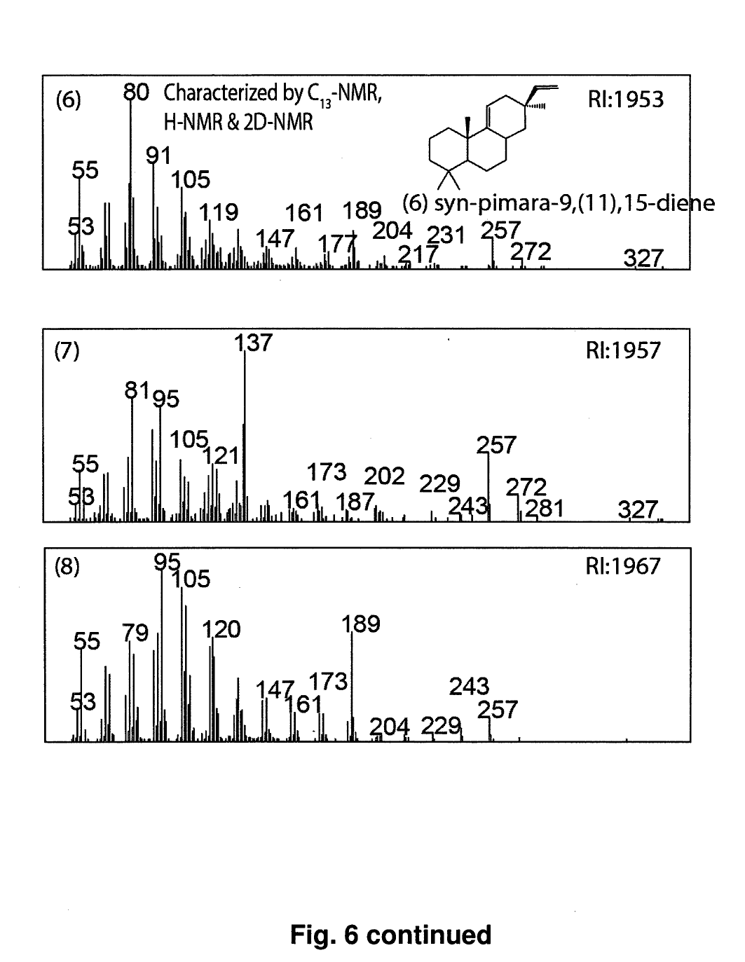

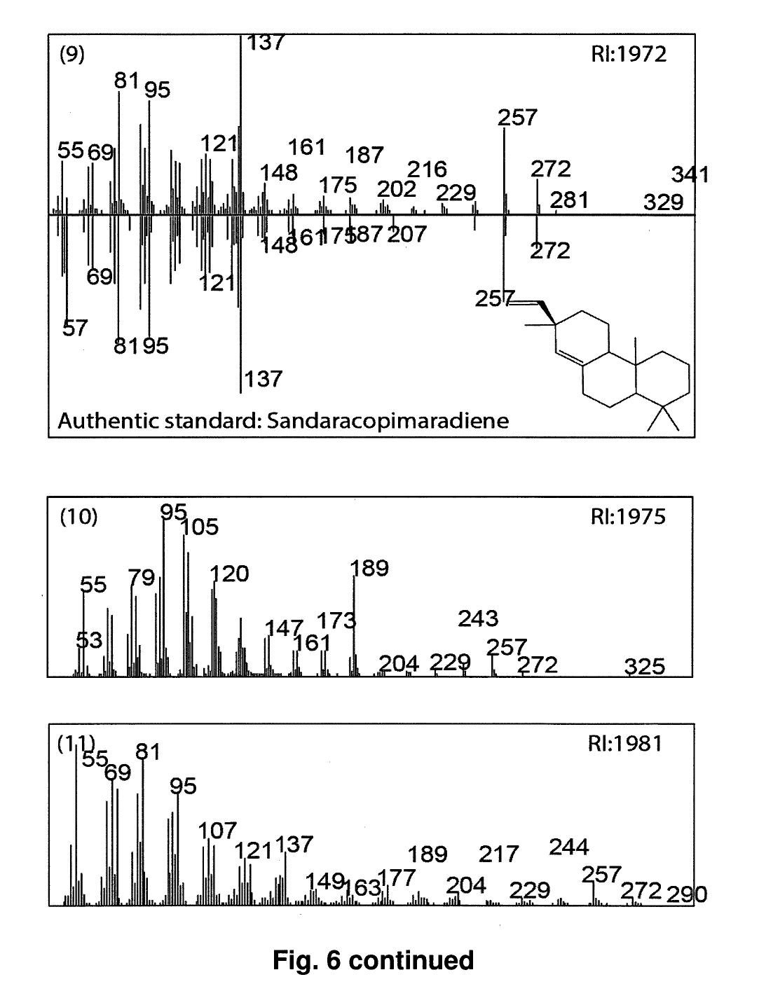

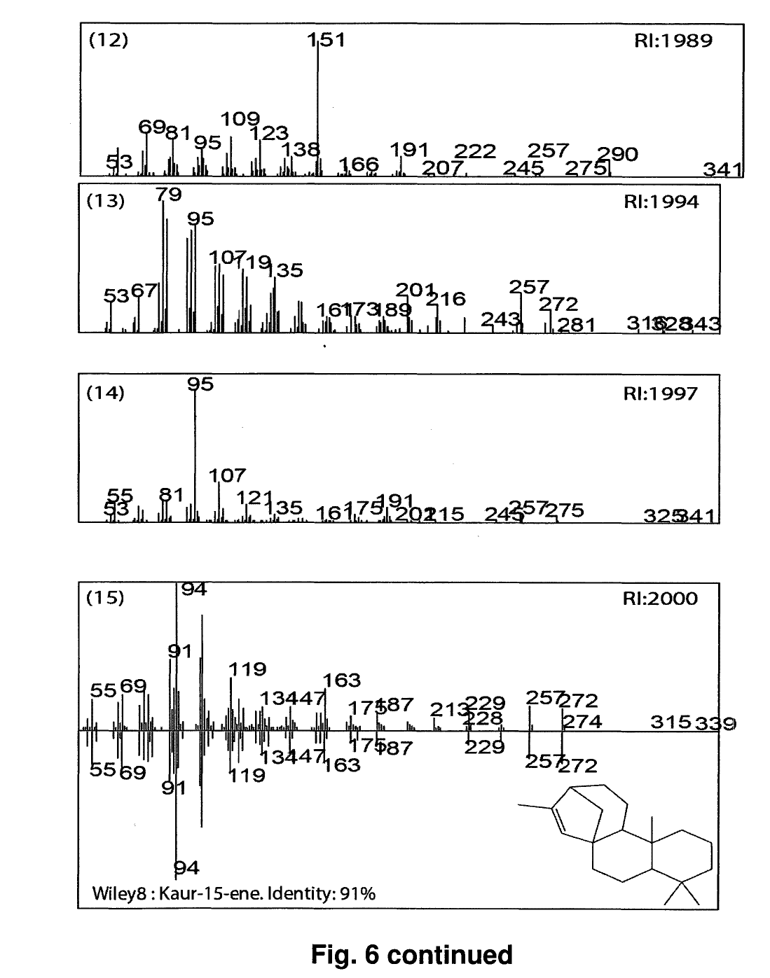

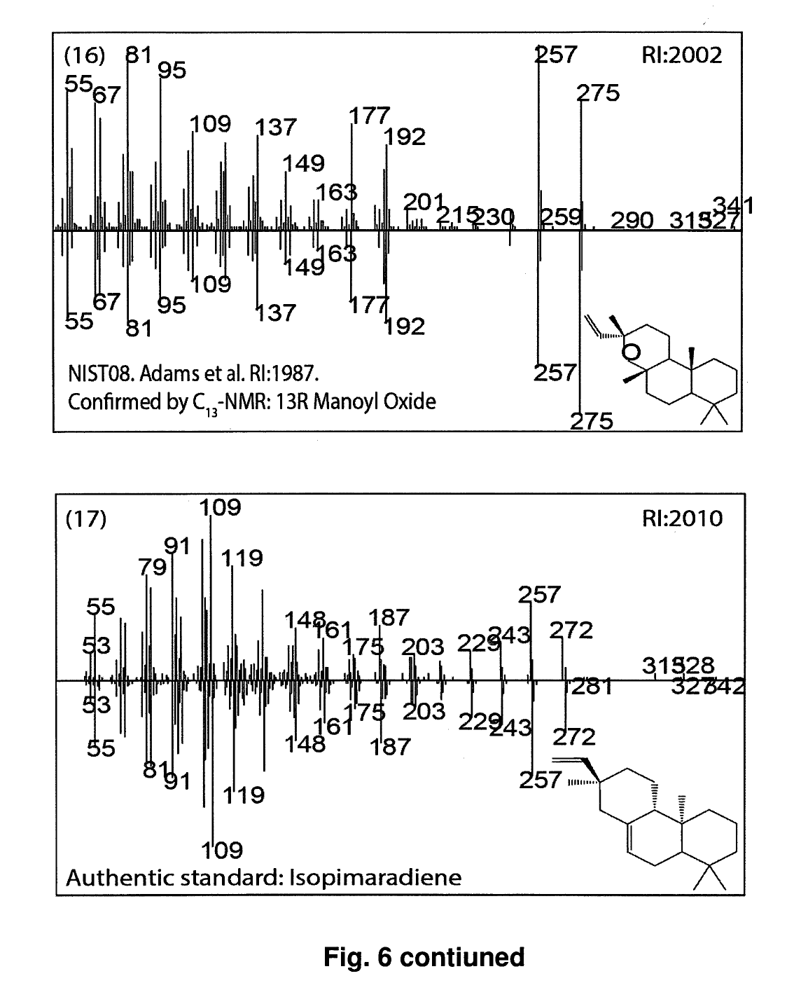

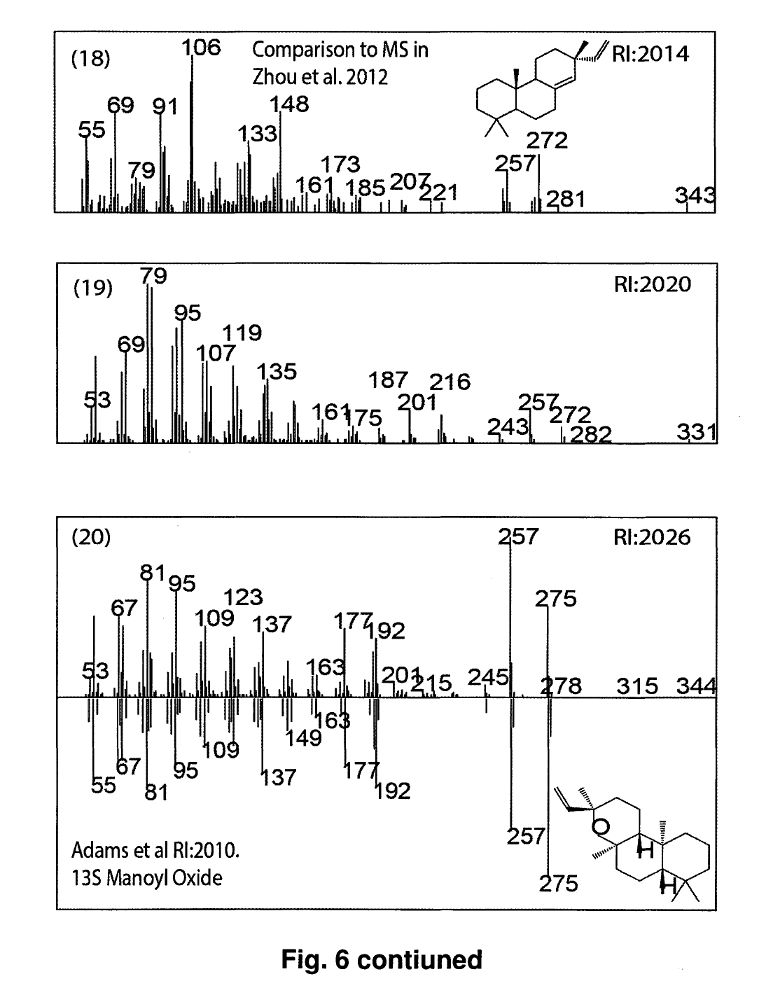

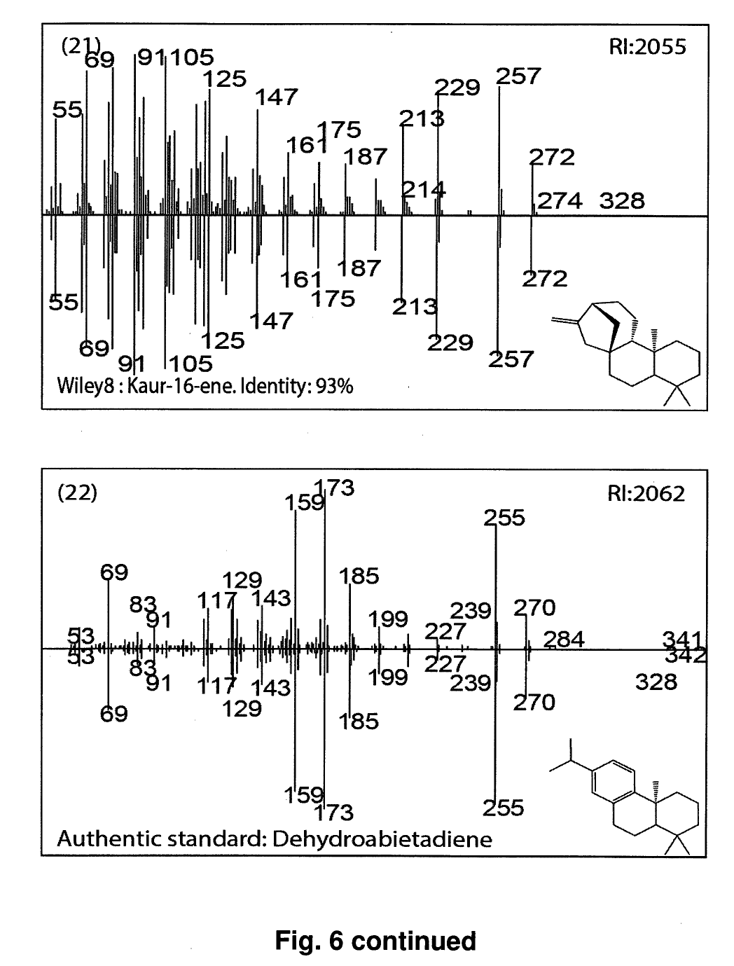

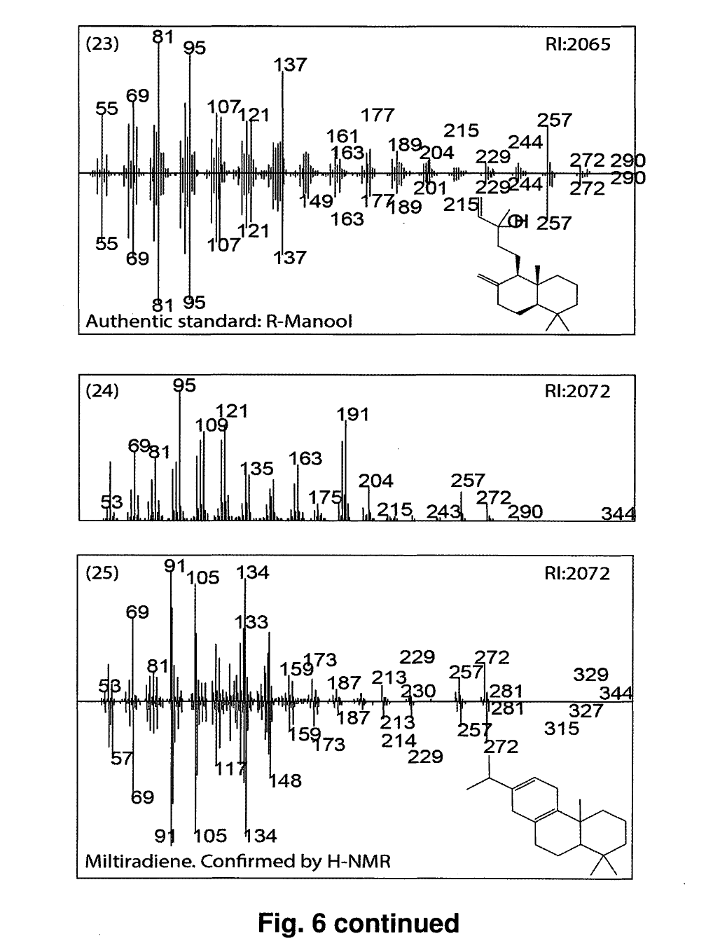

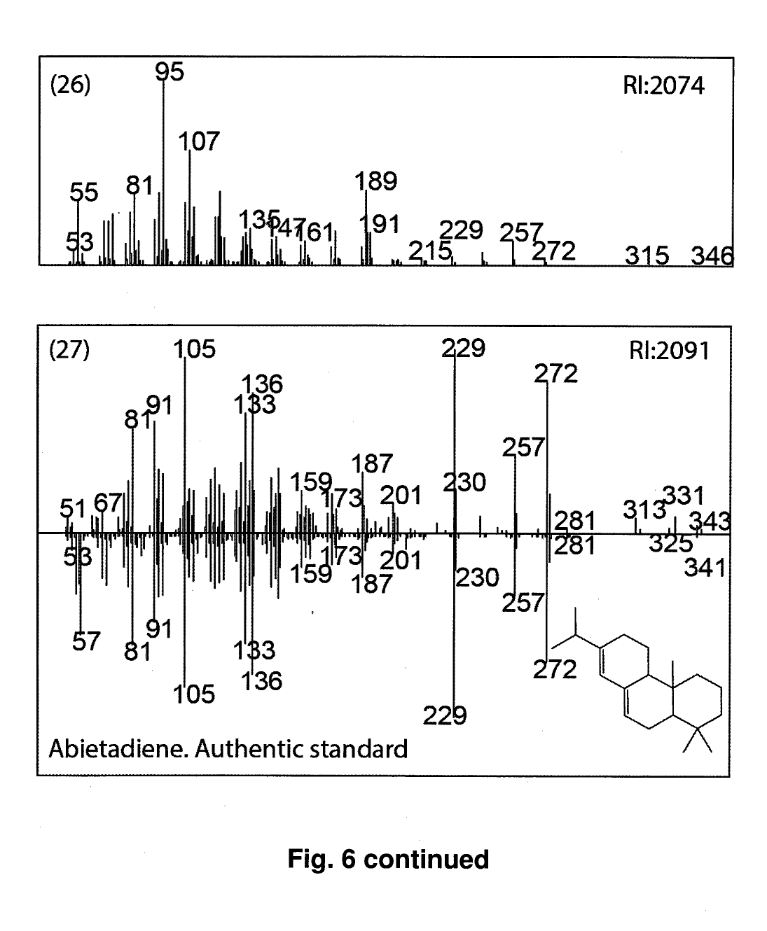

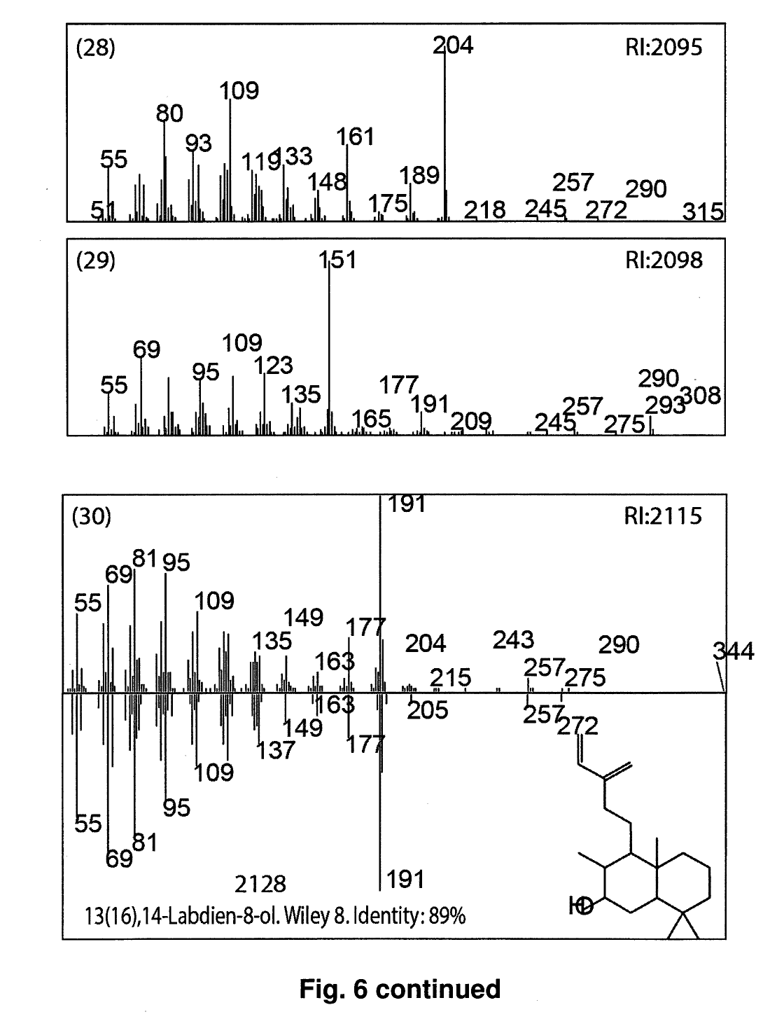

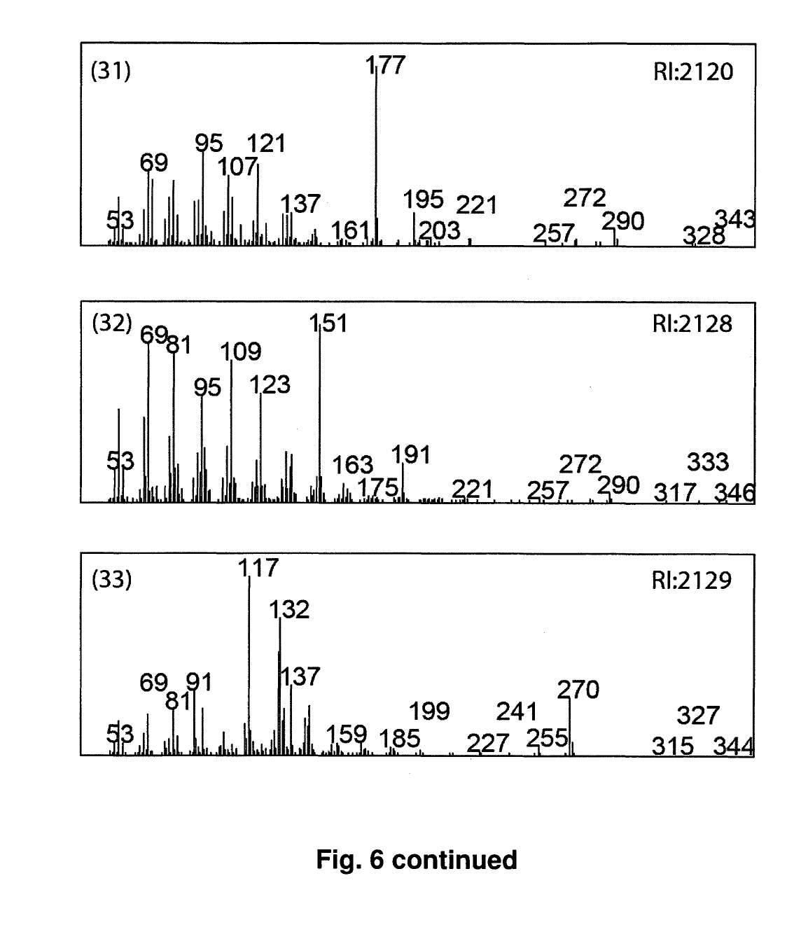

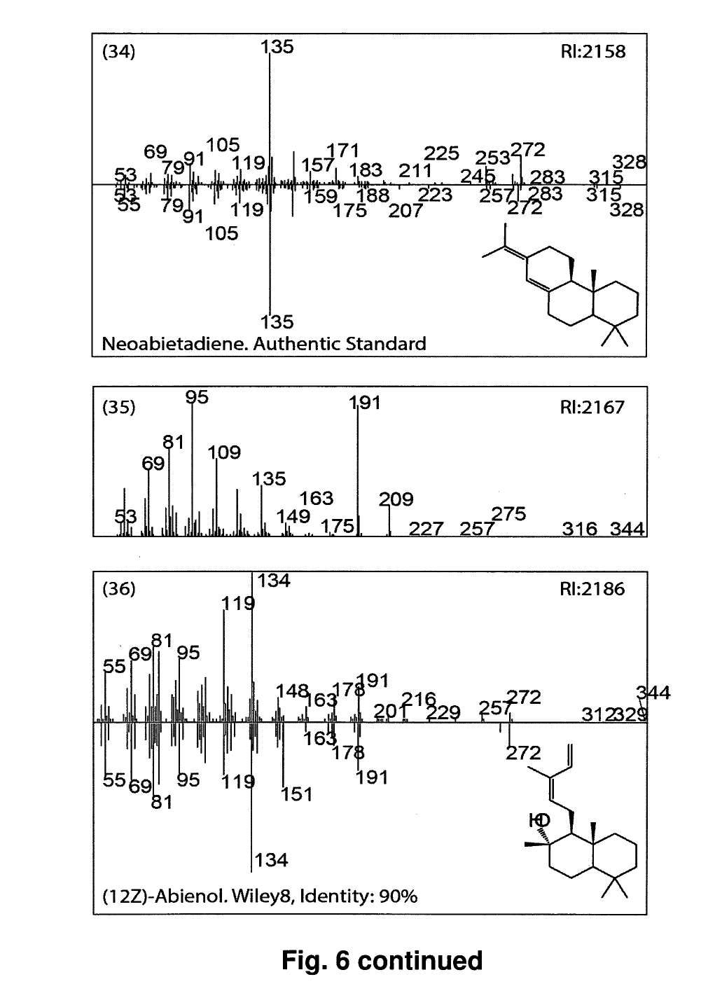

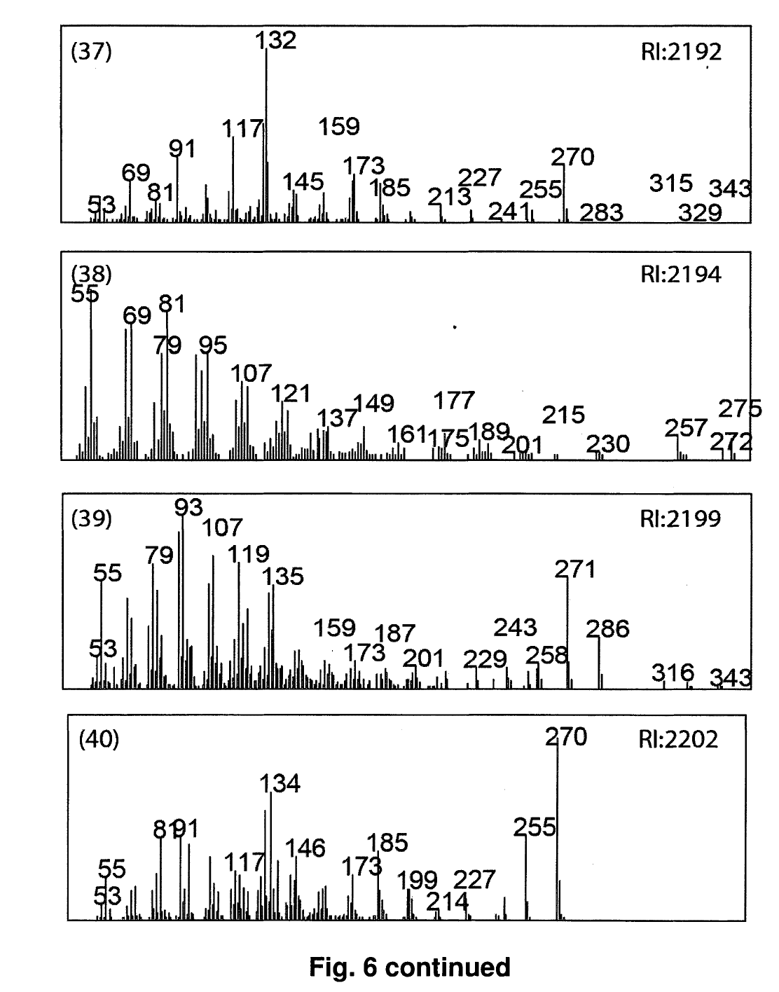

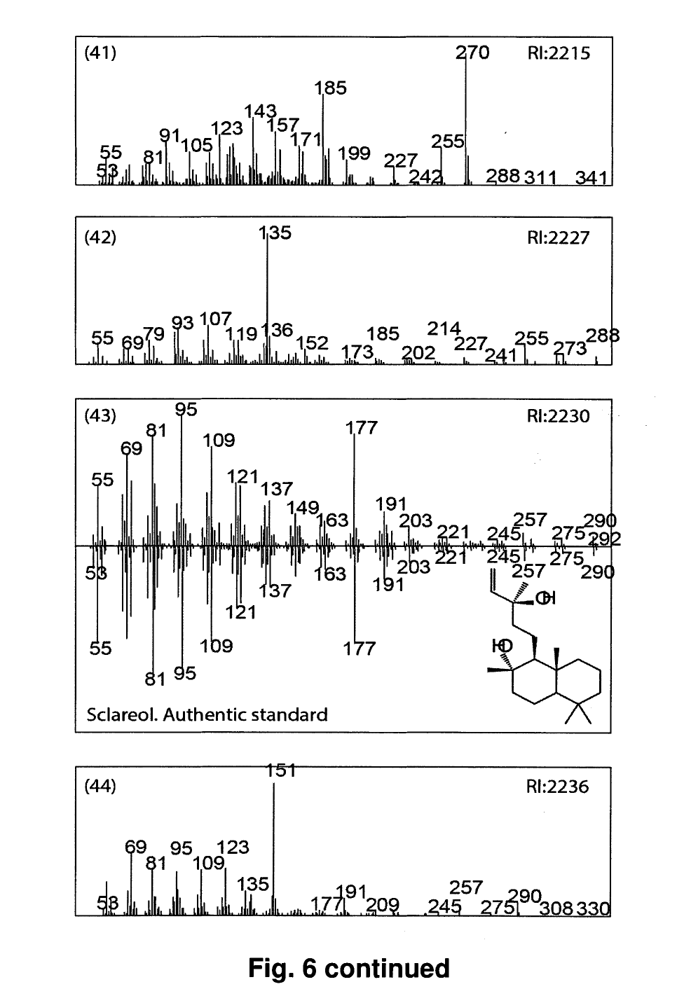

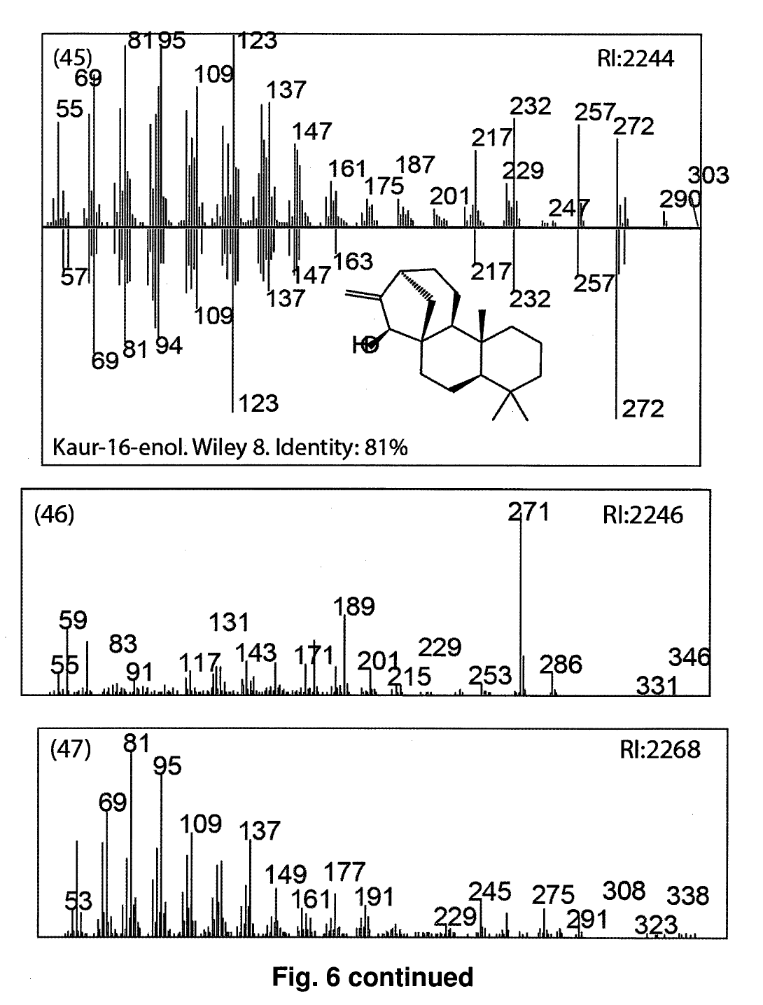

3 infiltrated leafs from each N. benthamiana line chosen and from each of these 2 leaf disc's (Ø=3 cm) were carved out and added to 1 mL n-hexane with 1 ppm 1-eicosene as internal standard (IS). The 3 replicates served as experimental replicates. Extraction was done at RT for 1 hour in an orbital shaker set at 220 rpm. Plant material was spun down and extracts were transferred to new vials. Extracts were analyzed on a Shimadzu GCMS-QP2010 Ultra using an Agilent HP-5MS column (30 m×0.250 mm i.d., 0.25 μm film thickness). Injection volume and temperature was set at 1 μL and 250° C. GC program: 50° C. for 2 min, ramp at rate 4° C. min-1 to 110° C., ramp at rate 8° C. min-1 to 250° C., ramp at rate 10° C. min-1 to 310° C. and hold for 5 min. Both He and H₂ were used as carrier gas and hence the retentions times were normalized with Kovat's retention index using 1 ppm C₇-C₃₀ Saturated Alkanes as reference. Electron impact (Ei) was used as ionization method in the mass spectrometer (MS) with the ion source temperature set to 230° C. and 70 eV. MS spectra's was recorded from 50 m/z to 350 m/z. Compound identification was done by comparison to authentic standards and comparison to reference spectra databases (Wiley Registry of Mass Spectral Data, 8th Edition, July 2006, John Wiley & Sons, ISBN: 978-0-470-04785-9). Identification was also done by C13-NMR (see below). 47 different diterpenes listed in table 1 were detected. Some of the results are also shown in FIGS. 6 and 7. Each compound was assigned a number, and the spectrum of some of the compounds is shown in FIG. 6. The compound number provided in table 1 corresponds to the compound number provided FIGS. 2 and 6. FIG. 2 shows the compound names, structures and numbers. Qualitative quantification was based on the average of the experimental replicates of the total ion chromatogram (TIC) peak area normalized to the TIC area of IS.

Semi Large Scale Production of Miltiradiene and Kovalool for NMR Analysis.

For the accumulation of 0.5-1.5 mg of diterpene for structural analysis with NMR the diTPS class II and diTPS class I combination, which yielded the compound of interest were selected (see FIG. 2B). 500 mL agrobacterium cultures containing plasmids with the p19, CfDXS, CfGGPPs, diTPS class II and diTPS class I gene respectively, were grown ON from 20 mL starter cultures. All agrobacteria lines were spun down and resuspended in H₂O with to an OD600=0.5. Whole N. benthamiana plants were submerged in the agrobacteria mix described above and infiltration was subsequently done by applying −70 kPa vacuum for 30 sec, similar to the method described in (Sainsbury, Saxena et al. 2012). After 7-8 days of growth leafs were harvested and “chopped”. Extractions were done by 0.5 L n-hexane per 100 g fresh weight leaf material. Extraction volume was reduced by rotor evaporation (Buchi, Schwitzerland) set to 35° C. and 220 mbar. Residual material was removed to a second vial whereas the n-hexane was reused for a repeated extraction. Extraction was repeated three times. Concentrated plant extract was applied on a Dual Layer Florisil/Na2SO4 6 mL PP SPE TUBE, Superleco Analytical. Elution from the column was done with a gradient eluent of n-hexane and 1-15% ethyl acetate. This was repeated 3-5 times. Fractions were analyzed with GC-MS to identify the fraction containing the diterpene of interest. Purification of miltiradiene was subsequently done on a preparative GC-MS. NMR analysis of miltiradiene was done on a Bruker 400 MHz NMR instrument.

TABLE 2A
H¹-NMR for the identification of miltiradiene

| | (Gao, Hillwig et al. 2009) | This work |
|---|---|---|
| #C | δH (ppm) | δH (ppm) |
| 7 | 1.896 (d), 1.931 (d) | 1.993 (d), 1.929 (d) |
| 8 | | |
| 9 | | |
| 10 | | |
| 11 | 2.396 (t), 2.475 (t) | 2.391 (t), 2.466 (t) |
| 12 | 5.4335 (d) | 5.42 (br. s) |
| 13 | | |
| 14 | 2.612 (2H, br. s) | 2.6 (m) |
| 15 | 2.159 (m) | 2.156 (m) |
| 16 | 0.926 (3H, d J = 2.5) | 0.98 (3H, d J = 2.5) |
| 17 | 0.999 (3H, d J = 2.5) | 1 (3H, d J = 2.5) |
| 18 | 0.8472 (3H, s) | 0.84 (3H, s) |
| 19 | 0.871 (3H, s) | 0.87 (3H, s) |
| 20 | 0.976 (3H, s) | 0.97 (3H, s) |

HPLC-HRMS-SPE-NMR Analysis of Kolavelool

The HPLC-HRMS-SPE-NMR system consisted of an Agilent 1200 chromatograph comprising quaternary pump, degasser, thermostatted column compartment, autosampler, and photodiode array detector (Santa Clara, Calif.), a Bruker micrOTOF-Q II mass spectrometer (Bruker Daltonik, Bremen, Germany) equipped with an electrospray ionization source and operated via a 1:99 flow splitter, a Knauer Smartline K120 pump for post-column dilution (Knauer, Berlin, Germany), a Spark Holland Prospekt2 SPE unit (Spark Holland, Emmen, The Netherlands), a Gilson 215 liquid handler equipped with a 1-mm needle for automated filling of 1.7-mm NMR tubes, and a Bruker Avance III 600 MHz NMR spectrometer (¹H operating frequency 600.13 MHz) equipped with a Bruker SampleJet sample changer and a cryogenically cooled gradient inverse triple-resonance 1.7-mm TCI probe-head (Bruker Biospin, Rheinstetten, Germany). Mass spectra were acquired in positive ionization mode, using drying temperature of 200° C., capillary voltage of 4100 V, nebulizer pressure of 2.0 bar, and drying gas flow of 7 L/min. A solution of sodium formate clusters was automatically injected in the beginning of each run to enable internal mass calibration. Cumulative SPE trapping of kolavelool was performed after 10 consecutive separations using a chromatographic method as follows: 0 min., 90% B; 15 min., 100% B; 20 min., 100% B; 25 min., 100% B; 26 min., 90% B with 10 min. equilibration prior to injection of 5 μL pre-fractionated sample (8.5 mg/mL in hexane). The HPLC eluate was diluted with Milli-Q water at a flow rate of 1.0 mL/min prior to trapping on 10×2 mm i.d. Resin GP (general purpose, 5-15 μm, spherical shape, polydivinyl-benzene phase) SPE cartridges from Spark Holland (Emmen, The Netherlands), and kolavelool was trapped using threshold of an extracted ion chromatogram (m/z 273.2 corresponding to [M+H−H₂O]⁺). The SPE cartridge was dried with pressurized nitrogen gas for 60 min prior to elution with chloroform-d. The HPLC was controlled by Bruker Hystar version 3.2 software, automated filling of NMR tubes were controlled by PrepGilsonST version 1.2 software, and automated NMR acquisition were controlled by Bruker IconNMR version 4.2 software. NMR data processing was performed using Bruker Topspin version 3.2 software.

NMR Analyses of Kolavelool

NMR spectra of kolavelool was recorded in chloroform-d at 300 K. ¹H and ¹³C chemical shifts were referenced to the residual solvent signal (δ 7.26 and δ 77.16, respectively). One-dimensional ¹H NMR spectrum was acquired in automation (temperature equilibration to 300 K, optimization of lock parameters, gradient shimming, and setting of receiver gain) with 30°-pulses, 3.66 s inter-pulse intervals, 64 k data points and multiplied with an exponential function corresponding to line-broadening of 0.3 Hz prior to Fourier transform. Phase-sensitive DQF-COSY and NOESY spectra were recorded using a gradient-based pulse sequence with a 20 ppm spectral width and 2 k×512 data points (processed with forward linear prediction to 1 k data points). Multiplicity-edited HSQC spectrum was acquired with the following parameters: spectral width 20 ppm for ¹H and 200 ppm for ¹³C, 2 k×256 data points (processed with forward linear prediction to 1 k data points), and 1.0 s relaxation delay. HMBC spectrum was optimized for ⁿJ_{C,H}=8 Hz and acquired using the following parameters: spectral width 20 ppm for ¹H and 240 ppm for ¹³C, 2 k×128 data points (processed with forward linear prediction to 1 k data points), and 1.0 s relaxation delay. NMR spectra of syn-isopimara-9(11), 15-diene was recorded in chloroform-d at 300 K on a Bruker Avance III 600 MHz NMR spectrometer (¹H operating frequency 600.13 MHz) equipped with a Bruker SampleCase sample changer and a cryogenically cooled gradient 5.0-mm DCH probe-head (Bruker Biospin, Rheinstetten, Germany) in a 3.0 mm o.d. NMR tube. ¹H and ¹³C chemical shifts were referenced to the residual solvent signal (δ 7.26 and δ 77.16, respectively). One-dimensional ¹H and ¹³C NMR spectrum was acquired in automation (temperature equilibration to 300 K, optimization of lock parameters, gradient shimming, and setting of receiver gain) with 30°-pulses, 3.66 s inter-pulse intervals, 64 k data points and multiplied with an exponential function corresponding to line-broadening of 0.3 and 1.0 Hz, respectively prior to Fourier transform. Phase-sensitive DQF-COSY and ROESY spectra were recorded using a gradient-based pulse sequence with a 7.4 ppm spectral width and 2 k×128 and 2 k×256 data points, respectively (processed with forward linear prediction to 1 k data points). Multiplicity-edited HSQC spectrum was acquired with the following parameters: spectral width 16 ppm for ¹H and 165 ppm for ¹³C, 2 k×256 data points (processed with forward linear prediction to 1 k data points), and 1.0 s relaxation delay. HMBC spectrum was optimized for ⁿJ_{C,H}=8 Hz and acquired using the following parameters: spectral width 7.9 ppm for ¹H and 221 ppm for ¹³C, 4 k×256 data points (processed with forward linear prediction to 1 k data points), and 1.0 s relaxation delay.

TABLE 2B
H¹- & C¹³- NMR data of (+/−)-kolavelool acquired
in chloroform-d in HPLC-HRMS-SPE-NMR mode

| | (Bomm, | (Bomm, | | |
|---|---|---|---|---|
| | Zukerman- | Zukerman- | | |
| | Schpector et al. | Schpector et al. | | |
| | 1999) | 1999) | This work | This work |
| Position | δ_{C} | δ_{H} (J in Hz) | δ_{C}^{b} | δ_{H} (J in Hz) |
| 1 | 18.2 | | 18.2 | 1.41^{a} |
| | | | | 1.53^{a} |
| 2 | 27.4 | | 27 | 2.01^{a} |
| 3 | 120.4 | 5.16 s | 120.5 | 5.17, s |
| 4 | 144.5 | | 144.6 | |
| 5 | 38.1 | | 37.4 | |
| 6 | 36.8 | | 37.1 | 1.15^{a} |
| | | | | 1.69, dt (12.0, |
| | | | | 3.0) |
| 7 | 26.8 | | 27.6 | 1.40^{a} |
| 8 | 36.1 | | 36.25 | 1.41^{a} |
| 9 | 38.3 | | 38 | |
| 10 | 46.3 | | 46.5 | 1.3^{a} |
| 11 | 31.8 | | 31.8 | 1.38^{a} |
| | | | | 1.25^{a} |
| 12 | 35.3 | | 35.4 | 1.37^{a} |
| 13 | 73.4 | | 73.2 | |
| 14 | 145.1 | 5.84 dd (17.2, | 145.2 | 5.87, dd (17.4, |
| | | 10.8) | | 10.7) |
| 15 | 111.8 | 5.07 dd (17.2, | 111.9 | 5.04, bd (10.7) |
| | | 1.5) | | 5.18, bd (17.4) |
| | | 4.99 dd (10.8, | | |
| | | 1.5) | | |
| 16 | 27.7 | 1.24 s | 27.9 | 1.25, s |
| 17 | 15.9 | 0.75 d (5.9) | 16 | 0.76, d (5.7) |
| 18 | 18 | 1.54 d (1.5) | 18 | 1.57, bs |
| 19 | 19.2 | 0.95 s | 20.11 | 0.97, s |
| 20 | 18.4 | 0.68 s | 18.5 | 0.71, s |

^{a}Coupling constants not determined due to overlap with HOD as a result of inadequate drying of cartridge in HPLC-HRMS-SPE-NMR mode; ¹H chemical shifts from HSQC experiments.
^{b13}C chemical shifts from one- and multiple-bond proton-detected 2D heteronuclear correlations.

TABLE 1

| Compound | Structure |
|---|---|
| (1) | |
| (2) | 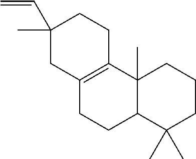 [SMILES: C=CC1(C)CCC2=C(CCC3C(C)(C)CCCC23C)C1] |
| (3) | 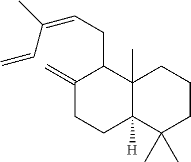 [SMILES: C=C/C(C)=C\CC1C(=C)CC[C@@H]2C(C)(C)CCCC12C] |
| (4) | |
| (5) | 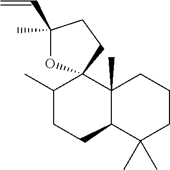 |
| (6) | 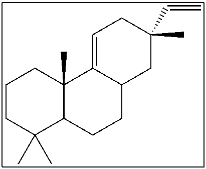 [SMILES: C=C[C@@]1(C)CC=C2C(CCC3C(C)(C)CCC[C@]23C)C1] |
| (7) | |
| (8) | |
| (9) | 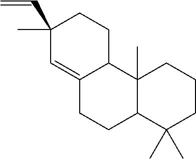 [SMILES: C=C[C@]1(C)C=C2CCC3C(C)(C)CCCC3(C)C2CC1] |
| (10) | |
| (11) | |
| (12) | |
| (13) | |
| (14) | |
| (15) | 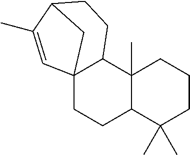 [SMILES: CC1=CC23CCC4C(C)(C)CCCC4(C)C2CCC1C3] |
| (16) | 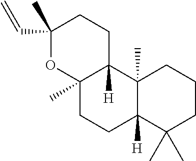 |
| (17) | 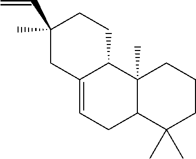 [SMILES: C=C[C@@]1(C)CC[C@H]2C(=CCC3C(C)(C)CCC[C@@]32C)C1] |
| (19) | 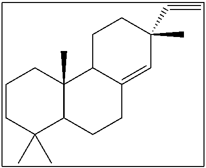 [SMILES: C=C[C@]1(C)C=C2CCC3C(C)(C)CCC[C@]3(C)C2CC1] |
| (18) | |
| (20) | 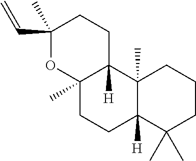 |
| (21) | 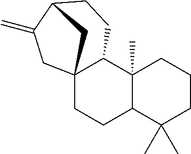 [SMILES: C=C1C[C@@]23CCC4C(C)(C)CCC[C@]4(C)[C@H]2CC[C@@H]1C3] |
| (22) | 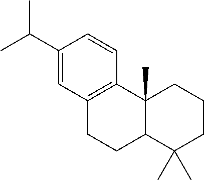 [SMILES: CC(C)c1ccc2c(c1)CCC1C(C)(C)CCC[C@@]21C] |
| (23) | 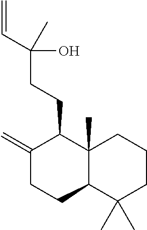 [SMILES: C=CC(C)(O)CC[C@@H]1C(=C)CC[C@@H]2C(C)(C)CCC[C@@]12C] |
| (24) | |
| (25) | 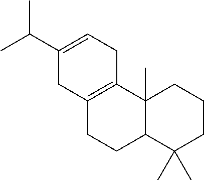 [SMILES: CC(C)C1=CCC2=C(CCC3C(C)(C)CCCC23C)C1] |
| (26) | 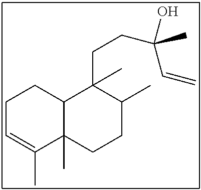 [SMILES: C=C[C@@](C)(O)CCC1(C)C(C)CCC2(C)C(C)=CCCC21] |
| (27) | 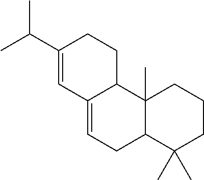 [SMILES: CC(C)C1=CC2=CCC3C(C)(C)CCCC3(C)C2CC1] |
| (28) | |
| (29) | |
| (30) | 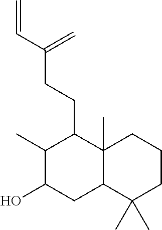 [SMILES: C=CC(=C)CCC1C(C)C(O)CC2C(C)(C)CCCC12C] |
| (31) | |
| (32) | |
| (33) | |
| (34) | 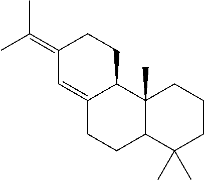 [SMILES: CC(C)=C1C=C2CCC3C(C)(C)CCC[C@@]3(C)[C@@H]2CC1] |
| (35) | |
| (36) | |
| (37) | |
| (38) | |
| (39) | |
| (40) | |
| (41) | |
| (42) | |
| (43) | 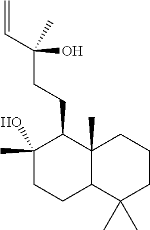 [SMILES: C=C[C@@](C)(O)CC[C@@H]1[C@@](C)(O)CCC2C(C)(C)CCC[C@]21C] |
| (44) | |
| (45) | 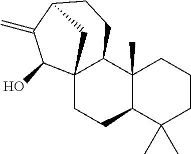 [SMILES: C=C1[C@H]2CC[C@H]3[C@]4(C)CCCC(C)(C)[C@H]4CC[C@]3(C2)[C@H]1O] |
| (46) | |
| (47) | |

### REFERENCES

Voinnet, O., S. Rivas, et al. (2003). “An enhanced transient expression system in plants based on suppression of gene silencing by the p19 protein of tomato bushy stunt virus.” The Plant Journal 33(5): 949-956.
Zerbe, P., B. Hamberger, et al. (2013). “Gene Discovery of Modular Diterpene Metabolism in Nonmodel Systems.” Plant Physiology 162(2): 1073-1091.
Sainsbury, F., P. Saxena, et al. (2012). Chapter Nine—Using a Virus-Derived System to Manipulate Plant Natural Product Biosynthetic Pathways. Methods in Enzymology. A. H. David, Academic Press. Volume 517: 185-202.

### Example 2

Production of Syn-Pimara-9,(11),15-Diene (6) for NMR Analysis.

For the structural elucidation of syn-pimara-9,(11),15-diene (6), a 0.1 L culture of a yeast strain containing OssynCPS, CfTPS3 and a GGPPs (see example 3) in a feed in time media was inoculated with a 5 mL ON culture. The culture was grown for 72 hours and harvested by adding 0.1 L of ethanol, mixing and heating to 70° C. for 20 min. After heating 0.1 L n-hexane was added, followed by horizontal shaking at 200 rpm for 1 hour. Subsequently the hexane overlay was transferred to the rotor evaporator where the volume was reduced.

Purification of Syn-Pimara-9,(11),15-Diene (6) by Solid Phase Extraction and Preparative GC-MS.

Concentrated hexane extract from yeast was applied on a Dual Layer Florisil/Na₂SO₄ 6 mL PP SPE TUBE, Superleco Analytical. Elution from the column was done with a gradient eluent of n-hexane and 1-15% ethyl acetate. This was repeated 3-5 times. Fractions were analyzed with GC-MS to identify the fraction containing the diterpene of interest, these were pooled and solvent was removed by rotor evaporation and resuspended in 1 mL n-hexane. Final purification was done on an Agilent 7890B GC installed with an Agilent 5977A inert MSD, GERSTEL Preparative Fraction Collector (PFC) AT 6890/7890 and a GERSTEL CIS 4C Bundle injection port. For separation by GC a RESTEK Rtx-5 column (30 m×0.53 mm ID×1 μm df) with H2 as carrier gas was used. At the end of this column a split piece with a split of 1:100 to the MS and the PFC, respectively. Sufficient amount of diterpene product for NMR analysis (0.5-1 mg) was obtained by 130 injection of 5 μL of extract. Injection port was put in solvent vent mode with 100 mL until 0.17 min. Injection temperature was held at 40° C. for 0.1 min followed by ramping at 12° C./sec until 320, which was held for 2 min. The GC program was set to hold at 60° C. for 1 min, ramp 30° C./min to 220° C., ramp 2° C./min to 250° C. and a final ramp of 30° C./min to 220° C., which was held for 2 min. Temperature of the transfer line from GC to PFC and the PFC itself was set to 250° C. The PFC was set to collect the peak of syn-pimara-9,(11),15-diene (6) by their retention time identified by the MS. The method for NMR analysis for structural characterization of syn-pimara-9,(11),15-diene (6) was the same as for the analysis of kovalool (see example 1)

TABLE 3
NMR data of syn-isopimara-9(11), 15-
diene^{a} acquired in chloroform-d

| | (Oikawa, Toshima | | |
|---|---|---|---|
| | et al. 2001) | This work | This work |
| position | δ_{H} (J in Hz) | δ_{C} | δ_{H} (J in Hz) |
| 1 | | 37.8 | 1.36, m |
| | | | 1.65, m |
| 2 | | 19.2 | 1.53, m |
| | | | 1.65, m |
| 3 | | 42.5 | 1.16, td (13.6, 3.9) |
| | | | 1.40, m |
| 4 | | 33.8 | |
| 5 | | 53.9 | 0.95, dd (12.3, 2.6) |
| 6 | | 22.12 | 1.46, m |
| | | | 1.66, m |
| 7 | | 36.4 | 1.01, m |
| | | | 1.89, m |
| 8 | | 31.3 | 2.28, m |
| 9 | | 149.9 | |
| 10 | | 39.4 | |
| 11 | 5.29, m | 112.6 | 5.27, ddd (6.1, 2.0, 1.5) |
| 12 | | 37.5 | 1.72, m |
| | | | 2.05, ddd (17.1, 2.8, 2.0) |
| 13 | | 34.9 | |
| 14 | | 42.8 | 1.10, dd (12.6, 10.9) |
| | | | 1.50, m |
| 15 | 5.77, dd (17.2, 11.2) | 150.5 | 5.82, dd (17.5, 10.8) |
| 16 | 4.85-4.93, m | 109.3 | 4.87, dd (10.8, 1.4) |
| | | | 4.94, dd (17.5, 1.4) |
| 17 | 0.95, s | 22.2 | 0.92, s |
| 18^{b} | 0.84, s | 33.5 | 0.85, s |
| 19^{b} | 0.84, s | 22.09 | 0.86, s |
| 20 | 0.98, s | 21.1 | 1.04, s |

^{a}Relative stereochemistry concluded on the basis of NOE correlations between H-8-H-20 and H-8-H-17 as well as the absence of correlations between H-5 and H-20.
^{b}Interchangeable

### Example 3

Construction of Yeast Strain for the Production of Diterpenes

Materials and Methods.

Table 4 summarises the coding DNA sequences (CDS) used in this study. The CDS encodes the proteins indicated in Table, but have been sequence optimized for expression in yeast.

TABLE 4
CDSs used in this study.

| CDS | Description |
|---|---|
| CfTPS1 | SEQ ID NO: 19 - endodes CfTPS1 (Coleus forskohlii |
| | diterpene synthase 2) truncated to remove putative |
| | plastid targeting sequence |
| CfTPS3 | SEQ ID NO: 20 - encodes CfTPS3 (Coleus forskohlii |
| | diterpene synthase 3) truncated to remove putative |
| | plastid targeting sequence |
| ZmAN2 | SEQ ID NO: 21 - encodes ZmAN2 (Zea Maiz diterpene |
| | synthase class II) truncated to remove putative |
| | plastid targeting sequence |
| OssynCPS | OssynCPS (Oryza sativa ditepene synthase class |
| | II) truncated to remove putative plastid |
| | targeting sequence |
| TwTPS21 | SEQ ID NO: 23 - encodes TwTPS21 |
| | (Tripterygium wilfordii diterpene synthase class |
| | II) truncated to remove putative plastid |
| | targeting sequence |
| SsSCS | SEQ ID NO: 24 - encodes SsSCS (Salvia Sclarea |
| | diterpene synthase class I) truncated to remove |
| | putative plastid targeting sequence |
| TwTPS14 | SEQ ID NO: 25 - encodes TwTPS14 |
| | (Tripterygium Wilfordii diterpene synthase class |
| | II) truncated to remove putative plastid |
| | targeting sequence |
| GGPPs | Geranylgeranyl diphosphate synthase |

TABLE 5
List of plasmids used in the study.

| pCYPCC- | pROP196 XI-5 | Rv #205 GGPPs7<−pTPI1 #219 |
|---|---|---|
| 1 | assembler 1 | |
| pCYPCC- | pROP196 XI-5 | Rv #206 GGPPs10<−pTPI1 #219 |
| 2 | assembler 1 | |
| pCYPCC- | pROP196 XI-5 | Rv #205 GGPPs7<−pPGK1 1c |
| 3 | assembler 1 | |
| pCYPCC- | pROP196 XI-5 | Rv #206 GGPPs10<−pPGK1 1c |
| 4 | assembler 1 | |
| pCYPCC- | pROP197 XI-5 | #-3 CfTPS3 <−#161pTDH3 |
| 7 | assembler 3 | |
| pCYPCC- | pVAN858 | 2c pTEF1−>#-5 CfTPS1 |
| 9 | assembler 2 | |
| pCYPCC- | pVAN858 | 2c pTEF1−>#-6 OsCPssyn |
| 10 | assembler 2 | |
| pCYPCC- | pROP197 XI-5 | #-8 SsSCS <−#161pTDH3 |
| 18 | assembler 3 | |
| pCYPCC- | pROP197 XI-5 | Res# 236 CfTPS3 co<−#161pTDH3 |
| 21 | assembler 3 | |
| pCYPCC- | pVAN858 | Res160 pTEF-2 −>CfTPS1, co |
| 42 | assembler 2 | |
| pCYPCC- | pVAN858 | Res160 pTEF-2 −>OsCPssyn |
| 44 | assembler 2 | |
| pCYPCC- | pROP197 XI-5 | SsSCS, co<−#161pTDH3 |
| 51 | assembler 3 | |

All enzymes cloned in plasmids pCYPCC7-51 were truncated to remove putative plastid targeting sequence (see sequence listing).

Abbreviation: co=codon optimized. Codon optimization for Saccharomyzes cerevisae was performed using the Geneart service from LifeTechnologies.

DNA fragments containing the enzymes of interest were USER cloned into pre-digested plasmid backbones. All plasmids constructed and used in this study are summarized in table 5. DNA fragments of interest were liberated from plasmids by Notl enzyme-digestion as linear DNA fragments suitable for yeast transformation. The plasmids are designed to accommodate integration of up to three Notl-digested fragments at the same site in the genome.

TABLE 6
Strains used and generated in this study

| Strain | CDS | Compound produced | Analysis |
|---|---|---|---|
| T2 | TwTPS14 + | Kovalool (26) | GC-MS |
| | SsSCS + GGPPs | | |
| T5 | ZmAN2 + | ent-manool (23b) | GC-MS/ |
| | SsSCS + GGPPs | | LC-MS |
| T8 | TwTPS21 + | 13S-manoyl oxide (20) | GC-MS |
| | EpTPS1 + GGPPs | | |
| EFSC4725 | CfTPS1 + | (+)-manool | GC-MS/ |
| | SsSCS + GGPPs | | LC-MS |
| EFSC4727 | OssynCPS + | syn-manool (11) | LC-MS |
| | SsSCS + GGPPs | | |
| EFSC4690 | OssynCPS + | syn-pimara-9,(11),15- | GC-MS |
| | CfTPS3 + GGPPs | diene (6), syn-isopimara- | |
| | | 7,15-diene (19) | |
| EFSC4691 | CfTPS1 + | Miltiradiene (25) | GC-MS |
| | CfTPS3 + GGPPs | | |
| EFSC4494 | CfTPS2 + | 13R-manoyl oxide | GC-MS |
| | CfTPS3 + GGPPs | | |

All strains were grown in 96 deep well plates as follows. Single colonies were inoculated in 500 μl SC-Ura in 2.2 ml 96 deep well plates and grown o/n @ 3000, 400 RPM. The following day 50 μl of the o/n culture was used as inoculum in 500 μl DELFT media with 10% sun flower oil and grown for additional 72 hours @ 30° C., 400 RPM.

Table 6 summarizes the compounds produced by the various strains. The table also indicates whether the compound was identified LC-MS and/or GC-MS. LC-MS analysis and/or GC-MS analysis were performed as described below. The numbers indicated in brackets refer to the compounds numbers shown in FIG. 2.

Extraction and LC-MS Analysis

Metabolites were extracted from the whole broth by adding 500 μl 96% Ethanol, mix and incubate @ 78° C. for 10 min. For LC-MS analysis cell debris was removed by centrifugation for 2 min at 15000 xg. Supernatant was used for LC-MS analysis. LC-MS was carried out using an Agilent 1100 Series LC (Agilent Technologies, Germany) coupled to a Bruker HCT-Ultra ion trap mass spectrometer (Bruker Daltonics, Bremen, Germany). A Zorbax SB-C18 column (Agilent; 1.8 μm, 2.1×50 mm) maintained at 35° C. was used for separation. The mobile phases were: A, water with 0.1% (v/v) HCOOH and 50 mM NaCl; B, acetonitrile with 0.1% (v/v) HCOOH. The gradient program was: 0 to 1 min, isocratic 50% B; 1 to 10 min, linear gradient 50 to 95% B; 10 to 11.4 min, isocratic 98% B; 11.4 to 17 min, isocratic 50% B. The flow rate was 0.2 mL min-1. The mass spectrometer was run in alternating positive/negative mode and the range m/z 100-800 was acquired.

Extraction GC-MS Analysis

Metabolites were extracted from the whole broth by adding 500 μl 96% Ethanol, mix and incubate @ 78° C. for 10 min. Solvent and liquids were removed by freeze drying. 500 μL of hexane including 1 mg/L 1-eicosene as internal standard (ISTD), was used for extraction at room temperature for ½ an hour. Particles in the extraction media was removed by centrifugation for 2 min at 15000 xg. After extraction, the solvent was transferred into new 1.5-mL glass vials and stored at −20° C. until GC-MS analysis. One microliter of hexane extract was injected into a Shimadzu GC-MS-QP2010 Ultra. Separation was carried out using an Agilent HP-5MS column (20 m 0.180 mm i.d., 0.18 μm film thickness) with purge flow of 4 mL min⁻¹ for 1 min, using H₂ as carrier gas. The GC temperature program was 60° C. for 1 min, ramp at rate 30° C. min⁻¹ to 180° C., ramp at rate 10° C. min⁻¹ to 250° C., ramp at rate 30° C. min⁻¹ to 320° C., and hold for 3 min. Injection temperature was set at 250° C. in splitless mode. Column flow and pressure was set to 5. mL min⁻¹ and 66.7 kPa yielding a linear velocity of 66.5 cm s⁻¹. Ion source and transfer line for mass spectrometer (MS) was set to 300° C. and 280° C. respectively. MS was set in scan mode from m/z 50 to m/z 350 with a scan width of 0.5 s. Solvent cutoff was 4 min.

1. A method of producing a terpene, comprising:
(a) providing a host organism comprising
i. A heterologous nucleic acid encoding a diTPS of class II,
ii. A heterologous nucleic acid encoding a diTPS of class I,
with the proviso that the diTPS of class II and the diTPS of class I are not from same species; and with the proviso that when the diTPS of class II is SsLPPS then the diTPS of class I is not CfTPS3, CfTPS4 or EpTPS8 and when the diTPS of class I is EpTPS8, then the diTPS of class II is not CfTPS2 or SsLPPS;
(b) incubating the host organism in the presence of geranylgeranyl pyrophosphate (GGPP) under conditions allowing growth of the host organism; and
c) Optionally isolating diterpene from the host organism.

2. The method of claim 1, wherein the diterpene is a C₂₀-molecule containing a decalin core and up to 3 oxygen molecules.

3. The method of claim 1, wherein the diterpene is a C₂₀-molecule containing a core structure of formula I, II, III, IV, V, VI, IX or X:

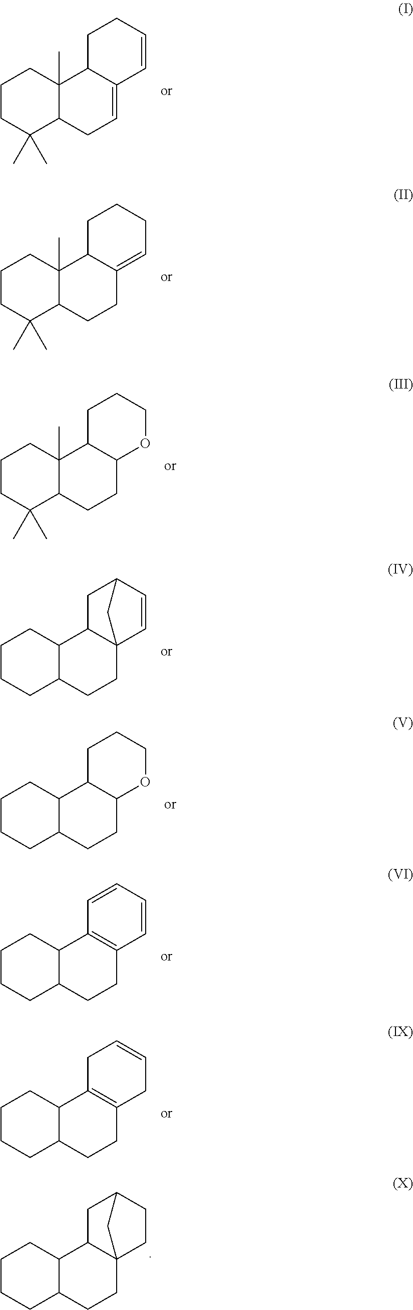

4. The method of claim 3, wherein the diterpene is a C₂₀-molecule containing a cores structure of formula I, II, III, IV, V, VI, IX or X substituted at one or more positions by one or more groups comprising:
(a) alkyl, wherein the alkyl is linear or branched;
(b) alkenyl; and
(c) hydroxyl.

5. The method of claim 1, wherein the diterpene is a C₂₀-molecule containing a decalin substituted at the 10 position with C₅-alkenyl chain, a hydroxyl, a methyl group and/or ═C.

6. The method of claim 1, wherein the diterpene is a C₂₀-molecule consisting of 20 carbon atoms, with up to three oxygen atoms and hydrogen atoms, wherein the molecule contains a core structure of formula I, II, III, IV, VI, X, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL and/or XLI.

7. The method of claim 1, wherein the diterpene is a product of any one of reactions VII to XIX.

8. The method of claim 1, wherein the diterpene is any one of compounds 1 to 47 of Table 1.

9. A host organism, comprising:
i. A heterologous nucleic acid encoding a diTPS of class II,
ii. A heterologous nucleic acid encoding a diTPS of class I,
with the proviso that the diTPS of class II and the diTPS of class I is not from the same species.

10. The method claim 1, wherein the diTPS of class II:
(a) is a polypeptide sharing at least 30% sequence identity with the amino acid sequence of SEQ ID NO:6 or AtCPS having an amino acid sequence as shown in FIG. 5;
(b) contains D/E-X-D-D motif, wherein X is a naturally occurring amino acid;
(c) is syn-CPP type diTPS, ent-CPP type diTPS, (+)-CPP type diTPS, LPP type diTPS or LPP type diTPS;
(d) is a polypeptide having at least 70% identity to the amino acid sequence set forth in SEQ ID NO:1, SEQ ID NO:2, SEQ ID NO:3, SEQ ID NO:4, SEQ ID NO:5, SEQ ID NO:7, SEQ ID NO:8, SEQ ID NO:10, SEQ ID NO:14, SEQ ID NO:15 or SEQ ID NO:16;
(e) is an enzyme capable of catalysing reactions I to V;
(f) is a polypeptide having at least 70% identity to the amino acid sequence set forth in SEQ ID NO:6, with the proviso that the diTPS of class I is not ScSCS, CfTPS3, CfTPS4 or EpTPS8;
(q) is a polypeptide having at least 70% identity to the amino acid sequence set forth in SEQ ID NO:17, with the proviso that the diTPS of class I is not CfTPS3, CfTPS4 or EpTPS8;
(h) is an enzyme capable of catalysing at least one of the reactions XXXIII, XXXIV, XXXV, XXXVI; or
(i) is a polypeptide having at least 70% identity to the amino acid sequence set for in SEQ ID NO:28, with the proviso that the diTPS of class I is not MvTPS5.

11-12. (canceled)

13. The method of claim 1, wherein the diTPS of class I:
(a) is a polypeptide having at least 30% sequence identity with the amino acid sequence of SEQ ID NO:11 or AtEKS having an amino acid sequence as shown in FIG. 4;
(b) contains D-D-X—X-D/E motif, wherein X is a naturally occurring amino acid;
(c) is EpTPS8, EpTPS23, SsSCS, CfTPS3, CfTPS4, MvTPS5, TwTPS2, EpTPS1 or CfTPS14;
(d) is a polypeptide having at least 70% identity to the amino acid sequence set forth in SEQ ID NO:10;
(e) is an enzyme capable of catalysing any one of the reactions VII to XIX;
(f) is an enzyme capable of catalysing at least one of the reactions X, XXII, XXIV, XXX, XXXI and XXXII;
(g) is a polypeptide having at least 70% identity to the amino acid sequence set forth in SEQ ID NO:11, with the proviso that the diTPS of class II is not SsLPPS;
(h) is a polypeptide having at least 70% identity to the amino acid sequence set forth in SEQ ID NO:12, with the proviso that the diTPS of class II is not CfTPS2;
(i) is a polypeptide having at least 70% identity to the amino acid sequence set forth in SEQ ID NO:18, with the proviso that the diTPS of class II is not MvTPS1;
(j) is a polypeptide having at least 70% identity to the amino acid sequence set forth in SEQ ID NO:12, with the proviso that the diTPS of class II is not CfTPS2 or SsLPPS;
(k) is a polypeptide having at least 70% identity to the amino acid sequence set forth in SEQ ID NO:9, with the proviso that the diTPS of class II is not CfTPS2 or SsLPPS; or
(l) is a polypeptide having at least 70% identity to the amino acid sequence set forth in SEQ ID NO:13, with the proviso that the diTPS of class II is not CfTPS2 or SsLPPS.

14-15. (canceled)

16. A polypeptide having at least 70% identity to the amino acid sequence set forth in SEQ ID NO:2, SEQ ID NO:4, SEQ ID NO:5, SEQ ID NO:7, SEQ ID NO:8, SEQ ID NO:10, SEQ ID NO:14, SEQ ID NO:15 or SEQ ID NO:16.

17-41. (canceled)

42. The method of claim 1, wherein the host organism further comprises one or more heterologous nucleic acids encoding enzymes involved in the biosynthesis of GGPP.

43. The method of claim 1, wherein the enzymes have at least 70% identity to the amino acid sequence set forth in SEQ ID NO: 26 or SEQ ID NO:27.

44. The method of claim 1, wherein the host organism is a microorganism or a plant.

45. The method of claim 44, wherein the microorganism is yeast.

46. (canceled)

47. A method of producing a diterpene, comprising:
(a) providing a host organism of claim 9;
(b) preparing an extract of the host organism;
(c) providing GGPP; and
(d) incubating the extract with GGPP,
thereby producing a diterpene.

48. A method for producing kolavelool, comprising:
(a) providing a host organism comprising:
i. a heterologous nucleic acid encoding a diTPS of class II,
ii. a heterologous nucleic acid encoding a diTPS of class I,
(b) incubating the host organism in the presence of geranylgeranyl pyrophosphate (GGPP) under conditions allowing growth of the host organism; and
(c) isolating kolavelool from the host organism.

49. The method of claim 48, wherein the diTPS of class II:
(a) is capable of catalysing reaction XXXV; or
(b) has at least 70% identity to the amino acid sequence set for in SEQ ID NO:8.

50. (canceled)

51. The method of claim 48, wherein the diTPS of class I:
(a) is capable of catalysing reaction XXXVII; or
(b) has at least 70% identity to the amino acid sequence set forth in SEQ ID NO:11.

52. (canceled)

53. The host organism of claim 9, wherein the diTPS of class II:
(a) is a polypeptide sharing at least 30% sequence identity with the amino acid sequence of SEQ ID NO:6 or AtCPS having an amino acid sequence as shown in FIG. 5;
(b) contains D/E-X-D-D motif, wherein X is a naturally occurring amino acid;
(c) is syn-CPP type diTPS, ent-CPP type diTPS, (+)-CPP type diTPS, LPP type diTPS or LPP type diTPS;
(d) is a polypeptide having at least 70% identity to the amino acid sequence set forth in SEQ ID NO:1, SEQ ID NO:2, SEQ ID NO:1, SEQ ID NO:4, SEQ ID NO:5, SEQ ID NO:7, SEQ ID NO:8, SEQ ID NO:10, SEQ ID NO:14, SEQ ID NO:15 or SEQ ID NO:16;
(e) is an enzyme capable of catalysing reactions I to V;
(f) is a polypeptide having at least 70% identity to the amino acid sequence set forth in SEQ ID NO:6, with the proviso that the diTPS of class I is not ScSCS, CfTPS3, CfTPS4 or EpTPS8;
(g) is a polypeptide having at least 70% identity to the amino acid sequence set forth in SEQ ID NO:17, with the proviso that the diTPS of class I is not CfTPS3, CfTPS4 or EpTPS8;
(h) is an enzyme capable of catalysing at least one of the reactions XXXIII, XXXIV, XXXV, XXXVI; or
(i) is a polypeptide having at least 70% identity to the amino acid sequence set for in SEQ ID NO:28, with the proviso that the diTPS of class I is not MvTPS5.

54. The host organism of claim 9, wherein the diTPS of class I:
(a) is a polypeptide having at least 30% sequence identity with the amino acid sequence of SEQ ID NO:11 or AtEKS having an amino acid sequence as shown in FIG. 4;
(b) contains D-D-X—X-D/E motif, wherein X is a naturally occurring amino acid;
(c) is EpTPS8, EpTPS23, SsSCS, CfTPS3, CfTPS4, MvTPS5, TwTPS2, EpTPS1 or CfTPS14;
(d) is a polypeptide having at least 70% identity to the amino acid sequence set forth in SEQ ID NO:10;
(e) is an enzyme capable of catalysing any one of the reactions VII to XIX;
(f) is an enzyme capable of catalysing at least one of the reactions X, XXII, XXIV, XXX, XXXI and XXXII;
(g) is a polypeptide having at least 70% identity to the amino acid sequence set forth in SEQ ID NO:11, with the proviso that the diTPS of class II is not SsLPPS;
(h) is a polypeptide having at least 70% identity to the amino acid sequence set forth in SEQ ID NO:12, with the proviso that the diTPS of class II is not CfTPS2;
(i) is a polypeptide having at least 70% identity to the amino acid sequence set forth in SEQ ID NO:18, with the proviso that the diTPS of class II is not MvTPS1;
(j) is a polypeptide having at least 70% identity to the amino acid sequence set forth in SEQ ID NO:12, with the proviso that the diTPS of class II is not CfTPS2 or SsLPPS;
(k) is a polypeptide having at least 70% identity to the amino acid sequence set forth in SEQ ID NO:9, with the proviso that the diTPS of class II is not CfTPS2 or SsLPPS; or
(l) is a polypeptide having at least 70% identity to the amino acid sequence set forth in SEQ ID NO:13, with the proviso that the diTPS of class II is not CfTPS2 or SsLPPS.

55. The host organism of claim 9, wherein the host organism further comprises one or more heterologous nucleic acids encoding enzymes involved in the biosynthesis of GGPP.

56. The host organism of claim 9, wherein the enzymes comprises at least 70% identity to the amino acid sequence set forth in SEQ ID NO:26, SEQ ID NO:27.

57. The host organism of claim 9, wherein the host organism is a microorganism.

58. The host organism of claim 57, wherein the microorganism is yeast.
